SEQUENCE LISTING (188 sequences)

SEQ ID NO: 1 - protein, 8 aa, Unknown
  CD160 Binding Domain HCDR1
GFIFSNYA

SEQ ID NO: 2 - protein, 8 aa, Unknown
  CD160 Binding Domain HCDR2
VNSGGANT

SEQ ID NO: 3 - protein, 12 aa, Unknown
  CD160 Binding Domain HCDR3
ATHNYESTVFDY

SEQ ID NO: 4 - protein, 5 aa, Unknown
  CD160 Binding Domain LCDR1
SSVSY

SEQ ID NO: 5 - protein, 3 aa, Unknown
  CD160 Binding Domain LCDR2
ETS

SEQ ID NO: 6 - protein, 10 aa, Unknown
  CD160 Binding Domain LCDR3
QQWNYPSMYT

SEQ ID NO: 7 - protein, 8 aa, Unknown
  CD160 Binding Domain HCDR1
GFTFSNYG

SEQ ID NO: 8 - protein, 8 aa, Unknown
  CD160 Binding Domain HCDR2
VSNGGGNT

SEQ ID NO: 9 - protein, 12 aa, Unknown
  CD160 Binding Domain HCDR3
VTHNYESTVFDY

SEQ ID NO: 10 - protein, 8 aa, Unknown
  CD160 Binding Domain HCDR1
GFSLTSYG

SEQ ID NO: 11 - protein, 7 aa, Unknown
  CD160 Binding Domain HCDR2
IWNDGGT

SEQ ID NO: 12 - protein, 6 aa, Unknown
  CD160 Binding Domain HCDR3
ARGWTH

SEQ ID NO: 13 - protein, 6 aa, Unknown
  CD160 Binding Domain LCDR1
EDIYNN

SEQ ID NO: 14 - protein, 3 aa, Unknown
  CD160 Binding Domain LCDR2
YAS

SEQ ID NO: 15 - protein, 9 aa, Unknown
  CD160 Binding Domain LCDR3
LQDSEYPPT

SEQ ID NO: 16 - protein, 8 aa, Unknown
  CD160 Binding Domain HCDR2
ISTGDDNT

SEQ ID NO: 17 - protein, 12 aa, Unknown
  CD160 Binding Domain HCDR3
ATHNYESTIFDY

SEQ ID NO: 18 - protein, 8 aa, Unknown
  CD160 Binding Domain HCDR1
GFSLTNYG

SEQ ID NO: 19 - protein, 6 aa, Unknown
  CD160  Binding Domain HCDR3
ARGMEA

SEQ ID NO: 20 - protein, 6 aa, Unknown
  CD160 Binding Domain LCDR1
QNINNY

SEQ ID NO: 21 - protein, 3 aa, Unknown
  CD160 Binding Domain LCDR2
NTN

SEQ ID NO: 22 - protein, 8 aa, Unknown
  CD160 Binding Domain LCDR3
LQHNSGYT

SEQ ID NO: 23 - protein, 8 aa, Unknown
  CD160 binding domain HCDR1
GFTFSNYA

SEQ ID NO: 24 - protein, 8 aa, Unknown
  CD160 binding domain HCDR2
INSGGGDS

SEQ ID NO: 25 - protein, 12 aa, Unknown
  CD160 binding domain HCDR3
ATHNYGSTIFDY

SEQ ID NO: 26 - protein, 8 aa, Unknown
  CD160 binding domain HCDR1
GFSLTSHG

SEQ ID NO: 27 - protein, 6 aa, Unknown
  CD160 binding domain HCDR3
ARGLAY

SEQ ID NO: 28 - protein, 6 aa, Unknown
  CD160 binding domain LCDR1
EDIYSD

SEQ ID NO: 29 - protein, 3 aa, Unknown
  CD160 binding domain LCDR2
NAN

SEQ ID NO: 30 - protein, 9 aa, Unknown
  CD160 binding domain LCDR3
QQYNNYPWT

SEQ ID NO: 31 - protein, 6 aa, Unknown
  CD160 binding domain HCDR3
ARGMDA

SEQ ID NO: 32 - protein, 3 aa, Unknown
  CD160 binding domain LCDR2
YAN

SEQ ID NO: 33 - protein, 9 aa, Unknown
  CD160 binding domain LCDR3
QQYNNYPFT

SEQ ID NO: 34 - protein, 8 aa, Unknown
  CD160 binding domain HCDR2
ISSGGGDT

SEQ ID NO: 35 - protein, 12 aa, Unknown
  CD160 binding domain HCDR3
ATHNYESTVLIT

SEQ ID NO: 36 - protein, 6 aa, Unknown
  CD160 binding domain HCDR3
ARGFEY

SEQ ID NO: 37 - protein, 6 aa, Unknown
  CD160 binding domain LCDR1
QNVGKN

SEQ ID NO: 38 - protein, 9 aa, Unknown
  CD160 binding domain LCDR3
LQYNYNPWT

SEQ ID NO: 39 - protein, 8 aa, Unknown
  CD160 binding domain HCDR2
INTSGGST

SEQ ID NO: 40 - protein, 18 aa, Unknown
  CD160 binding domain HCDR3
ATIYYHDGSYYYAGWFAY

SEQ ID NO: 41 - protein, 6 aa, Unknown
  CD160 binding domain LCDR1
ESVSTL

SEQ ID NO: 42 - protein, 3 aa, Unknown
  CD160 binding domain LCDR2
GAS

SEQ ID NO: 43 - protein, 9 aa, Unknown
  CD160 binding domain LCDR3
QQSWNGPWT

SEQ ID NO: 44 - protein, 8 aa, Unknown
  CD160 binding domain HCDR2
ISSGRGNT

SEQ ID NO: 45 - protein, 12 aa, Unknown
  CD160 binding domain HCDR3
VTHNYESTIFDY

SEQ ID NO: 46 - protein, 8 aa, Unknown
  CD160 binding domain HCDR1
GFSLISYG

SEQ ID NO: 47 - protein, 8 aa, Unknown
  CD160 binding domain HCDR1
RFTFSNYA

SEQ ID NO: 48 - protein, 6 aa, Unknown
  CD160 binding domain HCDR3
ARGFEH

SEQ ID NO: 49 - protein, 6 aa, Unknown
  CD160 binding domain LCDR1
EDISND

SEQ ID NO: 50 - protein, 3 aa, Unknown
  CD160 binding domain LCDR2
AAS

SEQ ID NO: 51 - protein, 9 aa, Unknown
  CD160 binding domain LCDR3
QQSYKYPLT

SEQ ID NO: 52 - protein, 8 aa, Unknown
  CD160 binding domain HCDR2
ISNGGGNT

SEQ ID NO: 53 - protein, 12 aa, Unknown
  CD160 binding domain HCDR3
VAHNYESTVFDY

SEQ ID NO: 54 - protein, 8 aa, Unknown
  CD160 binding domain HCDR2
IITGVGNT

SEQ ID NO: 55 - protein, 12 aa, Unknown
  CD160 binding domain HCDR3
EHITTSPPFLIT

SEQ ID NO: 56 - protein, 112 aa, Unknown
  VH
QVNLQESGPGLVQPSETLSLTCTVSGFSLTSYGVYWVRQPPGKGLEWMGRIWNDGGTDYNPALKSRLSISRDTSKSQVFLKLNSLQTEDTAIYFCARGWTHWGQGTLVTVSS

SEQ ID NO: 57 - protein, 107 aa, Unknown
  VL
DIQMTQSPASLSASLGETVSIECLASEDIYNNLAWYQQKPGKSPQLLIYYASSLQDGVPSRFSGSGSGTQYSLKINSLESEDVATYFCLQDSEYPPTFGGGTKLELK

SEQ ID NO: 58 - protein, 119 aa, Unknown
  VH
EVKLVESGGGLVQPGRPLKLSCAASGFTFSNYGMAWVRQAPKKGLEWVATISTGDDNTYYRDSVKGRFTISRDNAKNTQYLQMDSLRSEDTATYYCATHNYESTIFDYWGQGVMVTVSS

SEQ ID NO: 59 - protein, 107 aa, Unknown
  VL
DIAITQSPASMTASPGERVTITCRASSSVSYMHWYQQKPGASPKPWIYETSKLASGVPDRFSGSGSGTSYSLTINNMEAEDAATYYCQQWNYPSMYTFGAGTKLELK

SEQ ID NO: 60 - protein, 119 aa, Unknown
  VH
EVQLVESGGGFVQPGGSLKLSCAASGFTFSNYGMAWVRQAPKKGLEWVASISSGRGNTYYRDSVKGRFTISRDNAKNTQYLQMDSLRSEDTATYYCVTHNYESTIFDYWGQGVMVTVSS

SEQ ID NO: 61 - protein, 107 aa, Unknown
  VL
EIVLTQSPTTMTASPGEKVTIICRASSSVSYMHWYQQKPGASPKPWIYETSKLASGVPDRFSGSGSGTSYSLTFNNMEAEDAATYYCQQWNYPSMYTFGAGTKLELK

SEQ ID NO: 62 - protein, 235 aa, Unknown
  scFv sequence
QVNLQESGPGLVQPSETLSLTCTVSGFSLTSYGVYWVRQPPGKGLEWMGRIWNDGGTDYNPALKSRLSISRDTSKSQVFLKLNSLQTEDTAIYFCARGWTHWGQGTLVTVSSGGGGSGGGGSGGGGSDIQMTQSPASLSASLGETVSIECLASEDIYNNLAWYQQKPGKSPQLLIYYASSLQDGVPSRFSGSGSGTQYSLKINSLESEDVATYFCLQDSEYPPTFGGGTKLELKR

SEQ ID NO: 63 - protein, 242 aa, Unknown
  scFv sequence
EVKLVESGGGLVQPGRPLKLSCAASGFTFSNYGMAWVRQAPKKGLEWVATISTGDDNTYYRDSVKGRFTISRDNAKNTQYLQMDSLRSEDTATYYCATHNYESTIFDYWGQGVMVTVSSGGGGSGGGGSGGGGSDIAITQSPASMTASPGERVTITCRASSSVSYMHWYQQKPGASPKPWIYETSKLASGVPDRFSGSGSGTSYSLTINNMEAEDAATYYCQQWNYPSMYTFGAGTKLELKR

SEQ ID NO: 64 - protein, 242 aa, Unknown
  scFv sequence
EVQLVESGGGFVQPGGSLKLSCAASGFTFSNYGMAWVRQAPKKGLEWVASISSGRGNTYYRDSVKGRFTISRDNAKNTQYLQMDSLRSEDTATYYCVTHNYESTIFDYWGQGVMVTVSSGGGGSGGGGSGGGGSEIVLTQSPTTMTASPGEKVTIICRASSSVSYMHWYQQKPGASPKPWIYETSKLASGVPDRFSGSGSGTSYSLTFNNMEAEDAATYYCQQWNYPSMYTFGAGTKLELKR

SEQ ID NO: 65 - protein, 21 aa, Unknown
  CD8a transmembrane domain
IYIWAPLAGTCGVLLLSLVIT

SEQ ID NO: 66 - protein, 27 aa, Unknown
  CD28 transmembrane domain
FWVLVVVGGVLACYSLLVTVAFIIFWV

SEQ ID NO: 67 - protein, 39 aa, Unknown
  CD28 endodomain
RSKRSRLLHSDYMNMTPRRPGPTRKHYQPYAPPRDFAAY

SEQ ID NO: 68 - protein, 37 aa, Unknown
  OX40 endodomain
RRDQRLPPDAHKPPGGGSFRTPIQEEQADAHSTLAKI

SEQ ID NO: 69 - protein, 42 aa, Unknown
  41-BB endodomain
KRGRKKLLYIFKQPFMRPVQTTQEEDGCSCRFPEEEEGGCEL

SEQ ID NO: 70 - protein, 114 aa, Unknown
  CD3 zeta endodomain
RSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 153 aa, Unknown
  CD28Z
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 189 aa, Unknown
  CD28OXZ
RSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 20 aa, Unknown
  signal peptide
METDTLLLWVLLLWVPGSTG

SEQ ID NO: 74 - protein, 234 aa, Unknown
  hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 75 - protein, 46 aa, Unknown
  human CD8 stalk
TTTPAPRPPTPAPTIASQPLSLRPEACRPAAGGAVHTRGLDFACDI

SEQ ID NO: 76 - protein, 20 aa, Unknown
  human IgG1 hinge
AEPKSPDKTHTCPPCPKDPK

SEQ ID NO: 77 - protein, 237 aa, Unknown
  IgG1 Hinge-Fc
AEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 78 - protein, 236 aa, Unknown
  IgG1 Hinge - Fc modified to remove Fc receptor recognition motifs
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 79 - protein, 520 aa, Unknown
  CAR sequence
METDTLLLWVLLVWIPGSTGQVNLQESGPGLVQPSETLSLTCTVSGFSLTSYGVYWVRQPPGKGLEWMGRIWNDGGTDYNPALKSRLSISRDTSKSQVFLKLNSLQTEDTAIYFCARGWTHWGQGTLVTVSSGGGGSGGGGSGGGGSDIQMTQSPASLSASLGETVSIECLASEDIYNNLAWYQQKPGKSPQLLIYYASSLQDGVPSRFSGSGSGTQYSLKINSLESEDVATYFCLQDSEYPPTFGGGTKLELKRSDPTTTPAPRPPTPAPTIASQPLSLRPEACRPAAGGAVHTRGLDFACDIFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 488 aa, Unknown
  CAR sequence
METDTLLLWVLLLWVPGSTGEVKLVESGGGLVQPGRPLKLSCAASGFTFSNYGMAWVRQAPKKGLEWVATISTGDDNTYYRDSVKGRFTISRDNAKNTQYLQMDSLRSEDTATYYCATHNYESTIFDYWGQGVMVTVSSGGGGSGGGGSGGGGSDIAITQSPASMTASPGERVTITCRASSSVSYMHWYQQKPGASPKPWIYETSKLASGVPDRFSGSGSGTSYSLTINNMEAEDAATYYCQQWNYPSMYTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 488 aa, Unknown
  CAR sequence
METDTLLLWVLLLWVPGSTGEVQLVESGGGFVQPGGSLKLSCAASGFTFSNYGMAWVRQAPKKGLEWVASISSGRGNTYYRDSVKGRFTISRDNAKNTQYLQMDSLRSEDTATYYCVTHNYESTIFDYWGQGVMVTVSSGGGGSGGGGSGGGGSEIVLTQSPTTMTASPGEKVTIICRASSSVSYMHWYQQKPGASPKPWIYETSKLASGVPDRFSGSGSGTSYSLTFNNMEAEDAATYYCQQWNYPSMYTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 402 aa, Unknown
  iCasp9 sequence
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 83 - protein, 157 aa, Unknown
  RQR8 sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 84 - protein, 13 aa, Unknown
  Heavy chain CDR1
KASGYTFTRYTMH

SEQ ID NO: 85 - protein, 16 aa, Unknown
  Heavy chain CDR2
INPSRGYTNYNQKFKD

SEQ ID NO: 86 - protein, 10 aa, Unknown
  Heavy chain CDR3
YYDDHYCLDY

SEQ ID NO: 87 - protein, 10 aa, Unknown
  Light chain CDR1
SASSSVSYMN

SEQ ID NO: 88 - protein, 11 aa, Unknown
  Light chain CDR2
RWIYDTSKLAS

SEQ ID NO: 89 - protein, 9 aa, Unknown
  Light chain CDR3
QQWSSNPFT

SEQ ID NO: 90 - protein, 242 aa, Unknown
  scFv sequence
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 91 - protein, 8 aa, Unknown
  Heavy chain CDR1
GYSFTGYT

SEQ ID NO: 92 - protein, 8 aa, Unknown
  Heavy chain CDR2
INPYKGVS

SEQ ID NO: 93 - protein, 15 aa, Unknown
  Heavy chain CDR3
ARSGYYGDSDWYFDV

SEQ ID NO: 94 - protein, 6 aa, Unknown
  Light chain CDR1
QDIRNY

SEQ ID NO: 95 - protein, 3 aa, Unknown
  Light chain CDR2
YTS

SEQ ID NO: 96 - protein, 9 aa, Unknown
  Light chain CDR3
QQGNTLPWT

SEQ ID NO: 97 - protein, 245 aa, Unknown
  scFv sequence
DIQMTQSPSSLSASVGNRVTITCRASQDIRNYLNWYQQKPGKAPKLLIYYTSRLESGVPSRFSGSGSGTDYTLTISSLQPEDFATYYCQQGNTLPWTFGQGTKVEIKSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 98 - protein, 8 aa, Unknown
  Heavy chain CDR1
GFTFSSFP

SEQ ID NO: 99 - protein, 8 aa, Unknown
  Heavy chain CDR2
ISTSGGRT

SEQ ID NO: 100 - protein, 12 aa, Unknown
  Heavy chain CDR3
AKFRQYSGGFDY

SEQ ID NO: 101 - protein, 8 aa, Unknown
  Light chain CDR1
SGNIENNY

SEQ ID NO: 102 - protein, 3 aa, Unknown
  Light chain CDR2
DDD

SEQ ID NO: 103 - protein, 9 aa, Unknown
  Light chain CDR3
HSYVSSFNV

SEQ ID NO: 104 - protein, 245 aa, Unknown
  scFv sequence
DFMLTQPHSVSESPGKTVIISCTLSSGNIENNYVHWYQQRPGRAPTTVIFDDDKRPDGVPDRFSGSIDRSSNSASLTISGLQTEDEADYYCHSYVSSFNVFGGGTKLTVLSGGGGSGGGGSGGGGSEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSS

SEQ ID NO: 105 - protein, 20 aa, Unknown
  Signal peptide
METDTLLLWVLLLWVPGSTG

SEQ ID NO: 106 - protein, 21 aa, Unknown
  Signal peptide
MGTSLLCWMALCLLGADHADG

SEQ ID NO: 107 - protein, 26 aa, Unknown
  IgG1 hinge
AEPKSPDKTHTCPPCPKDPKSGGGGS

SEQ ID NO: 108 - protein, 45 aa, Unknown
  CD8 stalk
TTTPAPRPPTPAPTIASQPLSLRPEACRPAAGGAVHTRGLDFACD

SEQ ID NO: 109 - protein, 7 aa, Unknown
  Spacer
SGGGSDP

SEQ ID NO: 110 - protein, 554 aa, Unknown
  bi-specific molecule
METDTLLLWVLLVWIPGSTGQVNLQESGPGLVQPSETLSLTCTVSGFSLTSYGVYWVRQPPGKGLEWMGRIWNDGGTDYNPALKSRLSISRDTSKSQVFLKLNSLQTEDTAIYFCARGWTHWGQGTLVTVSSGGGGSGGGGSGGGGSDIQMTQSPASLSASLGETVSIECLASEDIYNNLAWYQQKPGKSPQLLIYYASSLQDGVPSRFSGSGSGTQYSLKINSLESEDVATYFCLQDSEYPPTFGGGTKLELKRSDPTTTPAPRPPTPAPTIASQPLSLRPEACRPAAGGAVHTRGLDFACDSGGGGSDFMLTQPHSVSESPGKTVIISCTLSSGNIENNYVHWYQQRPGRAPTTVIFDDDKRPDGVPDRFSGSIDRSSNSASLTISGLQTEDEADYYCHSYVSSFNVFGGGTKLTVLSGGGGSGGGGSGGGGSEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSS

SEQ ID NO: 111 - protein, 561 aa, Unknown
  bi-specific molecule
METDTLLLWVLLVWIPGSTGEVKLVESGGGLVQPGRPLKLSCAASGFTFSNYGMAWVRQAPKKGLEWVATISTGDDNTYYRDSVKGRFTISRDNAKNTQYLQMDSLRSEDTATYYCATHNYESTIFDYWGQGVMVTVSSGGGGSGGGGSGGGGSDIAITQSPASMTASPGERVTITCRASSSVSYMHWYQQKPGASPKPWIYETSKLASGVPDRFSGSGSGTSYSLTINNMEAEDAATYYCQQWNYPSMYTFGAGTKLELKRSDPTTTPAPRPPTPAPTIASQPLSLRPEACRPAAGGAVHTRGLDFACDSGGGGSDFMLTQPHSVSESPGKTVIISCTLSSGNIENNYVHWYQQRPGRAPTTVIFDDDKRPDGVPDRFSGSIDRSSNSASLTISGLQTEDEADYYCHSYVSSFNVFGGGTKLTVLSGGGGSGGGGSGGGGSEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSS

SEQ ID NO: 112 - protein, 561 aa, Unknown
  bi-specific molecule
METDTLLLWVLLVWIPGSTGEVQLVESGGGFVQPGGSLKLSCAASGFTFSNYGMAWVRQAPKKGLEWVASISSGRGNTYYRDSVKGRFTISRDNAKNTQYLQMDSLRSEDTATYYCVTHNYESTIFDYWGQGVMVTVSSGGGGSGGGGSGGGGSEIVLTQSPTTMTASPGEKVTIICRASSSVSYMHWYQQKPGASPKPWIYETSKLASGVPDRFSGSGSGTSYSLTFNNMEAEDAATYYCQQWNYPSMYTFGAGTKLELKRSDPTTTPAPRPPTPAPTIASQPLSLRPEACRPAAGGAVHTRGLDFACDSGGGGSDFMLTQPHSVSESPGKTVIISCTLSSGNIENNYVHWYQQRPGRAPTTVIFDDDKRPDGVPDRFSGSIDRSSNSASLTISGLQTEDEADYYCHSYVSSFNVFGGGTKLTVLSGGGGSGGGGSGGGGSEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSS

SEQ ID NO: 113 - protein, 6 aa, Unknown
  linker
SGGGGS

SEQ ID NO: 114 - protein, 119 aa, Unknown
  VH
EVKLVESGGGLVQPGRSLKLSCAASGFIFSNYAMAWVRQAPTKGLEWVASVNSGGANTYYRDSVKGRFTISRDNAKNTQYLHIDSLRSEDTATYYCATHNYESTVFDYWGQGAMVTVSS

SEQ ID NO: 115 - protein, 107 aa, Unknown
  VL
EIVLTQSPTTMTASPGEKVTITCRASSSVSYMHWYQQKPGASPKPWIYETSKLASGVPDRFSGSGSGTSYSLTINNMEAEDAATYYCQQWNYPSMYTFGAGTKLELK

SEQ ID NO: 116 - protein, 119 aa, Unknown
  VH
EVQLVESGGDLVQPGRSLKLSCAASGFTFSNYGMAWVRQAPTKGLEWVASVSNGGGNTYYRDSVKGRFTISRDNAKNTQYLQMDSLRSEDTATYYCVTHNYESTVFDYWGQGAMVTVSS

SEQ ID NO: 117 - protein, 107 aa, Unknown
  VL
EIVLTQSPTTMTASPGEKVTITCRASSSVSYMHWYQQKPGASPKPWIYETSKLASGVPDRFSGSGSGTSYSLTINNMEAEDAATYYCQQWNYPSMYTFGPGTKLELK

SEQ ID NO: 118 - protein, 112 aa, Unknown
  VH
QVQLKESGPGLVQPSETLSLTCTVSGFSLTNYGINWFRQPPGKGLEWMGTIWNDGGTDYNSALKSRLTISRDTSKSQVFLKMNSLQTEDTAIYFCARGMEAWGQGASVTVSS

SEQ ID NO: 119 - protein, 106 aa, Unknown
  VL
DIQLTQSPSLLSASVGDRVTLSCKGSQNINNYLAWYQQKLGEAPKLLIYNTNSLQTGIPSRFSGSGSGTDYTLTISSLQPEDFATYFCLQHNSGYTFGAGTKLEIK

SEQ ID NO: 120 - protein, 119 aa, Unknown
  VH
EVQLVESGGGFVQPGRSLKLSCAASGFTFSNYAMAWVRQAPTKGLEWVASINSGGGDSYYRDSVKGRFTISRDNAKNTQYLQMDSLRSEDTATYYCATHNYGSTIFDYWGQGAMVTVSS

SEQ ID NO: 121 - protein, 107 aa, Unknown
  VL
EIILTQSPTTMTASPGEKVTITCRASSSVSYMHWYQQKPGASPKPWIYETSKLASGVPDRFSGSGSGTSYSLTINNMEAEDAATYYCQQWNYPSMYTFGAGTKLELK

SEQ ID NO: 122 - protein, 112 aa, Unknown
  VH
EVQLVESGPGLVQPSETLSLTCTVSGFSLTSHGVNWFRQPPGKGLEWMGTIWNDGGTDYNSALKSRLSIIRDTSKGQVFLKMNSLQTEDTAIYFCARGLAYWGQGTLVTVSS

SEQ ID NO: 123 - protein, 107 aa, Unknown
  VL
DIQMTQSPASLSASLGETVNIECLASEDIYSDLAWYQQKPGKSPQLLIYNANSLQNGVPSRFSGSGSGTQYSLKINSLQSEDVATYFCQQYNNYPWTFGGGTKLEIK

SEQ ID NO: 124 - protein, 112 aa, Unknown
  VH
QVNLQESGPGLVQPSETLSLTCTVSGFSLTNYGINWFRQPPGKGLEWMGTIWNDGGTDYNSALKSRLSISRDTSKSQVFLKMNSLQTEDTAIYFCARGMDAWGQGASVTVSS

SEQ ID NO: 125 - protein, 107 aa, Unknown
  VL
DIQMTQSPASLSASLGETVSIECLASEDIYNNLAWYQQKPGKSPQLLIYYANSLQDGVPSRFSGSGSGTQYSLKINSLQSEDVATYFCQQYNNYPFTFGSGTKLELK

SEQ ID NO: 126 - protein, 119 aa, Unknown
  VH
EVQLVESGGGLVQPGRSLKLSCAASGFTFSNYAMAWVRQAPTKGLEWVASISSGGGDTYYRDSVKGRFTISRDNSKNTQYLQMDSLRSEDTATYYCATHNYESTVLITWGQGASVTVSS

SEQ ID NO: 127 - protein, 107 aa, Unknown
  VL
EIVLTQSPTTTAASPGERVTITCRASSSVSYMHWYQQKPGASPKPWIYETSKLASGVPDRFSGSGSGTSYSLTINNMEAEDAATYYCQQWNYPSMYTFGAGTKLEIK

SEQ ID NO: 128 - protein, 112 aa, Unknown
  VH
EVQLVESGPGLVQPSETLSLTCTVSGFSLTSYGVNWFRQPPGKGLEWMGTIWNDGGTDYNSALKSRLSISRDTSKSQVFLKMNSLQTEDTAIYFCARGFEYWGQGTLVTVSS

SEQ ID NO: 129 - protein, 107 aa, Unknown
  VL
DIVMTQSPKSMSISVGDRVTMNCKASQNVGKNIAWYQQKPGQSPKLLIYYASNRYTGVPDRFTGSGSGTDFTLTITNMQTEDLAVYYCLQYNYNPWTFGGGTKLELK

SEQ ID NO: 130 - protein, 125 aa, Unknown
  VH
EVQLVESGGGLVQPGRSLKLSCAASGFTFSNYGMAWVRQAPTKGLEWVASINTSGGSTYYRDSVKGRFTISRDNAKSTLYLQMDSLRSEDTATYYCATIYYHDGSYYYAGWFAYWGQGTLVTVSS

SEQ ID NO: 131 - protein, 106 aa, Unknown
  VL
DIVLTQSPALAVSPGERVSISCRASESVSTLMHWYQQKLGQQPKLLIYGASNLESGVPARFSGSGSGTDFTLNIDPVEADDIATYFCQQSWNGPWTFGGGTKLELK

SEQ ID NO: 132 - protein, 112 aa, Unknown
  VH
QVNLQESGPGLVRPSETLSLTCTVSGFSLISYGVNWFRQPPGKGLEWMGTIWNDGGTDYNSALKSRLSISRDTSKSQVFLKMNSLQTEDTAIYFCARGFEHWGQGVMVTVSS

SEQ ID NO: 133 - protein, 107 aa, Unknown
  VL
DIQMTQSPASLSASLGETVSIECLTSEDISNDSAWYQQKSGKSPQLLIYAASRLQDGVPSRFSGSGSGTRYSLKISGMQPEDEADYFCQQSYKYPLTFGSGTKLEIK

SEQ ID NO: 134 - protein, 119 aa, Unknown
  VH
EVKLVESGGGLVQPGRSLKLSCAASGFTFSNYGMAWVRQAPTKGLEWVATISNGGGNTYYRDSVKGRFTISRDNAKNTQYLQMDSLRSEDTATYYCVAHNYESTVFDYWGQGAMVTVSS

SEQ ID NO: 135 - protein, 107 aa, Unknown
  VL
EIVLTQSPTPMTASPGEKVTITCRASSSVSYMHWYQQKPGASPKPWIYETSKLASGVPDRFSGSGSGTSYSLTINNMEAEDAATYYCQQWNYPSMYTFGAGTKLELK

SEQ ID NO: 136 - protein, 119 aa, Unknown
  VH
EVKLVESGGGLVQPGRSLKLSCTASGFTFSNYATAWVRQAPTKGLEWVASIITGVGNTYYRDSVKGRFTISRDNARNTQYLQMDSLRSEDTATYYCEHITTSPPFLITWGQGSWSQSPQ

SEQ ID NO: 137 - protein, 107 aa, Unknown
  VL
EIVLTQSPTTTTASPGERVTITCRASSSVSYMHWYQQKPGASPKPWIYETSKLASGVPDRFSGSGSGTSYSLTINNMEAEDAATYYCQQWNYPSMYTFGAGTKLEIK

SEQ ID NO: 138 - protein, 119 aa, Unknown
  VH
EVQLVESGGGLVQPGRSLKLSCAASRFTFSNYAMAWVRQAPTKGLEWVASISSGAGNTYYRDSVKGRFTISRDNAKNTQYLQMGSLRSEDTATYYCVTHNYESTVFDYWGQGASVTVSS

SEQ ID NO: 139 - protein, 107 aa, Unknown
  VL
ETVLTQSPTTTPASPGEKVTITCRASSSVSYMHWYQQKSGASPKLWIYDTSKLASGVPNRFSGSGSGTSYSLTINNMEAEDAATYYCQQWNYPSMYTFGPGTKLEIK

SEQ ID NO: 140 - protein, 112 aa, Unknown
  VH
QVNLQESGPGLVQPSETLSLTCTVSGFSLTNYGINWFRQPPGKGLEWMGTIWNDGGTDYNSALKSRLTISRDTSKSQVFLKMNSLQTEDTAIYFCARGMEAWGQGASVTVSS

SEQ ID NO: 141 - protein, 107 aa, Unknown
  VL
DIRMTQSPASLSASLGETVSIECLASEDIYKNLAWYQQKPGKSPQLLIYYASSLQDGVPSRFSGSGSGTQYSLKINSLESEDAATYFCLQDSEYPLTFGSGTKLEIK

SEQ ID NO: 142 - protein, 119 aa, Unknown
  VH
EVQLVESGGGLVQPGRSLKLSCAASGFTFSNYAMAWVRQAPTKGLEWVASINSGGGNTYYRDSVKGRFTISRDNAKNTQYLQMDSLRSEDTATYYCATHNYESTVFDYWGQGVMVTVSS

SEQ ID NO: 143 - protein, 107 aa, Unknown
  VL
EIVLTQSPTSMTASPGERVTITCRASSSVSYMHWYQQKPGASPKPWIYETSKLASGVPDRFSGSGSGTSYSLTINNMEAEDAATYYCQQWNYPSMYTFGAGTKLEIK

SEQ ID NO: 144 - protein, 119 aa, Unknown
  VH
EVKLVESGGGLVQPGRSLKLSCAASGFTFSNYAMAWVRQAPTKGLEWVASIHSGGGNTYYRDSVKGRFTISRDNAKNTQYLQMDSLRSEDTATYYCVTHNYESTVFDYWGQGAMVTVSS

SEQ ID NO: 145 - protein, 107 aa, Unknown
  VL
EIVLTQSPTTMTASPGEKVTITCRASSSVSYMHWYQQKPGASPKPWIYETSKLASGVPDRFSGSGSGTSYSLTINNMEAEDAATYYCQQWNYPSMYTFGAGTKLEIK

SEQ ID NO: 146 - protein, 119 aa, Unknown
  VH
EVKLVESGGGLVQPGRSLKLSCAASGFTFSNYGMAWVRQAPTKGLEWVATISNGGGNTYYRDSVKGRFTISRDNAKNTQYLQMDSLRSEDTATYYCVTHNYESTVFDYWGQGAMVTVSS

SEQ ID NO: 147 - protein, 3 aa, Unknown
  LCDR2
DTS

SEQ ID NO: 148 - protein, 119 aa, Unknown
  VH
EVQLVESGGDLVQPGRSLKLSCAASGFTFSNYAMAWVRQAPTKGLEWVASISNGGGNSFYRDSVKGRFTISRDNAKNTQYLQMDSLRSEDTATYYCATHNYESTVFDYWGRGVMVTVSS

SEQ ID NO: 149 - protein, 107 aa, Unknown
  VL
EIVLTQSPTTMTASPGEKVTITCRASSSVSYMHWYQQKPGASPKPWIYETSKLASGVPDRFSGSGSGTSYSLTINNMEAEDAATYYCQQWNYPSMYTFGPGTKLEIK

SEQ ID NO: 150 - protein, 119 aa, Unknown
  VH
EVQLVESGGGLVQPGRSLKLSCAASGFTFSNYGMAWVRQAPTKGLEWVASIGSGGSDTYYRDSVKGRFTISRDNAKNTQYLQMDSLRSEDTATYYCATHNYESTVFDYWGQGVMVTVSS

SEQ ID NO: 151 - protein, 107 aa, Unknown
  VL
DTVLTQSPTSMTASPGERVTITCRASSSVSYMHWYQQKPGASPKPWIYETSKLASGVPDRFSGSGSGTSYSLTINNMEAEDAATYYCQQWNYPSMYTFGAGTKLEIK

SEQ ID NO: 152 - protein, 119 aa, Unknown
  VH
EVKLVESGGGLVQPGRSLKLSCAASGFTFSTYAMAWVRQAPTKGLEWVASINSGGGDTYYRDSVKGRFTISRDNTKNTQYLQMDSLRSEDTATYYCATHNYESTVFDYWGQGVMVTVSS

SEQ ID NO: 153 - protein, 107 aa, Unknown
  VL
EIVLTQSPTTMTASPGEKVTITCRASSSVSYMHWYQQKPGASPKPWIYETSKLASGVPDRFSGSGSGTSYSLTFNNMEAEDAATYYCQQWNYPSMYTFGAGTKLELK

SEQ ID NO: 154 - protein, 119 aa, Unknown
  VH
EVQLVESGGGLVQPGRSLKLSCAASGFTFSNYAMAWVRQAPTKGLEWVASISSGGGDTYYRDSVKGRFTISRDNSKNTQYLQMDSLRSEDTATYYCATHNYESTVFDYWGQGASVTVSS

SEQ ID NO: 155 - protein, 6 aa, Unknown
  LCDR1
EDIYKN

SEQ ID NO: 156 - protein, 119 aa, Unknown
  VH
EVQLVESGGGLVQPGRSLKLSCAASGFIFGNYAMAWVRQAPTKGLEWVASVNSGGGNTYYRDSVKGRFTISRDNAKNTQYLHIDGLRSEDTATYYCATHNYESTVFDYWGQGASVTVSS

SEQ ID NO: 157 - protein, 9 aa, Unknown
  LCDR3
LQDSEYPLT

SEQ ID NO: 158 - protein, 243 aa, Unknown
  scFv sequence
EVKLVESGGGLVQPGRSLKLSCAASGFIFSNYAMAWVRQAPTKGLEWVASVNSGGANTYYRDSVKGRFTISRDNAKNTQYLHIDSLRSEDTATYYCATHNYESTVFDYWGQGAMVTVSSGGGGSGGGGSSGGGGSEIVLTQSPTTMTASPGEKVTITCRASSSVSYMHWYQQKPGASPKPWIYETSKLASGVPDRFSGSGSGTSYSLTINNMEAEDAATYYCQQWNYPSMYTFGAGTKLELKR

SEQ ID NO: 159 - protein, 242 aa, Unknown
  scFv sequence
EVQLVESGGDLVQPGRSLKLSCAASGFTFSNYGMAWVRQAPTKGLEWVASVSNGGGNTYYRDSVKGRFTISRDNAKNTQYLQMDSLRSEDTATYYCVTHNYESTVFDYWGQGAMVTVSSGGGGSGGGGSGGGGSEIVLTQSPTTMTASPGEKVTITCRASSSVSYMHWYQQKPGASPKPWIYETSKLASGVPDRFSGSGSGTSYSLTINNMEAEDAATYYCQQWNYPSMYTFGPGTKLELKR

SEQ ID NO: 160 - protein, 234 aa, Unknown
  scFv sequence
QVQLKESGPGLVQPSETLSLTCTVSGFSLTNYGINWFRQPPGKGLEWMGTIWNDGGTDYNSALKSRLTISRDTSKSQVFLKMNSLQTEDTAIYFCARGMEAWGQGASVTVSSGGGGSGGGGSGGGGSDIQLTQSPSLLSASVGDRVTLSCKGSQNINNYLAWYQQKLGEAPKLLIYNTNSLQTGIPSRFSGSGSGTDYTLTISSLQPEDFATYFCLQHNSGYTFGAGTKLEIKR

SEQ ID NO: 161 - protein, 242 aa, Unknown
  scFv sequence
EVQLVESGGGFVQPGRSLKLSCAASGFTFSNYAMAWVRQAPTKGLEWVASINSGGGDSYYRDSVKGRFTISRDNAKNTQYLQMDSLRSEDTATYYCATHNYGSTIFDYWGQGAMVTVSSGGGGSGGGGSGGGGSEIILTQSPTTMTASPGEKVTITCRASSSVSYMHWYQQKPGASPKPWIYETSKLASGVPDRFSGSGSGTSYSLTINNMEAEDAATYYCQQWNYPSMYTFGAGTKLELKR

SEQ ID NO: 162 - protein, 235 aa, Unknown
  scFv sequence
EVQLVESGPGLVQPSETLSLTCTVSGFSLTSHGVNWFRQPPGKGLEWMGTIWNDGGTDYNSALKSRLSIIRDTSKGQVFLKMNSLQTEDTAIYFCARGLAYWGQGTLVTVSSGGGGSGGGGSGGGGSDIQMTQSPASLSASLGETVNIECLASEDIYSDLAWYQQKPGKSPQLLIYNANSLQNGVPSRFSGSGSGTQYSLKINSLQSEDVATYFCQQYNNYPWTFGGGTKLEIKR

SEQ ID NO: 163 - protein, 235 aa, Unknown
  scFv sequence
QVNLQESGPGLVQPSETLSLTCTVSGFSLTNYGINWFRQPPGKGLEWMGTIWNDGGTDYNSALKSRLSISRDTSKSQVFLKMNSLQTEDTAIYFCARGMDAWGQGASVTVSSGGGGSGGGGSGGGGSDIQMTQSPASLSASLGETVSIECLASEDIYNNLAWYQQKPGKSPQLLIYYANSLQDGVPSRFSGSGSGTQYSLKINSLQSEDVATYFCQQYNNYPFTFGSGTKLELKR

SEQ ID NO: 164 - protein, 242 aa, Unknown
  scFv sequence
EVQLVESGGGLVQPGRSLKLSCAASGFTFSNYAMAWVRQAPTKGLEWVASISSGGGDTYYRDSVKGRFTISRDNSKNTQYLQMDSLRSEDTATYYCATHNYESTVLITWGQGASVTVSSGGGGSGGGGSGGGGSEIVLTQSPTTTAASPGERVTITCRASSSVSYMHWYQQKPGASPKPWIYETSKLASGVPDRFSGSGSGTSYSLTINNMEAEDAATYYCQQWNYPSMYTFGAGTKLEIKR

SEQ ID NO: 165 - protein, 235 aa, Unknown
  scFv sequence
EVQLVESGPGLVQPSETLSLTCTVSGFSLTSYGVNWFRQPPGKGLEWMGTIWNDGGTDYNSALKSRLSISRDTSKSQVFLKMNSLQTEDTAIYFCARGFEYWGQGTLVTVSSGGGGSGGGGSGGGGSDIVMTQSPKSMSISVGDRVTMNCKASQNVGKNIAWYQQKPGQSPKLLIYYASNRYTGVPDRFTGSGSGTDFTLTITNMQTEDLAVYYCLQYNYNPWTFGGGTKLELKR

SEQ ID NO: 166 - protein, 247 aa, Unknown
  scFv sequence
EVQLVESGGGLVQPGRSLKLSCAASGFTFSNYGMAWVRQAPTKGLEWVASINTSGGSTYYRDSVKGRFTISRDNAKSTLYLQMDSLRSEDTATYYCATIYYHDGSYYYAGWFAYWGQGTLVTVSSGGGGSGGGGSGGGGSDIVLTQSPALAVSPGERVSISCRASESVSTLMHWYQQKLGQQPKLLIYGASNLESGVPARFSGSGSGTDFTLNIDPVEADDIATYFCQQSWNGPWTFGGGTKLELKR

SEQ ID NO: 167 - protein, 235 aa, Unknown
  scFv sequence
QVNLQESGPGLVRPSETLSLTCTVSGFSLISYGVNWFRQPPGKGLEWMGTIWNDGGTDYNSALKSRLSISRDTSKSQVFLKMNSLQTEDTAIYFCARGFEHWGQGVMVTVSSGGGGSGGGGSGGGGSDIQMTQSPASLSASLGETVSIECLTSEDISNDSAWYQQKSGKSPQLLIYAASRLQDGVPSRFSGSGSGTRYSLKISGMQPEDEADYFCQQSYKYPLTFGSGTKLEIKR

SEQ ID NO: 168 - protein, 242 aa, Unknown
  scFv sequence
EVKLVESGGGLVQPGRSLKLSCAASGFTFSNYGMAWVRQAPTKGLEWVATISNGGGNTYYRDSVKGRFTISRDNAKNTQYLQMDSLRSEDTATYYCVAHNYESTVFDYWGQGAMVTVSSGGGGSGGGGSGGGGSEIVLTQSPTPMTASPGEKVTITCRASSSVSYMHWYQQKPGASPKPWIYETSKLASGVPDRFSGSGSGTSYSLTINNMEAEDAATYYCQQWNYPSMYTFGAGTKLELKR

SEQ ID NO: 169 - protein, 242 aa, Unknown
  scFv sequence
EVKLVESGGGLVQPGRSLKLSCTASGFTFSNYATAWVRQAPTKGLEWVASIITGVGNTYYRDSVKGRFTISRDNARNTQYLQMDSLRSEDTATYYCEHITTSPPFLITWGQGSWSQSPQGGGGSGGGGSGGGGSEIVLTQSPTTTTASPGERVTITCRASSSVSYMHWYQQKPGASPKPWIYETSKLASGVPDRFSGSGSGTSYSLTINNMEAEDAATYYCQQWNYPSMYTFGAGTKLEIKR

SEQ ID NO: 170 - protein, 242 aa, Unknown
  scFv sequence
EVQLVESGGGLVQPGRSLKLSCAASRFTFSNYAMAWVRQAPTKGLEWVASISSGAGNTYYRDSVKGRFTISRDNAKNTQYLQMGSLRSEDTATYYCVTHNYESTVFDYWGQGASVTVSSGGGGSGGGGSGGGGSETVLTQSPTTTPASPGEKVTITCRASSSVSYMHWYQQKSGASPKLWIYDTSKLASGVPNRFSGSGSGTSYSLTINNMEAEDAATYYCQQWNYPSMYTFGPGTKLEIKR

SEQ ID NO: 171 - protein, 235 aa, Unknown
  scFv sequence
QVNLQESGPGLVQPSETLSLTCTVSGFSLTNYGINWFRQPPGKGLEWMGTIWNDGGTDYNSALKSRLTISRDTSKSQVFLKMNSLQTEDTAIYFCARGMEAWGQGASVTVSSGGGGSGGGGSGGGGSDIRMTQSPASLSASLGETVSIECLASEDIYKNLAWYQQKPGKSPQLLIYYASSLQDGVPSRFSGSGSGTQYSLKINSLESEDAATYFCLQDSEYPLTFGSGTKLEIKR

SEQ ID NO: 172 - protein, 242 aa, Unknown
  scFv sequence
EVQLVESGGGLVQPGRSLKLSCAASGFTFSNYAMAWVRQAPTKGLEWVASINSGGGNTYYRDSVKGRFTISRDNAKNTQYLQMDSLRSEDTATYYCATHNYESTVFDYWGQGVMVTVSSGGGGSGGGGSGGGGSEIVLTQSPTSMTASPGERVTITCRASSSVSYMHWYQQKPGASPKPWIYETSKLASGVPDRFSGSGSGTSYSLTINNMEAEDAATYYCQQWNYPSMYTFGAGTKLEIKR

SEQ ID NO: 173 - protein, 242 aa, Unknown
  scFv sequence
EVKLVESGGGLVQPGRSLKLSCAASGFTFSNYAMAWVRQAPTKGLEWVASIHSGGGNTYYRDSVKGRFTISRDNAKNTQYLQMDSLRSEDTATYYCVTHNYESTVFDYWGQGAMVTVSSGGGGSGGGGSGGGGSEIVLTQSPTTMTASPGEKVTITCRASSSVSYMHWYQQKPGASPKPWIYETSKLASGVPDRFSGSGSGTSYSLTINNMEAEDAATYYCQQWNYPSMYTFGAGTKLEIKR

SEQ ID NO: 174 - protein, 242 aa, Unknown
  scFv sequence
EVKLVESGGGLVQPGRSLKLSCAASGFTFSNYGMAWVRQAPTKGLEWVATISNGGGNTYYRDSVKGRFTISRDNAKNTQYLQMDSLRSEDTATYYCVTHNYESTVFDYWGQGAMVTVSSGGGGSGGGGSGGGGSEIVLTQSPTPMTASPGEKVTITCRASSSVSYMHWYQQKPGASPKPWIYETSKLASGVPDRFSGSGSGTSYSLTINNMEAEDAATYYCQQWNYPSMYTFGAGTKLELKR

SEQ ID NO: 175 - protein, 242 aa, Unknown
  scFv
EVQLVESGGDLVQPGRSLKLSCAASGFTFSNYAMAWVRQAPTKGLEWVASISNGGGNSFYRDSVKGRFTISRDNAKNTQYLQMDSLRSEDTATYYCATHNYESTVFDYWGRGVMVTVSSGGGGSGGGGSGGGGSEIVLTQSPTTMTASPGEKVTITCRASSSVSYMHWYQQKPGASPKPWIYETSKLASGVPDRFSGSGSGTSYSLTINNMEAEDAATYYCQQWNYPSMYTFGPGTKLEIKR

SEQ ID NO: 176 - protein, 242 aa, Unknown
  scFv sequence
EVQLVESGGGLVQPGRSLKLSCAASGFTFSNYGMAWVRQAPTKGLEWVASIGSGGSDTYYRDSVKGRFTISRDNAKNTQYLQMDSLRSEDTATYYCATHNYESTVFDYWGQGVMVTVSSGGGGSGGGGSGGGGSDTVLTQSPTSMTASPGERVTITCRASSSVSYMHWYQQKPGASPKPWIYETSKLASGVPDRFSGSGSGTSYSLTINNMEAEDAATYYCQQWNYPSMYTFGAGTKLEIKR

SEQ ID NO: 177 - protein, 242 aa, Unknown
  scFv sequence
EVKLVESGGGLVQPGRSLKLSCAASGFTFSTYAMAWVRQAPTKGLEWVASINSGGGDTYYRDSVKGRFTISRDNTKNTQYLQMDSLRSEDTATYYCATHNYESTVFDYWGQGVMVTVSSGGGGSGGGGSGGGGSEIVLTQSPTTMTASPGEKVTITCRASSSVSYMHWYQQKPGASPKPWIYETSKLASGVPDRFSGSGSGTSYSLTFNNMEAEDAATYYCQQWNYPSMYTFGAGTKLELKR

SEQ ID NO: 178 - protein, 242 aa, Unknown
  scFv sequence
EVQLVESGGGLVQPGRSLKLSCAASGFTFSNYAMAWVRQAPTKGLEWVASISSGGGDTYYRDSVKGRFTISRDNSKNTQYLQMDSLRSEDTATYYCATHNYESTVFDYWGQGASVTVSSGGGGSGGGGSGGGGSEIVLTQSPTTTAASPGERVTITCRASSSVSYMHWYQQKPGASPKPWIYETSKLASGVPDRFSGSGSGTSYSLTINNMEAEDAATYYCQQWNYPSMYTFGAGTKLEIKR

SEQ ID NO: 179 - protein, 242 aa, Unknown
  scFv sequence
EVQLVESGGGLVQPGRSLKLSCAASGFIFGNYAMAWVRQAPTKGLEWVASVNSGGGNTYYRDSVKGRFTISRDNAKNTQYLHIDGLRSEDTATYYCATHNYESTVFDYWGQGASVTVSSGGGGSGGGGSGGGGSEIILTQSPTTMTASPGEKVTITCRASSSVSYMHWYQQKPGASPKPWIYETSKLASGVPDRFSGSGSGTSYSLTINNMEAEDAATYYCQQWNYPSMYTFGAGTKLELKR

SEQ ID NO: 180 - protein, 8 aa, Unknown
  HCDR2
ISSGAGNT

SEQ ID NO: 181 - protein, 8 aa, Unknown
  HCDR2
INSGGGNT

SEQ ID NO: 182 - protein, 8 aa, Unknown
  HCDR2
IHSGGGNT

SEQ ID NO: 183 - protein, 8 aa, Unknown
  HCDR2
ISNGGGNS

SEQ ID NO: 184 - protein, 8 aa, Unknown
  HCDR2
IGSGGSDT

SEQ ID NO: 185 - protein, 8 aa, Unknown
  HCDR1
GFTFSTYA

SEQ ID NO: 186 - protein, 8 aa, Unknown
  HCDR2
INSGGGDT

SEQ ID NO: 187 - protein, 8 aa, Unknown
  HCDR1
GFIFGNYA

SEQ ID NO: 188 - protein, 8 aa, Unknown
  HCDR2
VNSGGGNT

(19) United States
(12) Patent Application Publication
(10) Pub. No.: US 2024/0158500 A1
(43) Pub. Date: May 16, 2024

(54) CD160 Binding Domain

(71) Applicant: UCL BUSINESS LTD, London (GB)

(72) Inventors: Samir Agrawal, London (GB); Martin Pulé, London (GB); Francesco Nannini, London (GB)

(21) Appl. No.: 18/550,739

(22) PCT Filed: Nov. 4, 2021
(86) PCT No.: PCT/GB21/52862

§ 371 Date: Sep. 15, 2023

(30) Foreign Application Priority Data
Mar. 17, 2021 (GB) 2103706.4

(51) Int. Cl. C07K 16/28; A61K 39/00; A61P 35/00; C07K 14/725; C12N 15/86

(52) U.S. Cl. CPC C07K 16/2803; A61K 39/4611; A61K 39/4631; A61K 39/4643; A61P 35/00; C07K 14/7051; C07K 16/2809; C07K 16/2815; C07K 16/2818; C07K 16/2878; C12N 15/86; C07K 2317/24; C07K 2317/31; C07K 2317/622; C07K 2319/03; C07K 2319/30; C12N 2740/13043

(57) ABSTRACT

The present invention provides CD160 binding domains. The invention also relates to antibodies or fragments thereof, chimeric antigen receptors (CARs) and bi-specific T cell engagers (BiTEs) which comprise such binding domains.

### FIELD OF THE INVENTION

The present invention relates to CD160 binding domains. The invention also relates to antibodies or fragments thereof, chimeric antigen receptors (CARs) and bi-specific T cell engagers (BiTEs) which comprise such CD160 binding domains. Modalities targeting CD160 may be useful in the treatment of cancerous diseases such as NK lymphoma, TCRγδ lymphoma, chronic lymphocytic leukaemia (CLL), or hairy cell leukaemia, for example.

### BACKGROUND TO THE INVENTION

Chronic lymphocytic leukaemia (CLL) is the most common haematological malignancy and remains an incurable cancer. As such it provides an unsolved clinical problem.

CLL lacks a satisfactory immunotherapeutic. For example, anti-CD20 therapeutics provide limited efficacy because CD20 is not densely expressed in CLL. Anti-CD19 based immunotherapeutics target the entire B-cell compartment and are far from routinely used in CLL. CD52 targeting with Alemtuzumab is effective in CLL but comes at the cost of depleting the entire lymphoid compartment, increasing susceptibility to opportunistic infection.

There is thus a need for improved therapeutic approaches to treat CLL.

CD160 is a 27 KDa Ig-like activating natural killer (NK) cell receptor expressed on most circulating Natural Killer (NK) cells, most peripheral blood TCRγδ lymphocytes, all small intestinal intraepithelial T lymphocytes and on a subset of circulating CD8+ and CD4+TCRαβ T cell, but typically not on B cells.

CD160 was initially thought to be GPI-anchored, but a type I trans-membrane isoform has been described with a cytoplasmic tail capable of activating the ERK1/2 pathway. CD160 interacts with classical and non-classical MHC class I molecules. CD160 also interacts with the receptor Herpesvirus entry mediator (HVEM), a prominent member of the TNF receptor superfamily. Engagement of CD160 activates NK cells.

CD160 is aberrantly expressed by B-cell chronic lymphocytic leukemia (B-CLL) and Hairy Cell Leukaemia (HCL). CD160 is expressed on NK Lymphomas and TCRγδ lymphomas. Notably these latter malignancies are rare but lack satisfactory treatments and are associated with poor prognosis. Farren et al. (as above) determined that CD160 was expressed in 98% (590 of 600) of CLL cases and 100% (32 of 32) of hairy cell leukemia (HCL) cases. CD160 was present in only approximately 15% of other B-cell lymphoproliferative disease (B-LPD) cases. As such CD160 may have utility as a therapeutic target, particularly for lymphoid malignancies. Outside of haematopoietic system, CD160 has also been shown to be expressed on newly forming vessels, but not healthy vessels. CD160 is expressed on endothelial cells of neoangiogenic microvasculature within tumours. In non-lymphoid tissue, expression of CD160 appears restricted to neo-angiogenesis; CD160 is expressed by growing but not quiescent endothelial cells (Fons et al., 2006) and blocking CD160 results in anti-angiogenic effects.

### SUMMARY OF THE INVENTION

The present inventors have surprisingly demonstrated that CD160 binding domains comprising the complementarity determining regions (CDRs) described herein are capable of binding to their target CD160 with high affinity. These CD160 binders may be useful in antigen binding entities such as antibodies, chimeric antigen receptors (CARs) and bi-specific T cell engagers (BiTEs).

Thus, in a first aspect, the present invention provides a CD160 binding domain, wherein the CD160 binding domain comprises:
(i) complementarity determining regions (CDRs) 1-3 comprising SEQ ID NOs 1-3; and CDRs 4-6 comprising SEQ ID NOs 4-6;
(ii) CDRs 1-3 comprising SEQ ID NOs 7-9; and CDRs 4-6 comprising SEQ ID NOs 4-6;
(iii) CDRs 1-3 comprising SEQ ID NOs 10-12; and CDRs 4-6 comprising SEQ ID NOs 13-15;
(iv) CDRs 1-3 comprising SEQ ID NOs 7, 16 and 17; and CDRs 4-6 comprising SEQ ID NOs 4-6;
(v) CDRs 1-3 comprising SEQ ID NOs 18, 11 and 19; and CDRs 4-6 comprising SEQ ID NOs 20-22;
(vi) CDRs 1-3 comprising SEQ ID NOs 23-25; and CDRs 4-6 comprising SEQ ID NOs 4-6;
(vii) CDRs 1-3 comprising SEQ ID NOs 26, 11 and 27; and CDRs 4-6 comprising SEQ ID NOs 28-30;
(viii) CDRs 1-3 comprising SEQ ID NOs 18, 11 and 31; and CDRs 4-6 comprising SEQ ID NOs 13, 32 and 33;
(xi) CDRs 1-3 comprising SEQ ID NOs 23, 34 and 35; and CDRs 4-6 comprising SEQ ID NOs 4-6;
(x) CDRs 1-3 comprising SEQ ID NOs 10, 11 and 36; and CDRs 4-6 comprising SEQ ID NOs 37, 14 and 38;
(xi) CDRs 1-3 comprising SEQ ID NOs 7, 39 and 40; and CDRs 4-6 comprising SEQ ID NOs 41-43;
(xii) CDRs 1-3 comprising SEQ ID NOs 7, 44 and 45; and CDRs 4-6 comprising SEQ ID NOs 4-6;
(xiii) CDRs 1-3 comprising SEQ ID NOs 46, 11 and 48; and CDRs 4-6 comprising SEQ ID NOs 49-51;
(xiv) CDRs 1-3 comprising SEQ ID NOs 7, 52 and 53; and CDRs 4-6 comprising SEQ ID NOs 4-6; or
(xv) CDRs 1-3 comprising SEQ ID NOs 23, 54 and 55; and CDRs 4-6 comprising SEQ ID NOs 4-6;
optionally wherein one or more of the CDRs comprises one, two or three amino acid mutations.

In one embodiment, the CDRs 1-3 may comprise or consist of SEQ ID NOs 1-3; and the CDRs 4-6 may comprise or consist of SEQ ID NOs 4-6.

In one embodiment, the CDRs 1-3 may comprise or consist of SEQ ID NOs 7-9; and the CDRs 4-6 may comprise or consist of SEQ ID NOs 4-6.

In one embodiment, the CDRs 1-3 may comprise or consist of SEQ ID NOs 10-12; and the CDRs 4-6 may comprise or consist of SEQ ID NOs 13-15.

In one embodiment, the CDRs 1-3 may comprise or consist of SEQ ID NOs 7, 16 and 17; and the CDRs 4-6 may comprise or consist of SEQ ID NOs 4-6.

In one embodiment, the CDRs 1-3 may comprise or consist of SEQ ID NOs 18, 11 and 19; and the CDRs 4-6 may comprise or consist of SEQ ID NOs 20-22.

In one embodiment, the CDRs 1-3 may comprise or consist of SEQ ID NOs 23-25; and the CDRs 4-6 may comprise or consist of SEQ ID NOs 4-6.

In one embodiment, the CDRs 1-3 may comprise or consist of SEQ ID NOs 26, 11 and 27; and the CDRs 4-6 may comprise or consist of SEQ ID NOs 28-30.

In one embodiment, the CDRs 1-3 may comprise or consist of SEQ ID NOs 18, 11 and 31; and the CDRs 4-6 may comprise or consist of SEQ ID NOs 13, 32 and 33.

In one embodiment, the CDRs 1-3 may comprise or consist of SEQ ID NOs 23, 34 and 35; and the CDRs 4-6 may comprise or consist of SEQ ID NOs 4-6.

In one embodiment, the CDRs 1-3 may comprise or consist of SEQ ID NOs 10, 11 and 36; and the CDRs 4-6 may comprise or consist of SEQ ID NOs 37, 14 and 38.

In one embodiment, the CDRs 1-3 may comprise or consist of SEQ ID NOs 7, 39 and 40; and the CDRs 4-6 may comprise or consist of SEQ ID NOs 41-43.

In one embodiment, the CDRs 1-3 may comprise or consist of SEQ ID NOs 7, 44 and 45; and the CDRs 4-6 may comprise or consist of SEQ ID NOs 4-6.

In one embodiment, the CDRs 1-3 may comprise or consist of SEQ ID NOs 46, 11 and 48; and the CDRs 4-6 may comprise or consist of SEQ ID NOs 49-51.

In one embodiment, the CDRs 1-3 may comprise or consist of SEQ ID NOs 7, 52 and 53; and the CDRs 4-6 may comprise or consist of SEQ ID NOs 4-6.

In one embodiment, the CDRs 1-3 may comprise or consist of SEQ ID NOs 23, 54 and 55; and the CDRs 4-6 may comprise or consist of SEQ ID NOs 4-6.

Suitably, the CD160 binding domain may comprise a heavy variable region (VH) and a light variable region (VL) comprising a combination of CDR 1-3 and CDRs 4-6 as defined above.

The CD160 binding domain may comprise a VH region having the sequence shown as SEQ ID NO: 114 or a variant having at least 80% sequence identity thereto; and a VL region having the sequence shown as SEQ ID NO: 115 or a variant of having at least 80% sequence identity thereto.

The CD160 binding domain may comprise a VH region having the sequence shown as SEQ ID NO: 116 or a variant having at least 80% sequence identity thereto; and a VL region having the sequence shown as SEQ ID NO: 117 or a variant of having at least 80% sequence identity thereto.

The CD160 binding domain may comprise a VH region having the sequence shown as SEQ ID NO: 56 or a variant having at least 80% sequence identity thereto; and a VL region having the sequence shown as SEQ ID NO: 57 or a variant of having at least 80% sequence identity thereto.

The CD160 binding domain may comprise a VH region having the sequence shown as SEQ ID NO: 118 or a variant having at least 80% sequence identity thereto; and a VL region having the sequence shown as SEQ ID NO: 119 or a variant of having at least 80% sequence identity thereto.

The CD160 binding domain may comprise a VH region having the sequence shown as SEQ ID NO: 120 or a variant having at least 80% sequence identity thereto; and a VL region having the sequence shown as SEQ ID NO: 121 or a variant of having at least 80% sequence identity thereto.

The CD160 binding domain may comprise a VH region having the sequence shown as SEQ ID NO: 124 or a variant having at least 80% sequence identity thereto; and a VL region having the sequence shown as SEQ ID NO: 125 or a variant of having at least 80% sequence identity thereto.

The CD160 binding domain may comprise a VH region having the sequence shown as SEQ ID NO: 126 or a variant having at least 80% sequence identity thereto; and a VL region having the sequence shown as SEQ ID NO: 127 or a variant of having at least 80% sequence identity thereto.

The CD160 binding domain may comprise a VH region having the sequence shown as SEQ ID NO: 130 or a variant having at least 80% sequence identity thereto; and a VL region having the sequence shown as SEQ ID NO: 131 or a variant of having at least 80% sequence identity thereto.

The CD160 binding domain may comprise the sequence shown as SEQ ID NO: 158 or a variant thereof at least 80% sequence identity thereto.

The CD160 binding domain may comprise the sequence shown as SEQ ID NO: 159 or a variant thereof at least 80% sequence identity thereto.

The CD160 binding domain may comprise the sequence shown as SEQ ID NO: 62 or a variant thereof having at least 80% sequence identity.

The CD160 binding domain may comprise the sequence shown as SEQ ID NO: 160 or a variant thereof at least 80% sequence identity thereto.

The CD160 binding domain may comprise the sequence shown as SEQ ID NO: 161 or a variant thereof at least 80% sequence identity thereto.

The CD160 binding domain may comprise the sequence shown as SEQ ID NO: 163 or a variant thereof at least 80% sequence identity thereto.

The CD160 binding domain may comprise the sequence shown as SEQ ID NO: 164 or a variant thereof at least 80% sequence identity thereto.

The CD160 binding domain may comprise the sequence shown as SEQ ID NO: 166 or a variant thereof at least 80% sequence identity thereto.

In a second aspect, the present invention provides an antibody or antigen-binding fragment thereof comprising the CD160 antigen binding domain according to the first aspect of the invention.

The antibody or fragment thereof may be a scFv, a monoclonal antibody or fragment thereof, or a humanized antibody or fragment thereof.

In a third aspect, the present invention provides an antibody conjugate comprising the antibody or fragment thereof according to the second aspect of the invention.

In a fourth aspect, the present invention provides a chimeric antigen receptor (CAR) comprising a CD160 binding domain according to the first aspect of the invention.

The CAR may comprise a transmembrane domain which comprises the sequence comprises the sequence selected from the group comprising SEQ ID NO: 65 or SEQ ID NO: 66, or a variant thereof having at least 80% sequence identity. The CD160 binding domain and the transmembrane domain may be connected by a spacer.

Suitably, the spacer may comprise one of the following: an IgG1 Fc domain; an IgG1 hinge; an IgG1 hinge-CD8 stalk; or a CD8 stalk. The spacer may comprise the sequence selected from the group comprising SEQ ID NO: 74, SEQ ID NO: 75, SEQ ID NO: 76, SEQ ID NO: 77 or SEQ ID NO: 78 or a variant thereof having at least 80% sequence identity.

The CAR may comprise an intracellular T cell signalling domain. Suitably, the intracellular T cell signalling domain may comprise one or more of the following endodomains: CD28 endodomain; 41BB endodomain; OX40 endodomain and CD3-Zeta endodomain. Suitably, the intracellular T cell signalling domain comprises all of the following endodomains: CD28 endodomain; 41BB endodomain; OX40 and CD3-Zeta endodomain.

The CAR may comprise the sequence selected from the group comprising SEQ ID NO: 79 or SEQ ID NO: 80 or SEQ ID NO: 81, or a variant thereof which has at least 80% sequence identity thereto but retains the capacity to i) bind CD160 and ii) induce T cell signalling.

In a fifth aspect, the present invention provides a T cell activator molecule which is a bi-specific molecule comprising:
(i) a first domain which comprises a CD160 binding domain according to the first aspect of the invention; and
(ii) a second domain capable of activating a T cell.

The second domain may activate a T cell by binding CD3 on the T cell surface. Suitably, the second domain may comprise a CD3-specific antibody or part thereof. The second domain may comprise the sequence selected from the group comprising SEQ ID NO: 90, SEQ ID NO: 97 or SEQ ID NO: 104, or a variant thereof which has at least 80% sequence identity and binds CD3.

The first and second binding domains may be connected by a spacer. Suitably, the spacer may comprise an IgG1 hinge or a CD8 stalk.

The bi-specific molecule may comprise the sequence selected from the group comprising SEQ ID NO: 110, SEQ ID NO: 111 or SEQ ID NO: 112 or a variant thereof which has at least 80% sequence identity but retains the capacity to i) bind CD160 and ii) activate a T cell.

In a sixth aspect, the present invention provides a polynucleotide comprising a nucleic acid sequence encoding a CD160 binding domain according to the first aspect of the invention, an antibody or fragment thereof according to the second aspect of the invention, a CAR according to the fourth aspect of the invention or a bi-specific molecule according to the fifth aspect of the invention.

In a seventh aspect, the present invention provides a vector which comprises a polynucleotide according to the sixth aspect of the invention.

In an eighth aspect, the present invention provides a cell which comprises a CAR according to the fourth aspect of the invention.

The cell may be a T cell or a natural killer (NK) cell.

In a ninth aspect, the present invention provides a cell comprising the polynucleotide according to the sixth aspect of the invention or a vector according to the seventh aspect of the invention.

In a tenth aspect, the present invention provides a method for making a cell according to the eighth or ninth aspect of the invention, which comprises the step of introducing a polynucleotide according to the sixth aspect of the invention or a vector according to the seventh aspect of the invention into said cell.

In an eleventh aspect, the present invention provides a pharmaceutical composition which comprises a CD160 binding domain according to the first aspect of the invention, an antibody or fragment thereof according to the second aspect of the invention, or an antibody conjugate according to the third aspect of the invention, or a bi-specific molecule according to the fifth aspect of the invention, or a vector according to the seventh aspect of the invention, or a cell according to the eighth or ninth aspect of the invention, together with a pharmaceutically acceptable carrier, diluent or excipient.

In an twelfth aspect, the present invention provides a method for treating a disease which comprises the step of administering a CD160 binding domain according to the first aspect of the invention, an antibody or fragment thereof according to the second aspect of the invention, or an antibody conjugate according to the third aspect of the invention, or a bi-specific molecule according to the fifth aspect of the invention, or a vector according to the seventh aspect of the invention, or a cell according to the eighth or ninth aspect of the invention or a pharmaceutical composition according to the eleventh aspect of the invention to a subject.

In an thirteenth aspect, the present invention provides use of a CD160 binding domain according to the first aspect of the invention, an antibody or fragment thereof according to the second aspect of the invention, or an antibody conjugate according to the third aspect of the invention, or a bi-specific molecule according to the fifth aspect of the invention, or a vector according to the seventh aspect of the invention, or a cell according to the eighth or ninth aspect of the invention or a pharmaceutical composition according to the eleventh aspect of the invention in the manufacture of a medicament for treating a disease.

In an fourteenth aspect, the present invention provides a CD160 binding domain according to the first aspect of the invention, an antibody or fragment thereof according to the second aspect of the invention, or an antibody conjugate according to the third aspect of the invention, or a bi-specific molecule according to the fifth aspect of the invention, or a vector according to the seventh aspect of the invention, or a cell according to the eighth or ninth aspect of the invention or a pharmaceutical composition according to the eleventh aspect of the invention for use as a medicament in the treatment of a disease.

Suitably, the disease to be treated may be cancer. The cancer may be selected from: NK lymphoma, TCRγδ lymphoma, chronic lymphocytic leukaemia, or hairy cell leukaemia. The disease may be associated with neoangiogenesis.

### BRIEF DESCRIPTION OF THE FIGURES

FIG. 1—Alignment of the 29 primers for the heavy variable genes (VH) to the IMGT rat germline database. Each primer was aligned against the reference rat germline database IMGT; The degree of homology to the rat's germline sequence was represented as a color-coded scale. The number of ambiguous nucleotides (Y, R, W, S, K and M) and Locked Nucleic Acids (LNAs) was reported for each primer.

FIG. 2—NGS analysis of the VH and VK genes in naïve rats. Chord diagram representation of the V and J genes frequency and their respective pairing; analysis of the 5′RACE-amplified cDNA(left) and primer-set amplified (right) for VH [A] and VK [B]. The inner circle colours represent the joining regions paired with each variable gene family.

FIG. 3—Genetic vaccination with pVAC2 plasmid expressing human CD160. [A] pVAC2 plasmid map for the expression of codon optimized human CD160 GPI-anchor protein. The plasmid contains a CpG-free bacterial promoter (EM7) and a CpG-free Zeocin-resistance gene (Sh-ΔCpG). The EF1α gene promoter and its polyadenylation signal (EF1pAN) combined with SV40 enhancer drive expression of the transgene in mammalian cells. [B] pVAC2 expression in transiently transfected 293T cells. Cells were stained for flow cytometry with anti-CD160 antibody using non-transfected cells (NT) as a negative control. [C] Flow cytometry analysis of the rat serum pre- and post-vaccination against 293T cells NT or transfected to express human CD160. Cells were run unstained (red line) stained with anti-rat alone (blue line), pre-vaccination serum (orange line) and post vaccination serum (green line).

FIG. 4—SupT1 cells overexpressing human CD160. CD160 was cloned into the retroviral vector SFG and SupT1 cells expressing high levels of CD160 were generated. Flow cytometry histograms of SupT1 NT (red) and SupT1 SFG.huCD160 (blue) stained with anti-CD160 (BY55)-PE.

FIG. 5—Amplification of rat's VH and VK for the assembly as scFv library. [A] PCR amplification of the 29 primers for the VH (top) and 39 for the VL (bottom). [B] Assembly as scFv library by overlap extension PCR in naïve and CD160-vaccinated rats. 800 bp product indicate full-length scFv. [C] Serial gel extraction of the full-length scFv assembled by overlap extension PCR (1), the 800 bp product first extraction from the agarose gel (2) showing the carry over of the shorter product, the full-length product is extracted from the agarose gel a second time (3) to minimize the presence of the 600 bp band. Second extraction of the shorter band is shown (4) to highlight the separation between the two products.

FIG. 6—Biopanning of the scFv phage display library on Streptactin™ magnetic beads. [A] Schematic representation of the biopanning process employing the streptag-streptactin system. CD160 fused with Twin streptag was capture directly from cell supernatant on the surface of Streptactin™ magnetic beads. Phages were incubated with the antigen' coated beads and bound phages were subsequently recovered by eluting with a 30 mM biotin solution and used to amplify the library for the next biopanning round.[B] The process of coating and elution of the beads throughout the biopanning process was assessed by flow cytometry analysis. These were stained during the several steps of the procedure (coating, blocking with 3% BSA and elution) using commercial anti CD160-PE. [C] Enrichment of the phage library for CD160 was determined after three rounds of biopanning on beads. The bacterial library from the different rounds of selection was induced to express the soluble scFv fragment in the supernatant. This was used to stain SupT1 cells overexpressing human CD160, due to the presence of a myc-tag at the C-terminus of productive scFvs we were able to detect binding using anti myc antibody. 2×TY media+anti myc-tag-DL549® and the commercial anti-CD160-PE Ab were used as controls. [D] The 15 individual bacterial colonies identified in the third round of biopanning carrying unique combination of CDR1,2 and 3 were screened by against SupT1 CD160 positive and negative cells (NT).

FIG. 7—Analysis of CD160 reagents used for panning. [A] Supernatant used for panning the library was purified via the Fc region of the protein, Size exclusion chromatography highlighted the purity of the protein and showed a homogenous peak at the expected elution volume. [B] Western blotting analysis (using BY55 as CD160 specific antibody). The purified protein could be detected at the same molecular weight as a corresponding band within the supernatant used for antibody selection. [C] Differential scanning fluorimetry (DSF), showed that the protein obtained was stable and had an apparent melting temperature above that observed with commercially obtained protein. [D] Three sources of CD160 protein, commercially sourced, unpurified from supernatant and purified were used as coating antigens in ELISA experiments. Comparable EC50 profiles were obtained with all the antigen tested using BY55-huIgG1 as primary antibody for the detection.

FIG. 8—Characterization of the monoclonal scFv identified in the biopanning. [A] Alignment to the IMGT database of Rattus Norvegicus germline variable sequence showed rearrange sequences originated from family 2 and 5 of the HV genes. We identified five different CDR3H in which three were represented in multiple clones and three occurring only once. [B] Flow cytometry analysis of the five selected CDR3 expressed secreted as chimeric scFv fused with mIgG2aFc. Supernatant from transfected 293T cells was used to stain SupT1 negative and SupT1 CD160 positive cells; the binding was detected with a secondary anti-mouseIgGFc-PE and anti CD160-PE (BY55) was used as positive control. [C] The affinity of these five scFv was determined using Biacore T200 technology. Individual scFvs were captured on a CM5 chip and CD160 was injected as analyte at 5 different concentration (3.70 nM, 11.11 nM, 33.33, nM, 100 nM and 300 nM) in a single-cycle kinetic study. The double-reference subtracted sensorgrams fitted with the 1:1 Langmuir binding model; kinetic and model fitting data are reported in the table.

FIG. 9—Functional assays as chimeric antigen receptor. Healthy donors PBMCs transduced with lentivirus to express the five different scFvs fused in a third generation (CD28-OX40ζ) CAR structure and tested in a cytotoxic assay. T-cells were incubated with SupT1 CD160 positive and negative cells (NT) at different effector to target ratio (1:2, 1:4 and 1:8). All the five CARs demonstrated excellent killing at both 24 h and 72 and [A] Total cytokine production assessed at 72 h showed high levels of interferon-gamma (IFN-γ) and IL-2 [B] with no background on target-negative cells.

FIG. 10—Functional assays for bi-specific T cell engagers. Effectors:Targets ratio 1:2 (targets SupT1 NT, SupT1_CD160 and primary CLL cells). Readout at 48 h (n=3). Activated healthy donors T-cells were incubated with target cells in the presence of HEK293T supernatant containing the 5 different anti-CD160 BiTEs or No BiTE (negative control). Cytotoxicity and interferon-gamma (IFN-γ) production were assessed after 48 h showing that all the five BiTEs molecules teste have good degree of killing and cytokine production against cells overexpressing the CD160 molecule with no background on target-negative cells.

FIG. 11—CD160 staining of GPI and TM isoforms. 8 different anti-CD160 clones were tested against the GPI and TM isoform of CD160.

FIG. 12—Immunohistochemistry (IHC) staining optimization. Four anti-CD160 antibodies (1 mg/ml) were tested on FFPE sections of healthy tonsils.

FIG. 13—NK and γδ T cell lines. A) Characteristics of NK and γδ cell lines. B) Chipcytometry. PB9 has been used to stain DERL7 and YT cell lines.

FIG. 14—Number of CD160 molecules on NK and γδ T cell lines. A) γδ and NK cell lines were stained with the 2 directly conjugated anti-CD160 antibodies (PB6 and PB9), commercial BY55 was used for comparison. B) The number of molecules per cell was obtained using QuantiBrite-PE beads.

FIG. 15—Anti-CD160 CAR T cell generation showing transduction efficiency of second generation 4-1BBzeta CAR-T cell constructs.

FIG. 16—Anti-CD160 CAR-T cell testing—cytotoxicity assay showing killing against NK and gamma-delta tumour cell lines.

### DETAILED DESCRIPTION OF THE INVENTION

### Binding Affinity

Binding affinity is typically described as the strength of the binding interaction between two molecules, (e.g. between a receptor and its ligand or an antibody and its cognate antigen). Binding affinity may be defined by determining the equilibrium dissociation constant (KD), which is used to measure the strengths of molecular interactions. A lower KD value indicates a higher binding affinity, and vice versa a higher KD value indicates a lower binding affinity.

In one aspect the present invention provides a CD160 binding domain which has a binding affinity KD value of 1.1 nM or lower.

In one aspect the present invention provides a CD160 binding domain which has a binding affinity KD value of 0.2 nM or lower.

The binding affinity KD value may be less than 1.1 nM, for example less than 1, 0.9, 0.8, 0.7, 0.6, 0.5, 0.4, 0.3, 0.2 or 0.1 nM. Preferably, the CD160 binding domain may have a binding affinity KD value of less than 0.2 nM. For example, the binding affinity KD value may be less than 0.15 nM. Suitably, the binding affinity KD value may be less than 0.1 nM.

Suitably, the CD160 binding domain may have a binding affinity KD value in the range of 1.1 nM to 0.1 pM (i.e. 1100 pM to 0.1 pM). Preferably, the binding affinity KD value may be in the range of 0.2 nM to 0.1 pM (i.e 200 pM to 0.1 pM). The binding affinity KD value may be in the range of 0.15 nM to 0.1 pM (i.e 150 pM to 0.1 pM). The binding affinity KD value may be less than 150, 140, 130, 120, 110, 100, 50, 25, 20, 15, 10, 5, 4, 3, 2 or 1 pM. The binding affinity KD value may be less than 0.9, 0.8, 0.7, 0.6, 0.5, 0.4, 0.3, 0.2 or 0.1 pM.

Suitably, the binding affinity KD value may be less than 0.1 pM. The CD160 binding domain may have a binding affinity KD value in the range of 1.1 nM to 0.01 pM (i.e. 1100 pM to 0.01 pM). Preferably, the binding affinity KD value may be in the range of 0.2 nM to 0.01 pM (i.e 200 pM to 0.01 pM). The binding affinity KD value may be in the range of 0.15 nM to 0.01 pM (i.e 150 pM to 0.01 pM). The binding affinity KD value may be less than 0.09, 0.08, 0.07, 0.06, 0.05, 0.04, 0.03, 0.02, 0.015 or 0.01 pM.

A quantitative assessment or measurement of binding affinity (e.g. establishing a KD value) may be determined or measured using methods know in the art, such as by surface plasmon resonance, for example by using the Biacore® system. In addition to the equilibrium dissociation constant (KD), the association rate constant (Ka (1/Ms)), and the dissociation rate constant (Kd (1/s)) may also be determined.

Surface Plasmon Resonance (SPR) experiments may be performed with a Biacore T200, for example.

Methods for determining binding specificity include, but are not limited to, ELISA, western blot, immunohistochemistry, flow cytometry, Förster resonance energy transfer (FRET), phage display libraries, yeast two-hybrid screens, co-immunoprecipitation, bimolecular fluorescence complementation and tandem affinity purification. Binding affinity can also be determined using methods such as fluorescence quenching, isothermal titration calorimetry.

### CD160 Binding Domain

The major form of CD160 is a glycosylphosphatidylinositol (GPI)-anchored cell surface molecule with a single immunoglobulin (Ig)-like domain that is weakly homologous to the KIR2DL4 receptor. This Ig-like domain contains six cysteine residues allowing the formation of multimers tightly linked by disulphide bonds. In contrast to the GPI-anchored isoform, a transmembrane CD160 isoform has been reported to be selectively expressed by activated PB-NK cells and NK cell lines. In addition, CD160 has been demonstrated to be involved in neo-angiogenesis.

The present inventors now provide antigen-binding domains capable of binding to CD160 with high affinity.

The binding domains described herein are able to specifically bind to CD160. The CD160 binding domain may be capable of selectively binding to the glycosylphosphatidylinositol (GPI) and/or transmembrane (TM) and/or soluble isoforms of CD160. Suitably, the CD160 binding domain may be capable of selectively binding to the GPI and TM isoforms of CD160.

Suitably, a heavy chain variable (VH) region may comprise the CDR1, CDR2 and CDR3 sequences. Suitably, a light chain variable (VL) region may comprise the CDR4, CDRS and CDR6 sequences. CDR4, CDRS and CDR6 may be presented or re-numbered as CDR1, CDR2 and CDR3 of the VL region, respectively. Therefore, the CDRs of the VH region may be numbered as CDR1, CDR2 and CDR3, and the CDRs of the VL region may be numbered as CDR1, CDR2 and CDR3.

Thus, a CD160 binding domain may comprise:
a VH region having CDRs with the following numbering:
   CDR1
   CDR2
   CDR3; and
a VL region having CDRs with the following numbering:
   CDR1
   CDR2
   CDR3.

In one embodiment, the CD160 binding domain may comprise a VH region comprising SEQ ID NOs 1-3; and a VL region comprising SEQ ID NOs 4-6.

In one embodiment, the CD160 binding domain may comprise a VH region comprising SEQ ID NOs 7-9; and a VL region comprising SEQ ID NOs 4-6.

In one embodiment, the CD160 binding domain may comprise a VH region comprising SEQ ID NOs 10-12; and a VL region comprising SEQ ID NOs 13-15.

In one embodiment, the CD160 binding domain may comprise a VH region comprising SEQ ID NOs 7, 16 and 17; and a VL region comprising SEQ ID NOs 4-6.

In one embodiment, the CD160 binding domain may comprise a VH region comprising SEQ

ID NOs 18, 11 and 19; and a VL region comprising SEQ ID NOs 20-22.

In one embodiment, the CD160 binding domain may comprise a VH region comprising SEQ ID NOs 23-25; and a VL region comprising SEQ ID NOs 4-6.

In one embodiment, the CD160 binding domain may comprise a VH region comprising SEQ ID NOs 26, 11 and 27; and a VL region comprising SEQ ID NOs 28-30.

In one embodiment, the CD160 binding domain may comprise a VH region comprising SEQ ID NOs 18, 11 and 31; and a VL region comprising SEQ ID NOs 13, 32 and 33.

In one embodiment, the CD160 binding domain may comprise a VH region comprising SEQ ID NOs 23, 34 and 35; and a VL region comprising SEQ ID NOs 4-6.

In one embodiment, the CD160 binding domain may comprise a VH region comprising SEQ ID NOs 10, 11 and 36; and a VL region comprising SEQ ID NOs 37, 14 and 38.

In one embodiment, the CD160 binding domain may comprise a VH region comprising SEQ ID NOs 7, 39 and 40; and a VL region comprising SEQ ID NOs 41-43.

In one embodiment, the CD160 binding domain may comprise a VH region comprising SEQ

ID NOs 7, 44 and 45; and a VL region comprising SEQ ID NOs 4-6.

In one embodiment, the CD160 binding domain may comprise a VH region comprising SEQ ID NOs 46, 11 and 48; and a VL region comprising SEQ ID NOs 49-51.

In one embodiment, the CD160 binding domain may comprise a VH region comprising SEQ ID NOs 7, 52 and 53; and a VL region comprising SEQ ID NOs 4-6.

In one embodiment, the CD160 binding domain may comprise a VH region comprising SEQ ID NOs 23, 54 and 55; and a VL region comprising SEQ ID NOs 4-6.

The CD160 binding domain may comprise a VH region having the sequence shown as SEQ ID NO: 56 or a variant having at least 80%, 85%, 90%, 95%, 98% or 99% sequence identity thereto; and a VL region having the sequence shown as SEQ ID NO: 57 or a variant of having at least 80%, 85%, 90%, 95%, 98% or 99% sequence identity thereto. Suitably, the CD160 binding domain may comprise a VH region having the sequence shown as SEQ ID NO: 56; and a VL region having the sequence shown as SEQ ID NO: 57.

### VH (SEQ ID NO: 56):

QVNLQESGPGLVQPSETLSLICTVSGFSLTSYGVYWVRQPPGKGLEWMGRIWNDGGIDYNPALKSRLS ISRDTSKSQVFLKLNSLQTEDTAIYFCARGWTHWGQGTLVTVSS

### VL (SEQ ID NO: 57):

DIQMTQSPASLSASLGETVSIECLASEDIYNNLAWYQQKPGKSPQLLIYYASSLQDGVPSRFSGSGSG TQYSLKINSLESEDVATYFCLQDSEYPPTFGGGTKLELK

The CD160 binding domain may comprise a VH region having the sequence shown as SEQ ID NO: 58 or a variant having at least 80%, 85%, 90%, 95%, 98% or 99% sequence identity thereto; and a VL region having the sequence shown as SEQ ID NO: 59 or a variant of having at least 80%, 85%, 90%, 95%, 98% or 99% sequence identity thereto. Suitably, the CD160 binding domain may comprise a VH region having the sequence shown as SEQ ID NO: 58; and a VL region having the sequence shown as SEQ ID NO: 59.

VH (SEQ ID NO: 58):
EVKLVESGGGLVQPGRPLKLSCAASGFTFSNYGMAWVRQAPKKGLEWVA
TISTGDDNTYYRDSVKGRFTISRDNAKNTQYLQMDSLRSEDTATYYCAT
HNYESTIFDYWGQGVMVTVSS
VL (SEQ ID NO: 59):
DIAITQSPASMTASPGERVTITCRASSSVSYMHWYQQKPGASPKPWIYE
TSKLASGVPDRFSGSGSGTSYSLTINNMEAEDAATYYCQQWNYPSMYTF
GAGTKLELK

The CD160 binding domain may comprise a VH region having the sequence shown as SEQ ID NO: 60 or a variant having at least 80%, 85%, 90%, 95%, 98% or 99% sequence identity thereto; and a VL region having the sequence shown as SEQ ID NO: 61 or a variant of having at least 80%, 85%, 90%, 95%, 98% or 99% sequence identity thereto. Suitably, the CD160 binding domain may comprise a VH region having the sequence shown as SEQ ID NO: 60; and a VL region having the sequence shown as SEQ ID NO: 61.

VH (SEQ ID NO: 60):
EVQLVESGGGFVQPGGSLKLSCAASGFTESNYGMAWVRQAPKKGLEWVA
SISSGRGNTYYRDSVKGRFTISRDNAKNTQYLQMDSLRSEDTATYYCVT
HNYESTIFDYWGQGVMVTVSS
VL (SEQ ID NO: 61):
EIVLTQSPTTMTASPGEKVTIICRASSSVSYMHWYQQKPGASPKPWIYE
TSKLASGVPDRFSGSGSGTSYSLTENNMEAEDAATYYCQQWNYPSMYTF
GAGTKLELK

Suitably, the CD160 binding domain comprises the amino acid sequence shown as SEQ ID NO: 62 or a variant thereof having at least 80%, 85%, 90%, 95%, 98% or 99% sequence identity thereto.

SEQ ID NO: 62
QVNLQESGPGLVQPSETLSLTCTVSGFSLTSYGVYWVRQPPGKGLEWMG
RIWNDGGTDYNPALKSRLSISRDTSKSQVFLKLNSLQTEDTAIYFCARG
WTHWGQGTLVTVSSGGGGSGGGGSGGGGSDIQMTQSPASLSASLGETVS
IECLASEDIYNNLAWYQQKPGKSPQLLIYYASSLQDGVPSRFSGSGSGT
QYSLKINSLESEDVATYFCLQDSEYPPTFGGGTKLELKR

Suitably, the CD160 binding domain comprises the amino acid sequence shown as SEQ ID NO: 63 or a variant thereof at least 80%, 85%, 90%, 95%, 98% or 99% sequence identity thereto.

SEQ ID NO: 63
EVKLVESGGGLVQPGRPLKLSCAASGFTFSNYGMAWVRQAPKKGLEWVA
TISTGDDNTYYRDSVKGRFTISRDNAKNTQYLQMDSLRSEDTATYYCAT
HNYESTIFDYWGQGVMVTVSSGGGGSGGGGSGGGGSDIAITQSPASMTA
SPGERVTITCRASSSVSYMHWYQQKPGASPKPWIYETSKLASGVPDRFS
GSGSGTSYSLTINNMEAEDAATYYCQQWNYPSMYTFGAGTKLELKR

Suitably, the CD160 binding domain comprises the amino acid sequence shown as SEQ ID NO: 64 or a variant thereof at least 80%, 85%, 90%, 95%, 98% or 99% sequence identity thereto.

SEQ ID NO: 64
EVQLVESGGGFVQPGGSLKLSCAASGFTFSNYGMAWVRQAPKKGLEWVA
SISSGRGNTYYRDSVKGRFTISRDNAKNTQYLQMDSLRSEDTATYYCVT
HNYESTIFDYWGQGVMVTVSSGGGGSGGGGSGGGGSEIVLTQSPTTMTA
SPGEKVTIICRASSSVSYMHWYQQKPGASPKPWIYETSKLASGVPDRFS
GSGSGTSYSLTENNMEAEDAATYYCQQWNYPSMYTFGAGTKLELKR

In one aspect, the CD160 binding domain may comprise the set of six CDR sequences listed for one of the clones identified in Table 1 below. Suitably, one or more of the CDRs may comprise comprises one, two or three amino acid mutations.

TABLE 1

| Clone | HCDR1 | HCDR2 | HCDR3 | LCDR1 | LCDR2 | LCDR3 |
|---|---|---|---|---|---|---|
| 1 | GFIFSNYA | VNSGGANT | ATHNYESTVFDY | SSVSY | ETS | QQWNYPSMYT |
| | (SEQ ID NO: 1) | (SEQ ID | (SEQ ID NO: 3) | (SEQ ID NO: 4) | (SEQ ID | (SEQ ID NO: 6) |
| | | NO: 2) | | | NO: 5) | |
| 2 | GFTFSNYG | VSNGGGNT | VTHNYESTVFDY | SSVSY | ETS | QQWNYPSMYT |
| | (SEQ ID NO: 7) | (SEQ ID | (SEQ ID NO: 9) | (SEQ ID NO: 4) | (SEQ ID | (SEQ ID NO: 6) |
| | | NO: 8) | | | NO: 5) | |
| 3 | GFSLTSYG | IWNDGGT | ARGWTH | EDIYNN | YAS | LQDSEYPPT |
| | (SEQ ID NO: 10) | (SEQ ID | (SEQ ID NO: 12) | (SEQ ID NO: 13) | (SEQ ID | (SEQ ID NO: 15) |
| | | NO: 11) | | | NO: 14) | |
| 4 | GFTFSNYG | ISTGDDNT | ATHNYESTIFDY | SSVSY | ETS | QQWNYPSMYT |
| | (SEQ ID NO: 7) | (SEQ ID | (SEQ ID NO: 17) | (SEQ ID NO: 4) | (SEQ ID | (SEQ ID NO: 6) |
| | | NO: 16) | | | NO: 5) | |
| 5 | GFSLTNYG | IWNDGGT | ARGMEA | QNINNY | INTN | LQHNSGYT |
| | (SEQ ID NO: 18) | (SEQ ID | (SEQ ID NO: 19) | (SEQ ID NO: 20) | (SEQ ID | (SEQ ID NO: 22) |
| | | NO: 11) | | | NO: 21) | |
| 6 | GFTFSNYA | INSGGGDS | ATHNYGSTIFDY | SSVSY | ETS | QQWNYPSMYT |
| | (SEQ ID NO: 23) | (SEQ ID | (SEQ ID NO: 25) | (SEQ ID NO: 4) | (SEQ ID | (SEQ ID NO: 6) |
| | | NO: 24) | | | NO: 5) | |
| 7 | GFSLTSHG | IWNDGGT | ARGLAY | EDIYSD | NAN | QQYNNYPWT |
| | (SEQ ID NO: 26) | (SEQ ID | (SEQ ID NO: 27) | (SEQ ID NO: 28) | (SEQ ID | (SEQ ID NO: 30) |
| | | NO: 11) | | | NO: 29) | |
| 8 | GFSLTNYG | IWNDGGT | ARGMDA | EDIYNN | YAN | QQYNNYPFT |
| | (SEQ ID NO: 18) | (SEQ ID | (SEQ ID NO: 31) | (SEQ ID NO: 13) | (SEQ ID | (SEQ ID NO: 33) |
| | | NO: 11) | | | NO: 32) | |
| 9 | GFTFSNYA | ISSGGGDT | ATHNYESTVLIT | SSVSY | ETS | QQWNYPSMYT |
| | (SEQ ID NO: 23) | (SEQ ID | (SEQ ID NO: 35) | (SEQ ID NO: 4) | (SEQ ID | (SEQ ID NO: 6) |
| | | NO: 34) | | | NO: 5) | |
| 10 | GFSLTSYG | IWNDGGT | ARGFEY | QNVGKN | YAS | LQYNYNPWT |
| | (SEQ ID NO: 10) | (SEQ ID | (SEQ ID NO: 36) | (SEQ ID NO: 37) | (SEQ ID | (SEQ ID NO: 38) |
| | | NO: 11) | | | NO: 14) | |
| 11 | GFTFSNYG | INTSGGST | ATIYYHDGSYYYA | ESVSTL | GAS | QQSWNGPWT |
| | (SEQ ID NO: 7) | (SEQ ID | GWFAY | (SEQ ID NO: 41) | (SEQ ID | (SEQ ID NO: 43) |
| | | NO: 39) | (SEQ ID NO: 40) | | NO: 42) | |
| 12 | GFTFSNYG | ISSGRGNT | VTHNYESTIFDY | SSVSY | ETS | QQWNYPSMYT |
| | (SEQ ID NO: 7) | (SEQ ID | (SEQ ID NO: 45) | (SEQ ID NO: 4) | (SEQ ID | (SEQ ID NO: 6) |
| | | NO: 44) | | | NO: 5) | |
| 16 | GFSLISYG | IWNDGGT | ARGFEH | EDISND | AAS | QQSYKYPLT |
| | (SEQ ID NO: 46) | (SEQ ID | (SEQ ID NO: 48) | (SEQ ID NO: 49) | (SEQ ID | (SEQ ID NO: 51) |
| | | NO: 11) | | | NO: 50) | |
| 19 | GFTFSNYG | ISNGGGNT | VAHNYESTVFDY | SSVSY | ETS | QQWNYPSMYT |
| | (SEQ ID NO: 7) | (SEQ ID | (SEQ ID NO: 53) | (SEQ ID NO: 4) | (SEQ ID | (SEQ ID NO: 6) |
| | | NO: 52) | | | NO: 5) | |
| 20 | GFTFSNYA | IITGVGNT | EHITTSPPFLIT | SSVSY | ETS | QQWNYPSMYT |
| | (SEQ ID NO: 23) | (SEQ ID | (SEQ ID NO: 55) | (SEQ ID NO: 4) | (SEQ ID | (SEQ ID NO: 6) |
| | | NO: 54) | | | NO: 5) | |

In one aspect, the CD160 binding domain may comprise the set of heavy chain CDR1-3 sequences listed for one of the clones identified in Table 2 below. Suitably, one or more of the CDRs may comprise comprises one, two or three amino acid mutations.

In one embodiment, the CD160 binding domain may comprise a VH region comprising SEQ ID NOs 47, 180 and 9; and a VL region comprising SEQ ID NOs 4, 147 and 6.

In one embodiment, the CD160 binding domain may comprise a VH region comprising SEQ ID NOs 18, 11 and 19; and a VL region comprising SEQ ID NOs 155, 14, and 157.

In one embodiment, the CD160 binding domain may comprise a VH region comprising SEQ ID NOs 23, 181 and 3; and a VL region comprising SEQ ID NOs 4-6.

In one embodiment, the CD160 binding domain may comprise a VH region comprising SEQ ID NOs 23, 182 and 9; and a VL region comprising SEQ ID NOs 4-6.

In one embodiment, the CD160 binding domain may comprise a VH region comprising SEQ ID NOs 7, 52 and 9; and a VL region comprising SEQ ID NOs 4-6.

In one embodiment, the CD160 binding domain may comprise a VH region comprising SEQ ID NOs 23, 183 and 3; and a VL region comprising SEQ ID NOs 4-6.

In one embodiment, the CD160 binding domain may comprise a VH region comprising SEQ ID NOs 7, 184 and 3; and a VL region comprising SEQ ID NOs 4-6.

In one embodiment, the CD160 binding domain may comprise a VH region comprising SEQ ID NOs 185, 186 and 3; and a VL region comprising SEQ ID NOs 4-6.

In one embodiment, the CD160 binding domain may comprise a VH region comprising SEQ ID NOs 23, 34 and 3; and a VL region comprising SEQ ID NOs 4-6.

In one embodiment, the CD160 binding domain may comprise a VH region comprising SEQ ID NOs 187, 188 and 3; and a VL region comprising SEQ ID NOs 4-6.

TABLE 2

| Clone | HCDR1 | HCDR2 | HCDR3 | LCDR1 | LCDR2 | LCDR3 |
|---|---|---|---|---|---|---|
| C68 | RFTFSNYA | ISSGAGNT | VTHNYESTVFDY | SSVSY | DTS | QQWNYPSMYT |
| | (SEQ ID NO: 47) | (SEQ ID | (SEQ ID NO: 9) | (SEQ ID | (SEQ ID | (SEQ ID NO: 6) |
| | | NO: 180) | | NO: 4) | NO: 147) | |
| C92 | GFSLTNYG | IWNDGGT | ARGMEA | EDIYKN | YAS | LQDSEYPLT |
| | (SEQ ID NO: 18) | (SEQ ID | (SEQ ID NO: 19) | (SEQ ID | (SEQ ID NO: 14) | (SEQ ID |
| | | NO: 11) | | NO: 155) | | NO: 157) |
| C96 | GFTFSNYA | INSGGGNT | ATHNYESTVFDY | SSVSY | ETS | QQWNYPSMYT |
| | (SEQ ID NO: 23) | (SEQ ID | (SEQ ID NO: 3) | (SEQ ID | (SEQ ID NO: 5) | (SEQ ID NO: 6) |
| | | NO: 181) | | NO: 4) | | |
| C113 | GFTFSNYA | IHSGGGNT | VTHNYESTVFDY | SSVSY | ETS | QQWNYPSMYT |
| | (SEQ ID NO: 23) | (SEQ ID | (SEQ ID NO: 9) | (SEQ ID | (SEQ ID NO: 5) | (SEQ ID NO: 6) |
| | | NO: 182) | | NO: 4) | | |
| C134 | GFTFSNYG | ISNGGGNT | VTHNYESTVFDY | SSVSY | ETS | QQWNYPSMYT |
| | (SEQ ID NO: 7) | (SEQ ID | (SEQ ID NO: 9) | (SEQ ID | (SEQ ID NO: 5) | (SEQ ID NO: 6) |
| | | NO: 52) | | NO: 4) | | |
| C143 | GFTFSNYA | ISNGGGNS | ATHNYESTVFDY | SSVSY | ETS | QQWNYPSMYT |
| | (SEQ ID NO: 23) | (SEQ ID | (SEQ ID NO: 3) | (SEQ ID | (SEQ ID NO: 5) | (SEQ ID NO: 6) |
| | | NO: 183) | | NO: 4) | | |
| C148 | GFTFSNYG | IGSGGSDT | ATHNYESTVFDY | SSVSY | ETS | QQWNYPSMYT |
| | (SEQ ID NO: 7) | (SEQ ID | (SEQ ID NO: 3) | (SEQ ID | (SEQ ID NO: 5) | (SEQ ID NO: 6) |
| | | NO: 184) | | NO: 4) | | |
| C186 | GFTFSTYA | INSGGGDT | ATHNYESTVFDY | SSVSY | ETS | QQWNYPSMYT |
| | (SEQ ID | (SEQ ID | (SEQ ID NO: 3) | (SEQ ID | (SEQ ID NO: 5) | (SEQ ID NO: 6) |
| | NO: 185) | NO: 186) | | NO: 4) | | |
| C194 | GFTFSNYA | ISSGGGDT | ATHNYESTVFDY | SSVSY | ETS | QQWNYPSMYT |
| | (SEQ ID NO: 23) | (SEQ ID | (SEQ ID NO: 3) | (SEQ ID | (SEQ ID NO: 5) | (SEQ ID NO: 6) |
| | | NO: 34) | | NO: 4) | | |
| C216 | GFIFGNYA | VNSGGGNT | ATHNYESTVFDY | SSVSY | ETS | QQWNYPSMYT |
| | (SEQ ID | (SEQ ID | (SEQ ID NO: 3) | (SEQ ID | (SEQ ID NO: 5) | (SEQ ID NO: 6) |
| | NO: 187) | NO: 188) | | NO: 4) | | |

In one aspect, the CD160 binding domain may comprise the set of VH and VL sequences listed for one of the clones identified in Table 3 below.

The CD160 binding domain may comprise a VH region having the sequence shown as SEQ ID NO: 114 or a variant having at least 80%, 85%, 90%, 95%, 98% or 99% sequence identity thereto; and a VL region having the sequence shown as SEQ ID NO: 115 or a variant of having at least 80%, 85%, 90%, 95%, 98% or 99% sequence identity thereto. Suitably, the CD160 binding domain may comprise a VH region having the sequence shown as SEQ ID NO: 114; and a VL region having the sequence shown as SEQ ID NO: 115.

The CD160 binding domain may comprise a VH region having the sequence shown as SEQ ID NO: 116 or a variant having at least 80%, 85%, 90%, 95%, 98% or 99% sequence identity thereto; and a VL region having the sequence shown as SEQ ID NO: 117 or a variant of having at least 80%, 85%, 90%, 95%, 98% or 99% sequence identity thereto. Suitably, the CD160 binding domain may comprise a VH region having the sequence shown as SEQ ID NO: 116; and a VL region having the sequence shown as SEQ ID NO: 117.

The CD160 binding domain may comprise a VH region having the sequence shown as SEQ ID NO: 118 or a variant having at least 80%, 85%, 90%, 95%, 98% or 99% sequence identity thereto; and a VL region having the sequence shown as SEQ ID NO: 119 or a variant of having at least 80%, 85%, 90%, 95%, 98% or 99% sequence identity thereto. Suitably, the CD160 binding domain may comprise a VH region having the sequence shown as SEQ ID NO: 118; and a VL region having the sequence shown as SEQ ID NO: 119.

The CD160 binding domain may comprise a VH region having the sequence shown as SEQ ID NO: 120 or a variant having at least 80%, 85%, 90%, 95%, 98% or 99% sequence identity thereto; and a VL region having the sequence shown as SEQ ID NO: 121 or a variant of having at least 80%, 85%, 90%, 95%, 98% or 99% sequence identity thereto. Suitably, the CD160 binding domain may comprise a VH region having the sequence shown as SEQ ID NO: 120; and a VL region having the sequence shown as SEQ ID NO: 121.

The CD160 binding domain may comprise a VH region having the sequence shown as SEQ ID NO: 122 or a variant having at least 80%, 85%, 90%, 95%, 98% or 99% sequence identity thereto; and a VL region having the sequence shown as SEQ ID NO: 123 or a variant of having at least 80%, 85%, 90%, 95%, 98% or 99% sequence identity thereto. Suitably, the CD160 binding domain may comprise a VH region having the sequence shown as SEQ ID NO: 122; and a VL region having the sequence shown as SEQ ID NO: 123.

The CD160 binding domain may comprise a VH region having the sequence shown as SEQ ID NO: 124 or a variant having at least 80%, 85%, 90%, 95%, 98% or 99% sequence identity thereto; and a VL region having the sequence shown as SEQ ID NO: 125 or a variant of having at least 80%, 85%, 90%, 95%, 98% or 99% sequence identity thereto. Suitably, the CD160 binding domain may comprise a VH region having the sequence shown as SEQ ID NO: 124; and a VL region having the sequence shown as SEQ ID NO: 125.

The CD160 binding domain may comprise a VH region having the sequence shown as SEQ ID NO: 126 or a variant having at least 80%, 85%, 90%, 95%, 98% or 99% sequence identity thereto; and a VL region having the sequence shown as SEQ ID NO: 127 or a variant of having at least 80%, 85%, 90%, 95%, 98% or 99% sequence identity thereto. Suitably, the CD160 binding domain may comprise a VH region having the sequence shown as SEQ ID NO: 126; and a VL region having the sequence shown as SEQ ID NO: 127.

The CD160 binding domain may comprise a VH region having the sequence shown as SEQ ID NO: 128 or a variant having at least 80%, 85%, 90%, 95%, 98% or 99% sequence identity thereto; and a VL region having the sequence shown as SEQ ID NO: 129 or a variant of having at least 80%, 85%, 90%, 95%, 98% or 99% sequence identity thereto. Suitably, the CD160 binding domain may comprise a VH region having the sequence shown as SEQ ID NO: 128; and a VL region having the sequence shown as SEQ ID NO: 129.

The CD160 binding domain may comprise a VH region having the sequence shown as SEQ ID NO: 130 or a variant having at least 80%, 85%, 90%, 95%, 98% or 99% sequence identity thereto; and a VL region having the sequence shown as SEQ ID NO: 131 or a variant of having at least 80%, 85%, 90%, 95%, 98% or 99% sequence identity thereto. Suitably, the CD160 binding domain may comprise a VH region having the sequence shown as SEQ ID NO: 130; and a VL region having the sequence shown as SEQ ID NO: 131.

The CD160 binding domain may comprise a VH region having the sequence shown as SEQ ID NO: 132 or a variant having at least 80%, 85%, 90%, 95%, 98% or 99% sequence identity thereto; and a VL region having the sequence shown as SEQ ID NO: 133 or a variant of having at least 80%, 85%, 90%, 95%, 98% or 99% sequence identity thereto. Suitably, the CD160 binding domain may comprise a VH region having the sequence shown as SEQ ID NO: 132; and a VL region having the sequence shown as SEQ ID NO: 133.

The CD160 binding domain may comprise a VH region having the sequence shown as SEQ ID NO: 134 or a variant having at least 80%, 85%, 90%, 95%, 98% or 99% sequence identity thereto; and a VL region having the sequence shown as SEQ ID NO: 135 or a variant of having at least 80%, 85%, 90%, 95%, 98% or 99% sequence identity thereto. Suitably, the CD160 binding domain may comprise a VH region having the sequence shown as SEQ ID NO: 134; and a VL region having the sequence shown as SEQ ID NO: 135.

The CD160 binding domain may comprise a VH region having the sequence shown as SEQ ID NO: 136 or a variant having at least 80%, 85%, 90%, 95%, 98% or 99% sequence identity thereto; and a VL region having the sequence shown as SEQ ID NO: 137 or a variant of having at least 80%, 85%, 90%, 95%, 98% or 99% sequence identity thereto. Suitably, the CD160 binding domain may comprise a VH region having the sequence shown as SEQ ID NO: 136; and a VL region having the sequence shown as SEQ ID NO: 137.

TABLE 3

| Clone | VH sequence | VL sequence |
|---|---|---|
| 1 | EVKLVESGGGLVQPGRSLKLSCAASGFIFSNY | EIVLTQSPTTMTASPGEKVTITCRASSSVSYMHWYQ |
| | AMAWVRQAPTKGLEWVASVNSGGANTYYRD | QKPGASPKPWIYETSKLASGVPDRFSGSGSGTSYSL |
| | SVKGRFTISRDNAKNTQYLHIDSLRSEDTATYY | TINNMEAEDAATYYCQQWNYPSMYTFGAGTKLELK |
| | CATHNYESTVFDYWGQGAMVTVSS | (SEQ ID NO: 115) |
| | (SEQ ID NO: 114) | |
| 2 | EVQLVESGGDLVQPGRSLKLSCAASGFTFSNY | EIVLTQSPTTMTASPGEKVTITCRASSSVSYMHWYQ |
| | GMAWVRQAPTKGLEWVASVSNGGGNTYYRD | QKPGASPKPWIYETSKLASGVPDRFSGSGSGTSYSL |
| | SVKGRFTISRDNAKNTQYLQMDSLRSEDTATY | TINNMEAEDAATYYCQQWNYPSMYTFGPGTKLELK |
| | YCVTHNYESTVFDYWGQGAMVTVSS (SEQ ID | (SEQ ID NO: 117) |
| | NO: 116) | |
| 3 | QVNLQESGPGLVQPSETLSLTCTVSGFSLTSY | DIQMTQSPASLSASLGETVSIECLASEDIYNNLAWYQ |
| | GVYWVRQPPGKGLEWMGRIWNDGGTDYNPA | QKPGKSPQLLIYYASSLQDGVPSRFSGSGSGTQYSL |
| | LKSRLSISRDTSKSQVFLKLNSLQTEDTAIYFCA | KINSLESEDVATYFCLQDSEYPPTFGGGTKLELK |
| | RGWTHWGQGTLVTVSS (SEQ ID NO: 56) | (SEQ ID NO: 57) |
| 4 | EVKLVESGGGLVQPGRPLKLSCAASGFTFSNY | DIAITQSPASMTASPGERVTITCRASSSVSYMHWYQ |
| | GMAWVRQAPKKGLEWVATISTGDDNTYYRDS | QKPGASPKPWIYETSKLASGVPDRFSGSGSGTSYSL |
| | VKGRFTISRDNAKNTQYLQMDSLRSEDTATYY | TINNMEAEDAATYYCQQWNYPSMYTFGAGTKLELK |
| | CATHNYESTIFDYWGQGVMVTVSS (SEQ ID | (SEQ ID NO: 59) |
| | NO: 58) | |
| 5 | QVQLKESGPGLVQPSETLSLTCTVSGFSLTNY | DIQLTQSPSLLSASVGDRVTLSCKGSQNINNYLAWY |
| | GINWFRQPPGKGLEWMGTIWNDGGTDYNSAL | QQKLGEAPKLLIYNTNSLQTGIPSRFSGSGSGTDYTL |
| | KSRLTISRDTSKSQVFLKMNSLQTEDTAIYFCA | TISSLQPEDFATYFCLQHNSGYTFGAGTKLEIK |
| | RGMEAWGQGASVTVSS | (SEQ ID NO: 119) |
| | (SEQ ID NO: 118) | |
| 6 | EVQLVESGGGFVQPGRSLKLSCAASGFTFSN | EIILTQSPTTMTASPGEKVTITCRASSSVSYMHWYQQ |
| | YAMAWVRQAPTKGLEWVASINSGGGDSYYRD | KPGASPKPWIYETSKLASGVPDRFSGSGSGTSYSLT |
| | SVKGRFTISRDNAKNTQYLQMDSLRSEDTATY | INNMEAEDAATYYCQQWNYPSMYTFGAGTKLELK |
| | YCATHNYGSTIFDYWGQGAMVTVSS | (SEQ ID NO: 121) |
| | (SEQ ID NO: 120) | |
| 7 | EVQLVESGPGLVQPSETLSLTCTVSGFSLTSH | DIQMTQSPASLSASLGETVNIECLASEDIYSDLAWYQ |
| | GVNWFRQPPGKGLEWMGTIWNDGGTDYNSA | QKPGKSPQLLIYNANSLQNGVPSRFSGSGSGTQYSL |
| | LKSRLSIIRDTSKGQVFLKMNSLQTEDTAIYFCA | KINSLQSEDVATYFCQQYNNYPWTFGGGTKLEIK |
| | RGLAYWGQGTLVTVSS | (SEQ ID NO: 123) |
| | (SEQ ID NO: 122) | |
| 8 | QVNLQESGPGLVQPSETLSLTCTVSGFSLTNY | DIQMTQSPASLSASLGETVSIECLASEDIYNNLAWYQ |
| | GINWFRQPPGKGLEWMGTIWNDGGTDYNSAL | QKPGKSPQLLIYYANSLQDGVPSRFSGSGSGTQYSL |
| | KSRLSISRDTSKSQVFLKMNSLQTEDTAIYFCA | KINSLQSEDVATYFCQQYNNYPFTFGSGTKLELK |
| | RGMDAWGQGASVTVSS | (SEQ ID NO: 125) |
| | (SEQ ID NO: 124) | |
| 9 | EVQLVESGGGLVQPGRSLKLSCAASGFTFSNY | EIVLTQSPTTTAASPGERVTITCRASSSVSYMHWYQ |
| | AMAWVRQAPTKGLEWVASISSGGGDTYYRDS | QKPGASPKPWIYETSKLASGVPDRFSGSGSGTSYSL |
| | VKGRFTISRDNSKNTQYLQMDSLRSEDTATYY | TINNMEAEDAATYYCQQWNYPSMYTFGAGTKLEIK |
| | CATHNYESTVLITWGQGASVTVSS | (SEQ ID NO: 127) |
| | (SEQ ID NO: 126) | |
| 10 | EVQLVESGPGLVQPSETLSLTCTVSGFSLTSY | DIVMTQSPKSMSISVGDRVTMNCKASQNVGKNIAWY |
| | GVNWFRQPPGKGLEWMGTIWNDGGTDYNSA | QQKPGQSPKLLIYYASNRYTGVPDRFTGSGSGTDFT |
| | LKSRLSISRDTSKSQVFLKMNSLQTEDTAIYFC | LTITNMQTEDLAVYYCLQYNYNPWTFGGGTKLELK |
| | ARGFEYWGQGTLVTVSS | (SEQ ID NO: 129) |
| | (SEQ ID NO: 128) | |
| 11 | EVQLVESGGGLVQPGRSLKLSCAASGFTFSNY | DIVLTQSPALAVSPGERVSISCRASESVSTLMHWYQ |
| | GMAWVRQAPTKGLEWVASINTSGGSTYYRDS | QKLGQQPKLLIYGASNLESGVPARFSGSGSGTDFTL |
| | VKGRFTISRDNAKSTLYLQMDSLRSEDTATYY | NIDPVEADDIATYFCQQSWNGPWTFGGGTKLELK |
| | CATIYYHDGSYYYAGWFAYWGQGTLVTVSS | (SEQ ID NO: 131) |
| | (SEQ ID NO: 130) | |
| 12 | EVQLVESGGGFVQPGGSLKLSCAASGFTFSN | EIVLTQSPTTMTASPGEKVTIICRASSSVSYMHWYQQ |
| | YGMAWVRQAPKKGLEWVASISSGRGNTYYRD | KPGASPKPWIYETSKLASGVPDRFSGSGSGTSYSLT |
| | SVKGRFTISRDNAKNTQYLQMDSLRSEDTATY | FNNMEAEDAATYYCQQWNYPSMYTFGAGTKLELK |
| | YCVTHNYESTIFDYWGQGVMVTVSS (SEQ ID | (SEQ ID NO: 61) |
| | NO: 60) | |
| 16 | QVNLQESGPGLVRPSETLSLTCTVSGFSLISYG | DIQMTQSPASLSASLGETVSIECLTSEDISNDSAWYQ |
| | VNWFRQPPGKGLEWMGTIWNDGGTDYNSAL | QKSGKSPQLLIYAASRLQDGVPSRFSGSGSGTRYSL |
| | KSRLSISRDTSKSQVFLKMNSLQTEDTAIYFCA | KISGMQPEDEADYFCQQSYKYPLTFGSGTKLEIK |
| | RGFEHWGQGVMVTVSS | (SEQ ID NO: 133) |
| | (SEQ ID NO: 132) | |
| 19 | EVKLVESGGGLVQPGRSLKLSCAASGFTFSNY | EIVLTQSPTPMTASPGEKVTITCRASSSVSYMHWYQ |
| | GMAWVRQAPTKGLEWVATISNGGGNTYYRDS | QKPGASPKPWIYETSKLASGVPDRFSGSGSGTSYSL |
| | VKGRFTISRDNAKNTQYLQMDSLRSEDTATYY | TINNMEAEDAATYYCQQWNYPSMYTFGAGTKLELK |
| | CVAHNYESTVFDYWGQGAMVTVSS | (SEQ ID NO: 135) |
| | (SEQ ID NO: 134) | |
| 20 | EVKLVESGGGLVQPGRSLKLSCTASGFTFSNY | EIVLTQSPTTTTASPGERVTITCRASSSVSYMHWYQ |
| | ATAWVRQAPTKGLEWVASIITGVGNTYYRDSV | QKPGASPKPWIYETSKLASGVPDRFSGSGSGTSYSL |
| | KGRFTISRDNARNTQYLQMDSLRSEDTATYYC | TINNMEAEDAATYYCQQWNYPSMYTFGAGTKLEIK |
| | EHITTSPPFLITWGQGSWSQSPQ | (SEQ ID NO: 137) |
| | (SEQ ID NO: 136) | |

In one aspect, the CD160 binding domain may comprise the set of VH and VL sequences listed for one of the clones identified in Table 4 below.

The CD160 binding domain may comprise a VH region having the sequence shown as SEQ ID NO: 138 or a variant having at least 80%, 85%, 90%, 95%, 98% or 99% sequence identity thereto; and a VL region having the sequence shown as SEQ ID NO: 139 or a variant of having at least 80%, 85%, 90%, 95%, 98% or 99% sequence identity thereto. Suitably, the CD160 binding domain may comprise a VH region having the sequence shown as SEQ ID NO: 138; and a VL region having the sequence shown as SEQ ID NO: 139.

The CD160 binding domain may comprise a VH region having the sequence shown as SEQ ID NO: 140 or a variant having at least 80%, 85%, 90%, 95%, 98% or 99% sequence identity thereto; and a VL region having the sequence shown as SEQ ID NO: 141 or a variant of having at least 80%, 85%, 90%, 95%, 98% or 99% sequence identity thereto. Suitably, the CD160 binding domain may comprise a VH region having the sequence shown as SEQ ID NO: 140; and a VL region having the sequence shown as SEQ ID NO: 141.

The CD160 binding domain may comprise a VH region having the sequence shown as SEQ ID NO: 142 or a variant having at least 80%, 85%, 90%, 95%, 98% or 99% sequence identity thereto; and a VL region having the sequence shown as SEQ ID NO: 143 or a variant of having at least 80%, 85%, 90%, 95%, 98% or 99% sequence identity thereto. Suitably, the CD160 binding domain may comprise a VH region having the sequence shown as SEQ ID NO: 142; and a VL region having the sequence shown as SEQ ID NO: 143.

The CD160 binding domain may comprise a VH region having the sequence shown as SEQ ID NO: 144 or a variant having at least 80%, 85%, 90%, 95%, 98% or 99% sequence identity thereto; and a VL region having the sequence shown as SEQ ID NO: 145 or a variant of having at least 80%, 85%, 90%, 95%, 98% or 99% sequence identity thereto. Suitably, the CD160 binding domain may comprise a VH region having the sequence shown as SEQ ID NO: 144; and a VL region having the sequence shown as SEQ ID NO: 145.

The CD160 binding domain may comprise a VH region having the sequence shown as SEQ ID NO: 146 or a variant having at least 80%, 85%, 90%, 95%, 98% or 99% sequence identity thereto; and a VL region having the sequence shown as SEQ ID NO: 135 or a variant of having at least 80%, 85%, 90%, 95%, 98% or 99% sequence identity thereto. Suitably, the CD160 binding domain may comprise a VH region having the sequence shown as SEQ ID NO: 146; and a VL region having the sequence shown as SEQ ID NO: 135.

The CD160 binding domain may comprise a VH region having the sequence shown as SEQ ID NO: 148 or a variant having at least 80%, 85%, 90%, 95%, 98% or 99% sequence identity thereto; and a VL region having the sequence shown as SEQ ID NO: 149 or a variant of having at least 80%, 85%, 90%, 95%, 98% or 99% sequence identity thereto. Suitably, the CD160 binding domain may comprise a VH region having the sequence shown as SEQ ID NO: 148; and a VL region having the sequence shown as SEQ ID NO: 149.

The CD160 binding domain may comprise a VH region having the sequence shown as SEQ ID NO: 150 or a variant having at least 80%, 85%, 90%, 95%, 98% or 99% sequence identity thereto; and a VL region having the sequence shown as SEQ ID NO: 151 or a variant of having at least 80%, 85%, 90%, 95%, 98% or 99% sequence identity thereto. Suitably, the CD160 binding domain may comprise a VH region having the sequence shown as SEQ ID NO: 150; and a VL region having the sequence shown as SEQ ID NO: 151.

The CD160 binding domain may comprise a VH region having the sequence shown as SEQ ID NO: 152 or a variant having at least 80%, 85%, 90%, 95%, 98% or 99% sequence identity thereto; and a VL region having the sequence shown as SEQ ID NO: 153 or a variant of having at least 80%, 85%, 90%, 95%, 98% or 99% sequence identity thereto. Suitably, the CD160 binding domain may comprise a VH region having the sequence shown as SEQ ID NO: 152; and a VL region having the sequence shown as SEQ ID NO: 153.

The CD160 binding domain may comprise a VH region having the sequence shown as SEQ ID NO: 154 or a variant having at least 80%, 85%, 90%, 95%, 98% or 99% sequence identity thereto; and a VL region having the sequence shown as SEQ ID NO: 127 or a variant of having at least 80%, 85%, 90%, 95%, 98% or 99% sequence identity thereto. Suitably, the CD160 binding domain may comprise a VH region having the sequence shown as SEQ ID NO: 154; and a VL region having the sequence shown as SEQ ID NO: 127.

The CD160 binding domain may comprise a VH region having the sequence shown as SEQ ID NO: 156 or a variant having at least 80%, 85%, 90%, 95%, 98% or 99% sequence identity thereto; and a VL region having the sequence shown as SEQ ID NO: 121 or a variant of having at least 80%, 85%, 90%, 95%, 98% or 99% sequence identity thereto. Suitably, the CD160 binding domain may comprise a VH region having the sequence shown as SEQ ID NO: 156; and a VL region having the sequence shown as SEQ ID NO: 121.

TABLE 4

| Clone | VH sequence | VL sequence |
|---|---|---|
| C68 | EVQLVESGGGLVQPGRSLKLSCAASRFTFSNY | ETVLTQSPTTTPASPGEKVTITCRASSSVSYMHWYQQ |
| | AMAWVRQAPTKGLEWVASISSGAGNTYYRDS | KSGASPKLWIYDTSKLASGVPNRFSGSGSGTSYSLTIN |
| | VKGRFTISRDNAKNTQYLQMGSLRSEDTATYY | NMEAEDAATYYCQQWNYPSMYTFGPGTKLEIK |
| | CVTHNYESTVFDYWGQGASVTVSS | (SEQ ID NO: 139) |
| | (SEQ ID NO: 138) | |
| C92 | QVNLQESGPGLVQPSETLSLTCTVSGFSLTNY | DIRMTQSPASLSASLGETVSIECLASEDIYKNLAWYQQ |
| | GINWFRQPPGKGLEWMGTIWNDGGTDYNSAL | KPGKSPQLLIYYASSLQDGVPSRFSGSGSGTQYSLKIN |
| | KSRLTISRDTSKSQVFLKMNSLQTEDTAIYFCA | SLESEDAATYFCLQDSEYPLTFGSGTKLEIK |
| | RGMEAWGQGASVTVSS | (SEQ ID NO: 141) |
| | (SEQ ID NO: 140) | |
| C96 | EVQLVESGGGLVQPGRSLKLSCAASGFTFSNY | EIVLTQSPTSMTASPGERVTITCRASSSVSYMHWYQQ |
| | AMAWVRQAPTKGLEWVASINSGGGNTYYRDS | KPGASPKPWIYETSKLASGVPDRFSGSGSGTSYSLTIN |
| | VKGRFTISRDNAKNTQYLQMDSLRSEDTATYY | NMEAEDAATYYCQQWNYPSMYTFGAGTKLEIK |
| | CATHNYESTVFDYWGQGVMVTVSS | (SEQ ID NO: 143) |
| | (SEQ ID NO: 142) | |
| C113 | EVKLVESGGGLVQPGRSLKLSCAASGFTFSNY | EIVLTQSPTTMTASPGEKVTITCRASSSVSYMHWYQQ |
| | AMAWVRQAPTKGLEWVASIHSGGGNTYYRDS | KPGASPKPWIYETSKLASGVPDRFSGSGSGTSYSLTIN |
| | VKGRFTISRDNAKNTQYLQMDSLRSEDTATYY | NMEAEDAATYYCQQWNYPSMYTFGAGTKLEIK |
| | CVTHNYESTVFDYWGQGAMVTVSS | (SEQ ID NO: 145) |
| | (SEQ ID NO: 144) | |
| C134 | EVKLVESGGGLVQPGRSLKLSCAASGFTFSNY | EIVLTQSPTPMTASPGEKVTITCRASSSVSYMHWYQQ |
| | GMAWVRQAPTKGLEWVATISNGGGNTYYRDS | KPGASPKPWIYETSKLASGVPDRFSGSGSGTSYSLTIN |
| | VKGRFTISRDNAKNTQYLQMDSLRSEDTATYY | NMEAEDAATYYCQQWNYPSMYTFGAGTKLELK |
| | CVTHNYESTVFDYWGQGAMVTVSS | (SEQ ID NO: 135) |
| | (SEQ ID NO: 146) | |
| C143 | EVQLVESGGDLVQPGRSLKLSCAASGFTFSNY | EIVLTQSPTTMTASPGEKVTITCRASSSVSYMHWYQQ |
| | AMAWVRQAPTKGLEWVASISNGGGNSFYRDS | KPGASPKPWIYETSKLASGVPDRFSGSGSGTSYSLTIN |
| | VKGRFTISRDNAKNTQYLQMDSLRSEDTATYY | NMEAEDAATYYCQQWNYPSMYTFGPGTKLEIK |
| | CATHNYESTVFDYWGRGVMVTVSS | (SEQ ID NO: 149) |
| | (SEQ ID NO: 148) | |
| C148 | EVQLVESGGGLVQPGRSLKLSCAASGFTFSNY | DTVLTQSPTSMTASPGERVTITCRASSSVSYMHWYQQ |
| | GMAWVRQAPTKGLEWVASIGSGGSDTYYRDS | KPGASPKPWIYETSKLASGVPDRFSGSGSGTSYSLTIN |
| | VKGRFTISRDNAKNTQYLQMDSLRSEDTATYY | NMEAEDAATYYCQQWNYPSMYTFGAGTKLEIK |
| | CATHNYESTVFDYWGQGVMVTVSS | (SEQ ID NO: 151) |
| | (SEQ ID NO: 150) | |
| C186 | EVKLVESGGGLVQPGRSLKLSCAASGFTFSTY | EIVLTQSPTTMTASPGEKVTITCRASSSVSYMHWYQQ |
| | AMAWVRQAPTKGLEWVASINSGGGDTYYRDS | KPGASPKPWIYETSKLASGVPDRFSGSGSGTSYSLTF |
| | VKGRFTISRDNTKNTQYLQMDSLRSEDTATYY | NNMEAEDAATYYCQQWNYPSMYTFGAGTKLELK |
| | CATHNYESTVFDYWGQGVMVTVSS | (SEQ ID NO: 153) |
| | (SEQ ID NO: 152) | |
| C194 | EVQLVESGGGLVQPGRSLKLSCAASGFTFSNY | EIVLTQSPTTTAASPGERVTITCRASSSVSYMHWYQQK |
| | AMAWVRQAPTKGLEWVASISSGGGDTYYRDS | PGASPKPWIYETSKLASGVPDRFSGSGSGTSYSLTINN |
| | VKGRFTISRDNSKNTQYLQMDSLRSEDTATYY | MEAEDAATYYCQQWNYPSMYTFGAGTKLEIK |
| | CATHNYESTVFDYWGQGASVTVSS | (SEQ ID NO: 127) |
| | (SEQ ID NO: 154) | |
| C216 | EVQLVESGGGLVQPGRSLKLSCAASGFIFGNY | EIILTQSPTTMTASPGEKVTITCRASSSVSYMHWYQQK |
| | AMAWVRQAPTKGLEWVASVNSGGGNTYYRD | PGASPKPWIYETSKLASGVPDRFSGSGSGTSYSLTINN |
| | SVKGRFTISRDNAKNTQYLHIDGLRSEDTATYY | MEAEDAATYYCQQWNYPSMYTFGAGTKLELK |
| | CATHNYESTVFDYWGQGASVTVSS | (SEQ ID NO: 121) |
| | (SEQ ID NO: 156) | |

In one aspect, the CD160 binding domain may comprise the scFv sequences listed for one of the clones identified in Table 5 below.

Suitably, the CD160 binding domain comprises the amino acid sequence shown as SEQ ID NO: 158 or a variant thereof at least 80%, 85%, 90%, 95%, 98% or 99% sequence identity thereto.

Suitably, the CD160 binding domain comprises the amino acid sequence shown as SEQ ID NO: 159 or a variant thereof at least 80%, 85%, 90%, 95%, 98% or 99% sequence identity thereto.

Suitably, the CD160 binding domain comprises the amino acid sequence shown as SEQ ID NO: 160 or a variant thereof at least 80%, 85%, 90%, 95%, 98% or 99% sequence identity thereto.

Suitably, the CD160 binding domain comprises the amino acid sequence shown as SEQ ID NO: 161 or a variant thereof at least 80%, 85%, 90%, 95%, 98% or 99% sequence identity thereto.

Suitably, the CD160 binding domain comprises the amino acid sequence shown as SEQ ID NO: 162 or a variant thereof at least 80%, 85%, 90%, 95%, 98% or 99% sequence identity thereto.

Suitably, the CD160 binding domain comprises the amino acid sequence shown as SEQ ID NO: 163 or a variant thereof at least 80%, 85%, 90%, 95%, 98% or 99% sequence identity thereto.

Suitably, the CD160 binding domain comprises the amino acid sequence shown as SEQ ID NO: 164 or a variant thereof at least 80%, 85%, 90%, 95%, 98% or 99% sequence identity thereto.

Suitably, the CD160 binding domain comprises the amino acid sequence shown as SEQ ID NO: 165 or a variant thereof at least 80%, 85%, 90%, 95%, 98% or 99% sequence identity thereto.

Suitably, the CD160 binding domain comprises the amino acid sequence shown as SEQ ID NO: 166 or a variant thereof at least 80%, 85%, 90%, 95%, 98% or 99% sequence identity thereto.

Suitably, the CD160 binding domain comprises the amino acid sequence shown as SEQ ID NO: 167 or a variant thereof at least 80%, 85%, 90%, 95%, 98% or 99% sequence identity thereto.

Suitably, the CD160 binding domain comprises the amino acid sequence shown as SEQ ID NO: 168 or a variant thereof at least 80%, 85%, 90%, 95%, 98% or 99% sequence identity thereto.

Suitably, the CD160 binding domain comprises the amino acid sequence shown as SEQ ID NO: 169 or a variant thereof at least 80%, 85%, 90%, 95%, 98% or 99% sequence identity thereto.

TABLE 5

| Clone | scFv sequence |
|---|---|
| 1 | EVKLVESGGGLVQPGRSLKLSCAASGFIFSNYAMAWVRQAPTKGLEWVASVNSGGANTYYRDSVKG |
| | RFTISRDNAKNTQYLHIDSLRSEDTATYYCATHNYESTVFDYWGQGAMVTVSSGGGGSGGGGSSGG |
| | GGSEIVLTQSPTTMTASPGEKVTITCRASSSVSYMHWYQQKPGASPKPWIYETSKLASGVPDRFSGS |
| | GSGTSYSLTINNMEAEDAATYYCQQWNYPSMYTFGAGTKLELKR |
| | (SEQ ID NO: 158) |
| 2 | EVQLVESGGDLVQPGRSLKLSCAASGFTFSNYGMAWVRQAPTKGLEWVASVSNGGGNTYYRDSVK |
| | GRFTISRDNAKNTQYLQMDSLRSEDTATYYCVTHNYESTVFDYWGQGAMVTVSSGGGGSGGGGSG |
| | GGGSEIVLTQSPTTMTASPGEKVTITCRASSSVSYMHWYQQKPGASPKPWIYETSKLASGVPDRFSG |
| | SGSGTSYSLTINNMEAEDAATYYCQQWNYPSMYTFGPGTKLELKR |
| | (SEQ ID NO: 159) |
| 3 | QVNLQESGPGLVQPSETLSLTCTVSGFSLTSYGVYWVRQPPGKGLEWMGRIWNDGGTDYNPALKSR |
| | LSISRDTSKSQVFLKLNSLQTEDTAIYFCARGWTHWGQGTLVTVSSGGGGSGGGGSGGGGSDIQMT |
| | QSPASLSASLGETVSIECLASEDIYNNLAWYQQKPGKSPQLLIYYASSLQDGVPSRFSGSGSGTQYSL |
| | KINSLESEDVATYFCLQDSEYPPTFGGGTKLELKR (SEQ ID NO: 62) |
| 4 | EVKLVESGGGLVQPGRPLKLSCAASGFTFSNYGMAWVRQAPKKGLEWVATISTGDDNTYYRDSVKG |
| | RFTISRDNAKNTQYLQMDSLRSEDTATYYCATHNYESTIFDYWGQGVMVTVSSGGGGSGGGGSGGG |
| | GSDIAITQSPASMTASPGERVTITCRASSSVSYMHWYQQKPGASPKPWIYETSKLASGVPDRFSGSG |
| | SGTSYSLTINNMEAEDAATYYCQQWNYPSMYTFGAGTKLELKR (SEQ ID NO: 63) |
| 5 | QVQLKESGPGLVQPSETLSLTCTVSGFSLTNYGINWFRQPPGKGLEWMGTIWNDGGTDYNSALKSRL |
| | TISRDTSKSQVFLKMNSLQTEDTAIYFCARGMEAWGQGASVTVSSGGGGSGGGGSGGGGSDIQLTQ |
| | SPSLLSASVGDRVTLSCKGSQNINNYLAWYQQKLGEAPKLLIYNTNSLQTGIPSRFSGSGSGTDYTLTI |
| | SSLQPEDFATYFCLQHNSGYTFGAGTKLEIKR |
| | (SEQ ID NO: 160) |
| 6 | EVQLVESGGGFVQPGRSLKLSCAASGFTFSNYAMAWVRQAPTKGLEWVASINSGGGDSYYRDSVKG |
| | RFTISRDNAKNTQYLQMDSLRSEDTATYYCATHNYGSTIFDYWGQGAMVTVSSGGGGSGGGGSGG |
| | GGSEIILTQSPTTMTASPGEKVTITCRASSSVSYMHWYQQKPGASPKPWIYETSKLASGVPDRFSGSG |
| | SGTSYSLTINNMEAEDAATYYCQQWNYPSMYTFGAGTKLELKR |
| | (SEQ ID NO: 161) |
| 7 | EVQLVESGPGLVQPSETLSLTCTVSGFSLTSHGVNWFRQPPGKGLEWMGTIWNDGGTDYNSALKSR |
| | LSIIRDTSKGQVFLKMNSLQTEDTAIYFCARGLAYWGQGTLVTVSSGGGGSGGGGSGGGGSDIQMTQ |
| | SPASLSASLGETVNIECLASEDIYSDLAWYQQKPGKSPQLLIYNANSLQNGVPSRFSGSGSGTQYSLKI |
| | NSLQSEDVATYFCQQYNNYPWTFGGGTKLEIKR |
| | (SEQ ID NO: 162) |
| 8 | QVNLQESGPGLVQPSETLSLTCTVSGFSLTNYGINWFRQPPGKGLEWMGTIWNDGGTDYNSALKSRL |
| | SISRDTSKSQVFLKMNSLQTEDTAIYFCARGMDAWGQGASVTVSSGGGGSGGGGSGGGGSDIQMT |
| | QSPASLSASLGETVSIECLASEDIYNNLAWYQQKPGKSPQLLIYYANSLQDGVPSRFSGSGSGTQYSL |
| | KINSLQSEDVATYFCQQYNNYPFTFGSGTKLELKR |
| | (SEQ ID NO: 163) |
| 9 | EVQLVESGGGLVQPGRSLKLSCAASGFTFSNYAMAWVRQAPTKGLEWVASISSGGGDTYYRDSVKG |
| | RFTISRDNSKNTQYLQMDSLRSEDTATYYCATHNYESTVLITWGQGASVTVSSGGGGSGGGGSGGG |
| | GSEIVLTQSPTTTAASPGERVTITCRASSSVSYMHWYQQKPGASPKPWIYETSKLASGVPDRFSGSGS |
| | GTSYSLTINNMEAEDAATYYCQQWNYPSMYTFGAGTKLEIKR |
| | (SEQ ID NO: 164) |
| 10 | EVQLVESGPGLVQPSETLSLTCTVSGFSLTSYGVNWFRQPPGKGLEWMGTIWNDGGTDYNSALKSR |
| | LSISRDTSKSQVFLKMNSLQTEDTAIYFCARGFEYWGQGTLVTVSSGGGGSGGGGSGGGGSDIVMT |
| | QSPKSMSISVGDRVTMNCKASQNVGKNIAWYQQKPGQSPKLLIYYASNRYTGVPDRFTGSGSGTDFT |
| | LTITNMQTEDLAVYYCLQYNYNPWTFGGGTKLELKR |
| | (SEQ ID NO: 165) |
| 11 | EVQLVESGGGLVQPGRSLKLSCAASGFTFSNYGMAWVRQAPTKGLEWVASINTSGGSTYYRDSVKG |
| | RFTISRDNAKSTLYLQMDSLRSEDTATYYCATIYYHDGSYYYAGWFAYWGQGTLVTVSSGGGGSGG |
| | GGSGGGGSDIVLTQSPALAVSPGERVSISCRASESVSTLMHWYQQKLGQQPKLLIYGASNLESGVPA |
| | RFSGSGSGTDFTLNIDPVEADDIATYFCQQSWNGPWTFGGGTKLELKR |
| | (SEQ ID NO: 166) |
| 12 | EVQLVESGGGFVQPGGSLKLSCAASGFTFSNYGMAWVRQAPKKGLEWVASISSGRGNTYYRDSVKG |
| | RFTISRDNAKNTQYLQMDSLRSEDTATYYCVTHNYESTIFDYWGQGVMVTVSSGGGGSGGGGSGGG |
| | GSEIVLTQSPTTMTASPGEKVTIICRASSSVSYMHWYQQKPGASPKPWIYETSKLASGVPDRFSGSGS |
| | GTSYSLTFNNMEAEDAATYYCQQWNYPSMYTFGAGTKLELKR (SEQ ID NO: 64) |
| 16 | QVNLQESGPGLVRPSETLSLTCTVSGFSLISYGVNWFRQPPGKGLEWMGTIWNDGGTDYNSALKSRL |
| | SISRDTSKSQVFLKMNSLQTEDTAIYFCARGFEHWGQGVMVTVSSGGGGSGGGGSGGGGSDIQMT |
| | QSPASLSASLGETVSIECLTSEDISNDSAWYQQKSGKSPQLLIYAASRLQDGVPSRFSGSGSGTRYSL |
| | KISGMQPEDEADYFCQQSYKYPLTFGSGTKLEIKR |
| | (SEQ ID NO: 167) |
| 19 | EVKLVESGGGLVQPGRSLKLSCAASGFTFSNYGMAWVRQAPTKGLEWVATISNGGGNTYYRDSVKG |
| | RFTISRDNAKNTQYLQMDSLRSEDTATYYCVAHNYESTVFDYWGQGAMVTVSSGGGGSGGGGSGG |
| | GGSEIVLTQSPTPMTASPGEKVTITCRASSSVSYMHWYQQKPGASPKPWIYETSKLASGVPDRFSGS |
| | GSGTSYSLTINNMEAEDAATYYCQQWNYPSMYTFGAGTKLELKR |
| | (SEQ ID NO: 168) |
| 20 | EVKLVESGGGLVQPGRSLKLSCTASGFTFSNYATAWVRQAPTKGLEWVASIITGVGNTYYRDSVKGR |
| | FTISRDNARNTQYLQMDSLRSEDTATYYCEHITTSPPFLITWGQGSWSQSPQGGGGSGGGGGGGG |
| | SEIVLTQSPTTTTASPGERVTITCRASSSVSYMHWYQQKPGASPKPWIYETSKLASGVPDRFSGSGSG |
| | TSYSLTINNMEAEDAATYYCQQWNYPSMYTFGAGTKLEIKR |
| | (SEQ ID NO: 169) |

In one aspect, the CD160 binding domain may comprise the scFv sequences listed for one of the clones identified in Table 6 below.

Suitably, the CD160 binding domain comprises the amino acid sequence shown as SEQ ID NO: 170 or a variant thereof at least 80%, 85%, 90%, 95%, 98% or 99% sequence identity thereto.

Suitably, the CD160 binding domain comprises the amino acid sequence shown as SEQ ID NO: 171 or a variant thereof at least 80%, 85%, 90%, 95%, 98% or 99% sequence identity thereto.

Suitably, the CD160 binding domain comprises the amino acid sequence shown as SEQ ID NO: 172 or a variant thereof at least 80%, 85%, 90%, 95%, 98% or 99% sequence identity thereto.

Suitably, the CD160 binding domain comprises the amino acid sequence shown as SEQ ID NO: 173 or a variant thereof at least 80%, 85%, 90%, 95%, 98% or 99% sequence identity thereto.

Suitably, the CD160 binding domain comprises the amino acid sequence shown as SEQ ID NO: 174 or a variant thereof at least 80%, 85%, 90%, 95%, 98% or 99% sequence identity thereto.

Suitably, the CD160 binding domain comprises the amino acid sequence shown as SEQ ID NO: 175 or a variant thereof at least 80%, 85%, 90%, 95%, 98% or 99% sequence identity thereto.

Suitably, the CD160 binding domain comprises the amino acid sequence shown as SEQ ID NO: 176 or a variant thereof at least 80%, 85%, 90%, 95%, 98% or 99% sequence identity thereto.

Suitably, the CD160 binding domain comprises the amino acid sequence shown as SEQ ID NO: 177 or a variant thereof at least 80%, 85%, 90%, 95%, 98% or 99% sequence identity thereto.

Suitably, the CD160 binding domain comprises the amino acid sequence shown as SEQ ID NO: 178 or a variant thereof at least 80%, 85%, 90%, 95%, 98% or 99% sequence identity thereto.

Suitably, the CD160 binding domain comprises the amino acid sequence shown as SEQ ID NO: 179 or a variant thereof at least 80%, 85%, 90%, 95%, 98% or 99% sequence identity thereto.

TABLE 6

| Clone | scFv sequence |
|---|---|
| C68 | EVQLVESGGGLVQPGRSLKLSCAASRFTFSNYAMAWVRQAPTKGLEWVASISSGAGNTYYRDSVKGR |
| | FTISRDNAKNTQYLQMGSLRSEDTATYYCVTHNYESTVFDYWGQGASVTVSSGGGGSGGGGSGGGG |
| | SETVLTQSPTTTPASPGEKVTITCRASSSVSYMHWYQQKSGASPKLWIYDTSKLASGVPNRFSGSGSGT |
| | SYSLTINNMEAEDAATYYCQQWNYPSMYTFGPGTKLEIKR |
| | (SEQ ID NO: 170) |
| C92 | QVNLQESGPGLVQPSETLSLTCTVSGFSLTNYGINWFRQPPGKGLEWMGTIWNDGGTDYNSALKSRLTI |
| | SRDTSKSQVFLKMNSLQTEDTAIYFCARGMEAWGQGASVTVSSGGGGSGGGGSGGGGSDIRMTQSP |
| | ASLSASLGETVSIECLASEDIYKNLAWYQQKPGKSPQLLIYYASSLQDGVPSRFSGSGSGTQYSLKINSLE |
| | SEDAATYFCLQDSEYPLTFGSGTKLEIKR |
| | (SEQ ID NO: 171) |
| C96 | EVQLVESGGGLVQPGRSLKLSCAASGFTFSNYAMAWVRQAPTKGLEWVASINSGGGNTYYRDSVKGR |
| | FTISRDNAKNTQYLQMDSLRSEDTATYYCATHNYESTVFDYWGQGVMVTVSSGGGGSGGGGSGGGG |
| | SEIVLTQSPTSMTASPGERVTITCRASSSVSYMHWYQQKPGASPKPWIYETSKLASGVPDRFSGSGSGT |
| | SYSLTINNMEAEDAATYYCQQWNYPSMYTFGAGTKLEIKR |
| | (SEQ ID NO: 172) |
| C113 | EVKLVESGGGLVQPGRSLKLSCAASGFTFSNYAMAWVRQAPTKGLEWVASIHSGGGNTYYRDSVKGR |
| | FTISRDNAKNTQYLQMDSLRSEDTATYYCVTHNYESTVFDYWGQGAMVTVSSGGGGSGGGGSGGGG |
| | SEIVLTQSPTTMTASPGEKVTITCRASSSVSYMHWYQQKPGASPKPWIYETSKLASGVPDRFSGSGSGT |
| | SYSLTINNMEAEDAATYYCQQWNYPSMYTFGAGTKLEIKR |
| | (SEQ ID NO: 173) |
| C134 | EVKLVESGGGLVQPGRSLKLSCAASGFTFSNYGMAWVRQAPTKGLEWVATISNGGGNTYYRDSVKGR |
| | FTISRDNAKNTQYLQMDSLRSEDTATYYCVTHNYESTVFDYWGQGAMVTVSSGGGGSGGGGSGGGG |
| | SEIVLTQSPTPMTASPGEKVTITCRASSSVSYMHWYQQKPGASPKPWIYETSKLASGVPDRFSGSGSGT |
| | SYSLTINNMEAEDAATYYCQQWNYPSMYTFGAGTKLELKR |
| | (SEQ ID NO: 174) |
| C143 | EVQLVESGGDLVQPGRSLKLSCAASGFTFSNYAMAWVRQAPTKGLEWVASISNGGGNSFYRDSVKGR |
| | FTISRDNAKNTQYLQMDSLRSEDTATYYCATHNYESTVFDYWGRGVMVTVSSGGGGSGGGGSGGGG |
| | SEIVLTQSPTTMTASPGEKVTITCRASSSVSYMHWYQQKPGASPKPWIYETSKLASGVPDRFSGSGSGT |
| | SYSLTINNMEAEDAATYYCQQWNYPSMYTFGPGTKLEIKR |
| | (SEQ ID NO: 175) |
| C148 | EVQLVESGGGLVQPGRSLKLSCAASGFTFSNYGMAWVRQAPTKGLEWVASIGSGGSDTYYRDSVKGR |
| | FTISRDNAKNTQYLQMDSLRSEDTATYYCATHNYESTVFDYWGQGVMVTVSSGGGGSGGGGSGGGG |
| | SDTVLTQSPTSMTASPGERVTITCRASSSVSYMHWYQQKPGASPKPWIYETSKLASGVPDRFSGSGSG |
| | TSYSLTINNMEAEDAATYYCQQWNYPSMYTFGAGTKLEIKR |
| | (SEQ ID NO: 176) |
| C186 | EVKLVESGGGLVQPGRSLKLSCAASGFTFSTYAMAWVRQAPTKGLEWVASINSGGGDTYYRDSVKGR |
| | FTISRDNTKNTQYLQMDSLRSEDTATYYCATHNYESTVFDYWGQGVMVTVSSGGGGSGGGGSGGGG |
| | SEIVLTQSPTTMTASPGEKVTITCRASSSVSYMHWYQQKPGASPKPWIYETSKLASGVPDRFSGSGSGT |
| | SYSLTFNNMEAEDAATYYCQQWNYPSMYTFGAGTKLELKR |
| | (SEQ ID NO: 177) |
| C194 | EVQLVESGGGLVQPGRSLKLSCAASGFTFSNYAMAWVRQAPTKGLEWVASISSGGGDTYYRDSVKGR |
| | FTISRDNSKNTQYLQMDSLRSEDTATYYCATHNYESTVFDYWGQGASVTVSSGGGGSGGGGSGGGG |
| | SEIVLTQSPTTTAASPGERVTITCRASSSVSYMHWYQQKPGASPKPWIYETSKLASGVPDRFSGSGSGT |
| | SYSLTINNMEAEDAATYYCQQWNYPSMYTFGAGTKLEIKR |
| | (SEQ ID NO: 178) |
| C216 | EVQLVESGGGLVQPGRSLKLSCAASGFIFGNYAMAWVRQAPTKGLEWVASVNSGGGNTYYRDSVKGR |
| | FTISRDNAKNTQYLHIDGLRSEDTATYYCATHNYESTVFDYWGQGASVTVSSGGGGSGGGGSGGGGS |
| | EIILTQSPTTMTASPGEKVTITCRASSSVSYMHWYQQKPGASPKPWIYETSKLASGVPDRFSGSGSGTS |
| | YSLTINNMEAEDAATYYCQQWNYPSMYTFGAGTKLELKR |
| | (SEQ ID NO: 179) |

The term “polypeptide” is used in the conventional sense to mean a series of amino acids, typically L-amino acids, connected one to the other, typically by peptide bonds between the α-amino and carboxyl groups of adjacent amino acids. The term “polypeptide” is used interchangeably with the terms “amino acid sequence”, “peptide” and/or “protein”. The term “residues” is used to refer to amino acids in an amino acid sequence.

The term “variant” refers to a polypeptide that has an equivalent function to the amino acid sequences described herein, but which includes one or more amino acid substitutions, insertions or deletions.

The terms “selectively binds/selectively binding” and “specifically binds/specifically binding” may be used interchangeably herein.

“Heavy chain variable region” or “VH” refers to the fragment of the heavy chain of an antigen-binding domain or antibody that contains three CDRs interposed between flanking stretches known as framework regions, which are more highly conserved than the CDRs and form a scaffold to support the CDRs. “Light chain variable region” or “VL” refers to the fragment of the light chain of an antigen-binding domain or antibody that contains three CDRs interposed between framework regions.

“Complementarity determining region” or “CDR” with regard to antigen-binding domain or antibody or antigen-binding fragment thereof refers to a highly variable loop in the variable region of the heavy chain of the light chain of an antibody. CDRs can interact with the antigen conformation and largely determine binding to the antigen (although some framework regions are known to be involved in binding). The heavy chain variable region and the light chain variable region each contain 3 CDRs (heavy chain CDRs 1, 2 and 3 and light chain CDRs 1, 2 and 3, numbered from the amino to the carboxy terminus).

It may be possible to introduce one or more mutations (substitutions, additions or deletions) into each CDR without negatively affecting CD160-binding activity. Each CDR may, for example, have one, two or three amino acid mutations.

The CDRs of the variable regions of a heavy and light chain of an antigen-binding domain or antibody can be predicted from the heavy and light chain variable region sequences of the antibody, using prediction software available in the art, e.g. using the Abysis algorithm, or using the IMGT/V-QUEST software, e.g. the IMGT algorithm (ImMunoGeneTics) which can be found at www.IMGT.org, (see for example Lefranc et al, 2009 NAR 37:D1006-D1012 and Lefranc 2003, Leukemia 17: 260-266). CDR regions identified by either algorithm are considered to be equally suitable for use in the invention. CDRs may vary in length, depending on the antigen-binding domain or antibody from which they are predicted and between the heavy and light chains. Thus, the three heavy chain CDRs of an intact antigen-binding domain or antibody may be of different lengths (or may be of the same length) and the three light chain CDRs of an intact antigen-binding domain or antibody may be of different lengths (or may be of the same length). A CDR for example, may range from 2 or 3 amino acids in length to 5, 6, 7, 8, 9, 10, 11, 12, 13, 14, 15, 16, 17, 18, 19 or 20 amino acids in length. Particularly, a CDR may be from 3-14 amino acids in length, e.g. at least 3 amino acids and less than 15 amino acids.

As used herein, “variant” is synonymous with “mutant” and refers to a polynucleotide or amino acid sequence which differs in comparison to the corresponding wild-type sequence. The term “wild-type” is used to mean a gene or protein having a polynucleotide or amino acid sequence respectively, which is identical with the native gene or protein respectively.

Identity comparisons can be conducted by eye, or more usually, with the aid of readily available sequence comparison programs. These commercially available computer programs can calculate % identity between two or more sequences. A suitable computer program for carrying out such an alignment is the GCG Wisconsin Bestfit package (University of Wisconsin, U.S.A.; Devereux et al., 1984, Nucleotide sequences Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching. For example, the percentage identity between two polypeptide sequences may be readily determined by BLAST which is freely available at http://blast.ncbi.nlm.nih.gov

Once the software has produced an optimal alignment, it is possible to calculate % identity. The software typically does this as part of the sequence comparison and generates a numerical result.

The sequence may have one or more deletions, insertions or substitutions of amino acid residues which produce a silent change and result in a functionally equivalent molecule. These sequences are encompassed by the present invention. Deliberate amino acid substitutions may be made on the basis of similarity in polarity, charge, solubility, hydrophobicity, hydrophilicity, and/or the amphipathic nature of the residues as long as the activity is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Suitably, the polypeptide comprising the CD160 binding domain may be an antibody or fragment thereof, a chimeric antigen receptor (CAR) or a bispecific T cell engager (BiTE).

In the context of a CAR or a BiTE, in order to stimulate T cell activation the CD160 binding domain may bind to its cognate antigen (CD160) with a certain binding profile (for example, with a required binding affinity).

### Antibody

In one aspect, the present invention provides an antibody or fragment thereof comprising a CD160 binding domain according to the present invention. Thus, the antibody or fragment thereof may be capable of selectively binding to CD160.

As used herein, “antibody” means a protein or polypeptide having an antigen binding site or antigen-binding domain which comprises at least one complementarity determining region CDR. The antibody may comprise 3 CDRs and have an antigen binding site which is equivalent to that of a domain antibody (dAb). The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen. The antibody may be a whole immunoglobulin molecule or a part thereof such as a Fab, F(ab)′₂, Fv, single chain Fv (ScFv) fragment, Nanobody or single chain variable domain (which may be a VH or VL chain, having 3 CDRs). The antibody may be a bifunctional antibody. The antibody may be non-human, chimeric, humanised or fully human.

Descriptions of an antibody of the present invention provided herein are generally applicable to an antigen binding fragment thereof.

The antibody may be a monoclonal antibody or a polyclonal antibody. Preferably, the antibody is a monoclonal antibody.

Examples of an antigen-binding fragment include, but are not limited to, a single chain antibody (scFv), a single-domain antibody (sdAb), an antigen-binding fragment (Fab), a camelid antibody (VHH), a variable region (Fv), a heavy chain variable region (VH), a light chain variable region (VL), and a complementarity determining region (CDR).

The antibody may be a full-length, classical antibody. For example the antibody may be an IgG, IgM or IgA molecule.

Suitably, the antibody is a full monoclonal antibody.

Antibodies may be obtained by techniques comprising immunizing an animal with a target antigen and isolating the antibody from serum. Monoclonal antibodies may be made by the hybridoma method first described by Kohler et al., Nature 256:495 (1975), or may be made by recombinant DNA methods (see, e.g., U.S. Pat. No. 4,816,567). The monoclonal antibodies may also be isolated from phage antibody libraries using the techniques described in Clackson et al., Nature 352:624-628 (1991) and Marks et al., J. Mol. Biol. 222:581-597 (1991), for example. The antibody may also be a chimeric or humanized antibody or fragment thereof.

The antibody of fragment according to the invention may prove useful in any method which relies on a high affinity binding interaction between an antigen-binding domain and a cognate target. Thus, the antibody of fragment according to the invention may be used as a detection antibody and/or a capture antibody. The antibody of fragment according to the invention may be used a therapeutic antibody, for example, as a therapeutic antibody that targets CD160 protein or a cell expressing CD160. A non-limiting example therefore for the application of the antibody of fragment according to the invention is the use in the treatment of cancers characterized by expression and/or overexpression of CD160.

The present invention also encompasses fragments of any antibody or protein or polypeptide as defined herein. It will be appreciated that a fragment comprises an amino acid sequence that is shorter than the full-length sequence of an antibody or protein or polypeptide, but retains full biological activity and/or antigenic nature of the full-length sequence of the antibody or protein or polypeptide. It will also be appreciated that said fragment retains the same binding affinity of the full-length sequence of the antibody or protein or polypeptide.

### Antibody Conjugate

Suitably, an antibody conjugate is provided, which comprises the antibody or fragment thereof of the invention and a cargo or payload component. The antibody conjugate may be an antibody-drug conjugate (ADC), which is a class of targeted therapeutics that improves both the selectivity and the cytotoxic activity of cancer drugs. Typically, ADCs have three components: (i) a monoclonal antibody conjugated to (ii) a linker, which is also conjugated to (iii) a drug or payload, such as a cytotoxic or chemotherapeutic drug. The cytotoxic or chemotherapeutic drug refers to a drug that is destructive to a cell and reduces the viability of the cell. Suitable cytotoxic or chemotherapeutic drugs will be known in the art.

Suitability, the antibody conjugate of the invention is a molecule composed of an antibody or fragment thereof described herein, linked (i.e. conjugated) to a biologically active cytotoxic payload or drug, such as an anticancer drug. The linker may be any appropriate linker known in the art. The person skilled in the art will known that such linkers are routinely used in the production of conjugate molecules and would be able to select an appropriate linker.

Such linkers typically have chemically reactive groups at each end. These linkers can form a covalent attachment between two molecules, e.g. the antibody or fragment thereof and the drug or payload. Thus, the antibody or fragment thereof and the drug or payload may both be covalently linked to a linker. Suitably, one region of the linker may bind to the antibody or fragment thereof and another region of the linker may bind to the drug or payload. The linker may form, for example, hydrazone, disulfide or amide bonds between the antibody or fragment thereof and/or the drug or payload.

### Chimeric Antigen Receptor

The present invention provides a chimeric antigen receptor (CAR) comprising a CD160 binding domain as defined herein.

Chimeric antigen receptors (CARs), also known as chimeric T cell receptors, artificial T cell receptors and chimeric immunoreceptors, are engineered receptors, which graft an arbitrary specificity onto an immune effector cell. In a classical CAR, the specificity of a monoclonal antibody is grafted on to a T cell. CAR-encoding nucleic acids may be transferred to T cells using, for example, retroviral vectors. In this way, a large number of cancer-specific T cells can be generated for adoptive cell transfer. Phase I clinical studies of this approach show efficacy.

The target-antigen binding domain of a CAR is commonly fused via a spacer and transmembrane domain to a signaling endodomain, wherein said signaling endodomain is capable of directly transducing an activation signal into the T cell activation signalling cascade. When the CAR binds the target-antigen, this results in the transmission of an activating signal to the T cell it is expressed on. Thus, the CAR of the present invention is able to activate the T cell it is expressed on following binding of the CD160 binding domain to CD160 expressed on the surface of target cells.

Suitably, the CD160 binding domain as defined herein may be fused via a spacer and transmembrane domain to a signaling endodomain.

### Transmembrane Domain

The CAR of the invention may also comprise a transmembrane domain which spans the membrane. The transmembrane domain may be any protein structure which is thermodynamically stable in a membrane It may comprise a hydrophobic alpha helix. The transmembrane domain may be derived from CD8, CD28 or human IgG.

The transmembrane domain may be derived from any type I transmembrane protein. The transmembrane domain may be a synthetic sequence predicted to form a hydrophobic helix.

As used herein, the term “derived from” refers to the origin or source, and may include naturally occurring, recombinant, unpurified, or purified molecules. The term “derived from” encompasses the terms “originated from,” “obtained from,” “obtainable from,” “isolated from,” and “created from.”

The transmembrane domain may comprise the sequence shown as SEQ ID NO: 65.

The transmembrane domain may comprise the sequence shown as SEQ ID NO: 66.

(CD8a transmembrane domain)
SEQ ID NO: 65
IYIWAPLAGTCGVLLLSLVIT
(CD28 transmembrane domain)
SEQ ID NO: 66
FWVLVVVGGVLACYSLLVTVAFIIFWV

The CAR of the invention may comprise a variant of the sequence shown as SEQ ID NO: 65 or 66 having at least 80% sequence identity, provided that the variant sequence retains the capacity to insert into and span the membrane.

The variant sequence may have at least 80%, 85%, 90%, 95%, 98% or 99% sequence identity to SEQ ID NO: 65, provided that the variant sequence retains the capacity to insert into and span the membrane.

The variant sequence may have at least 80%, 85%, 90%, 95%, 98% or 99% sequence identity to SEQ ID NO: 66, provided that the variant sequence retains the capacity to insert into and span the membrane.

### Intracellular Signalling Domain (Endodomain)

The endodomain is the signal-transmission portion of the CAR. After antigen recognition, receptors cluster and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28, 41-BB and OX40 can be used with CD3-Zeta to transmit a proliferative/survival signal, or all three can be used together.

The endodomain of the CAR of the present invention may comprise the CD28 endodomain and/or OX40 endodomain and/or 41-BB endodomain and/or CD3-Zeta endodomain.

The intracellular T cell signalling domain (endodomain) of the CAR of the present invention may comprise the sequence shown as SEQ ID NO: 67, 68, 69, 70, 71 or 72 or a variant thereof having at least 80% sequence identity.

(CD28 endodomain)
SEQ ID NO: 67
RSKRSRLLHSDYMNMTPRRPGPTRKHYQPYAPPRDFAAY
(OX40 endodomain)
SEQ ID NO: 68
RRDQRLPPDAHKPPGGGSFRTPIQEEQADAHSTLAKI
(41-BB endodomain)
SEQ ID NO: 69
KRGRKKLLYIFKQPFMRPVQTTQEEDGCSCRFPEEEEGGCEL
(CD3 zeta endodomain)
SEQ ID NO: 70
RSRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTA
TKDTYDALHMQALPPR
(CD28Z)
SEQ ID NO: 71
RSKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHM
QALPPR
(CD28OXZ)
SEQ ID NO: 72
RSKRSRLLHSDYMNMTPRRPGPTRKHYQPYAPPRDFAAYRSRDQRLPPD
AHKPPGGGSFRTPIQEEQADAHSTLAKIRVKFSRSADAPAYQQGQNQLY
NELNLGRREEYDVLDKRRGRDPEMGGKPRRKNPQEGLYNELQKDKMAEA
YSEIGMKGERRRGKGHDGLYQGLSTATKDTYDALHMQALPPR

A variant sequence may have at least 80%, 85%, 90%, 95%, 98% or 99% sequence identity to SEQ ID NO: 67, provided that the sequence provides an effective intracellular T cell signaling domain.

A variant sequence may have at least 80%, 85%, 90%, 95%, 98% or 99% sequence identity to SEQ ID NO: 68, provided that the sequence provides an effective intracellular T cell signaling domain.

A variant sequence may have at least 80%, 85%, 90%, 95%, 98% or 99% sequence identity to SEQ ID NO: 69, provided that the sequence provides an effective intracellular T cell signaling domain.

A variant sequence may have at least 80%, 85%, 90%, 95%, 98% or 99% sequence identity to SEQ ID NO: 70, provided that the sequence provides an effective intracellular T cell signaling domain.

A variant sequence may have at least 80%, 85%, 90%, 95%, 98% or 99% sequence identity to SEQ ID NO: 71, provided that the sequence provides an effective intracellular T cell signaling domain.

A variant sequence may have at least 80%, 85%, 90%, 95%, 98% or 99% sequence identity to SEQ ID NO: 72, provided that the sequence provides an effective intracellular T cell signaling domain.

### Signal Peptide

The CAR of the present invention may comprise a signal peptide so that when the CAR is expressed inside a cell, such as a T cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The CAR of the invention may have the general formula:

Signal peptide-CD160 binding domain-spacer domain-transmembrane domain-intracellular T cell signaling domain.

The signal peptide may comprise the SEQ ID NO: 73 or a variant thereof having 5, 4, 3, 2 or 1 amino acid mutations (insertions, substitutions or additions) provided that the signal peptide still functions to cause cell surface expression of the CAR.

SEQ ID NO: 73:
METDTLLLWVLLLWVPGSTG

The signal peptide of SEQ ID NO: 73 is compact and highly efficient. It is predicted to give about 95% cleavage after the terminal glycine, giving efficient removal by signal peptidase.

### Spacer

The CAR of the present invention may comprise a spacer sequence to connect the CD160 binding domain with the transmembrane domain and spatially separate the CD160 binding domain from the endodomain. A flexible spacer allows the CD160 binding domain to orient in different directions to enable CD160 binding.

The spacer sequence may, for example, comprise an IgG1 Fc region, an IgG1 hinge or a CD8 stalk, or a combination thereof. The spacer may alternatively comprise an alternative sequence which has similar length and/or domain spacing properties as an IgG1 Fc region, an IgG1 hinge or a CD8 stalk.

A human IgG1 spacer may be altered to remove Fc binding motifs.

Examples of amino acid sequences for these spacers are given below:

(hinge-CH2CH3 of human IgG1)
SEQ ID NO: 74
AEPKSPDKTHTCPPCPAPPVAGPSVFLFPPKPKDTLMIARTPEVTCVVVDVSHEDPEVKFNWYVDGVE
VHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGKKD
(human CD8 stalk):
SEQ ID NO: 75
TTTPAPRPPTPAPTIASQPLSLRPEACRPAAGGAVHTRGLDFACDI
(human IgG1 hinge):
SEQ ID NO: 76
AEPKSPDKTHTCPPCPKDPK
(IgG1 Hinge-Fc)
SEQ ID NO: 77
AEPKSPDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGKKDPK
(IG1 Hinge-Fc modified to remove Fc receptor recognition motifs)
SEQ ID NO: 78
AEPKSPDKTHTCPPCPAPPVA*GPSVFLFPPKPKDTLMIARTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGKKDPK

Modified residues are underlined; * denotes a deletion.

A variant sequence may have at least 80%, 85%, 90%, 95%, 98% or 99% sequence identity to SEQ ID NO: 74.

A variant sequence may have at least 80%, 85%, 90%, 95%, 98% or 99% sequence identity to SEQ ID NO: 75.

A variant sequence may have at least 80%, 85%, 90%, 95%, 98% or 99% sequence identity to SEQ ID NO: 76.

A variant sequence may have at least 80%, 85%, 90%, 95%, 98% or 99% sequence identity to SEQ ID NO: 77.

A variant sequence may have at least 80%, 85%, 90%, 95%, 98% or 99% sequence identity to SEQ ID NO: 78.

### CD160 CARs

The CAR of the present invention may comprise the sequence shown as SEQ ID NO: 79 or a variant thereof which has at least 80%, 85%, 90%, 95%, 98% or 99% sequence identity thereto but retains the capacity to i) bind CD160 and ii) induce T cell signalling.

The CAR of the present invention may comprise the sequence shown as SEQ ID NO: 80 or a variant thereof which has at least 80%, 85%, 90%, 95%, 98% or 99% sequence identity thereto but retains the capacity to i) bind CD160 and ii) induce T cell signalling.

The CAR of the present invention may comprise the sequence shown as SEQ ID NO: 81 or a variant thereof which has at least 80%, 85%, 90%, 95%, 98% or 99% sequence identity thereto but retains the capacity to i) bind CD160 and ii) induce T cell signalling.

The CAR may be a CD8STK-CD28OX40Zeta CAR. The CAR may be a CD8STK-41BBZeta CAR.

SEQ ID NO: 79
METDTLLLWVLLVWIPGSTGQVNLQESGPGLVQPSETLSLTCTVSGFSLTSYGVYWVRQPPGKGLEWM
GRIWNDGGTDYNPALKSRLSISRDTSKSQVFLKLNSLQTEDTAIYFCARGWTHWGQGTLVTVSSGGGG
SGGGGSGGGGSDIQMTQSPASLSASLGETVSIECLASEDIYNNLAWYQQKPGKSPQLLIYYASSLQDG
VPSRFSGSGSGTQYSLKINSLESEDVATYFCLQDSEYPPTFGGGTKLELKRSDPTTTPAPRPPTPAPT
IASQPLSLRPEACRPAAGGAVHTRGLDFACDIFWVLVVVGGVLACYSLLVTVAFIIFWVRSKRSRLLH
SDYMNMTPRRPGPTRKHYQPYAPPRDFAAYRSRDQRLPPDAHKPPGGGSFRTPIQEEQADAHSTLAKI
RVKFSRSADAPAYQQGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR
SEQ ID NO: 80
METDTLLLWVLLLWVPGSTGEVKLVESGGGLVQPGRPLKLSCAASGFTESNYGMAWVRQAPKKGLEWV
ATISTGDDNTYYRDSVKGRFTISRDNAKNTQYLQMDSLRSEDTATYYCATHNYESTIFDYWGQGVMVT
VSSGGGGSGGGGSGGGGSDIAITQSPASMTASPGERVTITCRASSSVSYMHWYQQKPGASPKPWIYET
SKLASGVPDRESGSGSGTSYSLTINNMEAEDAATYYCQQWNYPSMYTFGAGTKLELKRSDPTTTPAPR
PPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKESRSADAPAYQQGQNQLYNELNLGRREEY
DVLDKRRGRDPEMGGKPRRKNPQEGLYNELQKDKMAEAYSEIGMKGERRRGKGHDGLYQGLSTATKDT
YDALHMQALPPR
SEQ ID NO: 81
METDTLLLWVLLLWVPGSTGEVQLVESGGGFVQPGGSLKLSCAASGFTFSNYGMAWVRQAPKKGLEWV
ASISSGRGNTYYRDSVKGRFTISRDNAKNTQYLQMDSLRSEDTATYYCVTHNYESTIFDYWGQGVMVT
VSSGGGGSGGGGSGGGGSEIVLTQSPTTMTASPGEKVTIICRASSSVSYMHWYQQKPGASPKPWIYET
SKLASGVPDRESGSGSGTSYSLTENNMEAEDAATYYCQQWNYPSMYTFGAGTKLELKRSDPTTTPAPR
PPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKESRSADAPAYQQGQNQLYNELNLGRREEY
DVLDKRRGRDPEMGGKPRRKNPQEGLYNELQKDKMAEAYSEIGMKGERRRGKGHDGLYQGLSTATKDT
YDALHMQALPPR

### Suicide Genes

Since T cells engraft and are autonomous, a means of selectively deleting CAR T cells in recipients of anti-CD160 CAR T cells is desirable. Suicide genes are genetically encodable mechanisms which result in selective destruction of infused T cells in the face of unacceptable toxicity. The earliest clinical experience with suicide genes is with the Herpes Virus Thymidine Kinase (HSV-TK) which renders T cells susceptible to Ganciclovir. HSV-TK is a highly effective suicide gene. However, pre-formed immune responses may restrict its use to clinical settings of considerable immunosuppression such as haploidentical stem cell transplantation. Inducible Caspase 9 (iCasp9) is a suicide gene constructed by replacing the activating domain of Caspase 9 with a modified FKBP12. iCasp9 is activated by an otherwise inert small molecular chemical inducer of dimerization (CID). iCasp9 has been recently tested in the setting of haploidentical HSCT and can abort GvHD. The biggest limitation of iCasp9 is dependence on availability of clinical grade proprietary CID. Both iCasp9 and HSV-TK are intracellular proteins, so when used as the sole transgene, they have been co-expressed with a marker gene to allow selection of transduced cells.

An iCasp9 may comprise the sequence shown as SEQ ID NO: 82 or a variant thereof having at least 80%, 85%, 90%, 95%, 98% or 99% sequence identity.

SEQ ID NO: 82
MLEGVQVETISPGDGRTFPKRGQTCVVHYTGMLEDGKKVDSSRDRNKPFKEMLGKQEVIRGWEEGVAQ
MSVGQRAKLTISPDYAYGATGHPGIIPPHATLVEDVELLKLESGGGSGVDGEGDVGALESLRGNADLA
YILSMEPCGHCLIINNVNFCRESGLRTRTGSNIDCEKLRRRFSSLHEMVEVKGDLTAKKMVLALLELA
QQDHGALDCCVVVILSHGCQASHLQFPGAVYGTDGCPVSVEKIVNIENGTSCPSLGGKPKLFFIQACG
GEQKDHGFEVASTSPEDESPGSNPEPDATPFQEGLRTFDQLDAISSLPTPSDIFVSYSTFPGFVSWRD
PKSGSWYVETLDDIFEQWAHSEDLQSLLLRVANAVSVKGIYKQMPGCENELRKKLFFKTSAS

The present inventors have recently described a novel marker/suicide gene known as RQR8 which can be detected with the antibody QBEnd10 and expressing cells lysed with the therapeutic antibody Rituximab.

An RQR8 may comprise the sequence shown as SEQ ID NO: 83 or a variant thereof having at least 80%, 85%, 90%, 95%, 98% or 99% sequence identity.

SEQ ID NO: 83
MGTSLLCWMALCLLGADHADACPYSNPSLCSGGGGSELPTQGTFSNVST
NVSPAKPTTTACPYSNPSLCSGGGGSPAPRPPTPAPTIASQPLSLRPEA
CRPAAGGAVHTRGLDFACDIYIWAPLAGTCGVLLLSLVITLYCNHRNRR
RVCKCPRPVV

The suicide gene may be expressed as a single polypeptide with the CAR, for example by using a self-cleaving peptide between the two sequences.

### Bi-Specific T Cell Engagers (Bites)

In one embodiment, the present invention provides a T cell activator molecule which is a bi-specific molecule (i.e. a bi-specific T cell engager (BiTE)) which comprises a CD160 binding domain as described herein as a first domain, and a T cell activating domain as a second domain.

Bi-specific T cell engaging molecules are a class of bi-specific antibody-type molecules that have been developed, primarily for the use as anti-cancer drugs. They direct a host's immune system, more specifically the T cells' cytotoxic activity, against a target cell, such as a cancer cell. In these molecules, one binding domain binds to binds to a T cell via, for example, the CD3 receptor, and the other to a target cells such as a tumor cell (via a tumor specific molecule). Since the bispecific molecule binds both the target cell and the T cell, it brings the target cell into proximity with the T cell, so that the T cell can exert its effect, for example, a cytotoxic effect on a cancer cell. The formation of the T cell:bispecific Ab:cancer cell complex induces signaling in the T cell leading to, for example, the release of cytotoxic mediators. Ideally, the agent only induces the desired signaling in the presence of the target cell, leading to selective killing.

Thus, a bi-specific molecule of the present invention brings a CD160-expressing cell (for example, a CD160+CLL cell) into proximity with a T cell, so that the T cell can exert its effect on the CLL cell. The requirement of co-localisation via binding of the CD160 bi-specific molecule leads to selective killing of CD160-positive cells. In other words, a bi-specific molecule of the present invention is able to activate T cells following binding of the CD160 binding domain to CD160 expressed on the surface of target cells.

BiTEs are commonly made by fusing an anti-CD3 scFv to an anti-target antigen scFv via a short five residue peptide linker (GGGGS).

### T Cell Activing Domain

The second domain of the bi-specific molecule of the present invention is capable of activating T cells. T cells have a T cell-receptor (TCR) at the cell surface which recognises antigenic peptides when presented by an MHC molecule on the surface of an antigen presenting cell.

Such antigen recognition results in the phosphorylation of immunoreceptor tyrosine-based activation motifs (ITAMs) by Src family kinases, triggering recruitment of further kinases which results in T cell activation including Ca²⁺ release.

The second domain may cause T cell activation by triggering the same pathway triggered by antigen-specific recognition by the TCR. Thus, the second domain may induce T cell signalling

### Cluster of Differentiation 3 (CD3)

The second domain of the bi-specific molecule of the invention may bind CD3.

CD3 is a protein complex composed of four distinct chains: a CD3γ chain, a CD3δ chain, and two CD3ε chains. CD3 associates with the T cell receptor (TCR) and the ζ-chain on the surface of a T cell to generate an activation signal. The TCR, ζ-chain, and CD3 molecule together comprise the TCR complex.

Clustering of CD3 on T cells, e.g. by immobilized anti-CD3-antibodies, leads to T cell activation similar to the engagement of the T cell receptor, but independent from its clone typical specificity.

Due to its central role in modulating T cell activity, there have been attempts to develop molecules that are capable of binding TCR/CD3. Much of this work has focused on the generation of antibodies that are specific for the human CD3 antigen.

The second domain may comprise an antibody or part thereof which specifically binds CD3, such as OKT3, WT32, anti-leu-4, UCHT-1, SPV-3TA, TR66, SPV-T3B or affinity tuned variants thereof.

As used herein, “antibody” means a polypeptide having an antigen binding site which comprises at least one complementarity determining region CDR. The antibody may comprise 3 CDRs and have an antigen binding site which is equivalent to that of a domain antibody (dAb). The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen. The antibody may be a whole immunoglobulin molecule or a part thereof such as a Fab, F(ab)′₂, Fv, single chain Fv (ScFv) fragment, Nanobody or single chain variable domain (which may be a VH or VL chain, having 3 CDRs). The antibody may be a bifunctional antibody. The antibody may be non-human, chimeric, humanised or fully human.

Alternatively the second domain may comprise a CD3-binding molecule which is not derived from or based on an immunoglobulin. A number of “antibody mimetic” designed repeat proteins (DRPs) have been developed to exploit the binding abilities of non-antibody polypeptides. Such molecules include ankyrin or leucine-rich repeat proteins e.g. DARPins (Designed Ankyrin Repeat Proteins), Anticalins, Avimers and Versabodies.

The second domain of the bi-specific molecule of the invention may comprise all or part of the monoclonal antibody OKT3, which was the first monoclonal antibody approved by the FDA. OKT3 is available from ATCC CRL 8001. The antibody sequences are published in U.S. Pat. No. 7,381,803.

The second domain may comprise one or more CDRs from OKT3. The second binding domain may comprise CDR3 from the heavy-chain of OKT3 and/or CDR3 from the light chain of OKT3. The second binding domain may comprise all 6 CDRs from OKT3, as shown below.

Heavy Chain
CDR1:
(SEQ ID NO: 84)
KASGYTFTRYTMH
CDR2:
(SEQ ID NO: 85)
INPSRGYTNYNQKFKD
CDR3:
(SEQ ID NO: 86)
YYDDHYCLDY
Light Chain
CDR1:
(SEQ ID NO: 87)
SASSSVSYMN
CDR2:
(SEQ ID NO: 88)
RWIYDTSKLAS
CDR3:
(SEQ ID NO: 89)
QQWSSNPFT

Suitably, one or more of the CDRs may comprise comprises one, two or three amino acid mutations.

The second binding domain may comprise a scFv which comprises the CDR sequences from OKT3. The second binding domain may comprise the scFv sequence shown below as SEQ ID NO: 90 or a variant thereof having at least 80%, 85%, 90%, 95%, 98% or 99% sequence identity thereto, which retains the capacity to bind CD3.

SEQ ID NO: 90
QVQLQQSGAELARPGASVKMSCKASGYTFTRYTMHWVKQRPGQGLEWIGYINPSRGYTNYNQKEKDKA
TLTTDKSSSTAYMQLSSLTSEDSAVYYCARYYDDHYCLDYWGQGTTLTVSSSGGGGSGGGGSGGGGSQ
IVLTQSPAIMSASPGEKVTMTCSASSSVSYMNWYQQKSGTSPKRWIYDTSKLASGVPAHERGSGSGTS
YSLTISGMEAEDAATYYCQQWSSNPFTFGSGTKLEINR

The second domain may comprise one or more CDRs from UCHT1. The second binding domain may comprise CDR3 from the heavy-chain of UCHT1 and/or CDR3 from the light chain of UCHT1. The second binding domain may comprise all 6 CDRs from UCHT1, as shown below.

Heavy Chain
CDR1:
(SEQ ID NO: 91)
GYSFTGYT
CDR2:
(SEQ ID NO: 92)
INPYKGVS
CDR3:
(SEQ ID NO: 93)
ARSGYYGDSDWYFDV
Light Chain
CDR1:
(SEQ ID NO: 94)
QDIRNY
CDR2:
(SEQ ID NO: 95)
YTS
CDR3:
(SEQ ID NO: 96)
QQGNTLPWT

Suitably, one or more of the CDRs may comprise comprises one, two or three amino acid mutations.

The second binding domain may comprise a scFv which comprises the CDR sequences from UCHT1. The second binding domain may comprise the scFv sequence shown below as SEQ ID NO: 97 or a variant thereof having at least 80%, 85%, 90%, 95%, 98% or 99% sequence identity thereto, which retains the capacity to bind CD3.

SEQ ID NO: 97
DIQMTQSPSSLSASVGNRVTITCRASQDIRNYLNWYQQKPGKAPKLLIYYTSRLESGVPSRFSGSGSG
TDYTLTISSLQPEDFATYYCQQGNTLPWTFGQGTKVEIKSGGGGSGGGGSGGGGSEVQLVESGGGLVQ
PGGSLRLSCAASGYSFTGYTMNWVRQAPGKGLEWVALINPYKGVSTYNQKFKDRFTISVDKSKNTAYL
QMNSLRAEDTAVYYCARSGYYGDSDWYFDVWGQGTLVTVSS

The second domain may comprise one or more CDRs from YTH. The second binding domain may comprise CDR3 from the heavy-chain of YTH and/or CDR3 from the light chain of YTH. The second binding domain may comprise all 6 CDRs from YTH, as shown below.

Heavy Chain
CDR1:
(SEQ ID NO: 98)
GFTFSSFP
CDR2:
(SEQ ID NO: 99)
ISTSGGRT
CDR3:
(SEQ ID NO: 100)
AKFRQYSGGFDY
Light Chain
CDR1:
(SEQ ID NO: 101)
SGNIENNY
CDR2:
(SEQ ID NO: 102)
DDD
CDR3:
(SEQ ID NO: 103)
HSYVSSFNV

Suitably, one or more of the CDRs may comprise comprises one, two or three amino acid mutations.

The second binding domain may comprise a scFv which comprises the CDR sequences from YTH. The second binding domain may comprise the scFv sequence shown below as SEQ ID NO: 104 or a variant thereof having at least 80%, 85%, 90%, 95%, 98% or 99% sequence identity thereto, which retains the capacity to bind CD3.

SEQ ID NO: 104
DEMLTQPHSVSESPGKTVIISCTLSSGNIENNYVHWYQQRPGRAPTTVIFDDDKRPDGVPDRFSGSID
RSSNSASLTISGLQTEDEADYYCHSYVSSENVEGGGTKLTVLSGGGGSGGGGSGGGGSEVQLLESGGG
LVQPGGSLRLSCAASGFTFSSFPMAWVRQAPGKGLEWVSTISTSGGRTYYRDSVKGRFTISRDNSKNT
LYLQMNSLRAEDTAVYYCAKFRQYSGGFDYWGQGTLVTVSS

A variant sequence of SEQ ID NOs: 90, 97 or 104 may have at least 80%, 85%, 90%, 95%, 98% or 99% sequence identity and have equivalent or improved CD3 binding and/or TCR activation capabilities compared to the sequence shown as SEQ ID NO: 90, 97 or 104.

### Secretion Signal Peptide

The bi-specific molecule of the invention may comprise a signal peptide to aid in its production. The signal peptide may cause the bi-specific molecule to be secreted by a host cell, such that the bi-specific molecule can be harvested from the host cell supernatant.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The bi-specific molecule may have the general formula:

Signal peptide-first domain-second domain.

The signal peptide may comprise the SEQ ID NO: 105 or 106 or a variant thereof having 5, 4, 3, 2 or 1 amino acid mutations (insertions, substitutions or additions) provided that the signal peptide still functions to cause secretion of the bi-specific molecule.

SEQ ID NO: 105:
METDTLLLWVLLLWVPGSTG
SEQ ID NO: 106:
MGTSLLCWMALCLLGADHADG

The signal peptides of SEQ ID NO: 105 and 106 are compact and highly efficient. They are predicted to give about 95% cleavage after the terminal glycine, giving efficient removal by signal peptidase.

### Spacer

The bi-specific molecule of the present invention may comprise a spacer or linker sequence to connect the first domain with the second domain and spatially separate the two domains.

The spacer sequence may, for example, comprise an IgG1 hinge or a CD8 stalk. The spacer or linker may alternatively comprise an alternative linker sequence which has similar length and/or domain spacing properties as an IgG1 hinge or a CD8 stalk.

The spacer may be a short spacer, for example a spacer which comprises less than 100, less than 80, less than 60 or less than 45 amino acids. The spacer may be or comprise an IgG1 hinge or a CD8 stalk or a modified version thereof.

Examples of amino acid sequences for these linkers are given below:

SEQ ID NO: 107 (IgG1 hinge):
AEPKSPDKTHTCPPCPKDPKSGGGGS
SEQ ID NO: 108 (CD8 stalk):
TTTPAPRPPTPAPTIASQPLSLRPEACRPAAGGAVHTRGLDFACD

The CD8 stalk has a sequence such that it may induce the formation of homodimers. If this is not desired, one or more cysteine residues may be substituted or removed from the CD8 stalk sequence. The bispecific molecule of the invention may include a spacer which comprises or consists of the sequence shown as SEQ ID NO: 107 or 108 or a variant thereof having at least 80%, 85%, 90%, 95%, 98% or 99% sequence identity, provided that the variant sequence is a molecule which causes approximately equivalent spacing of the first and second domains and/or that the variant sequence causes homodimerisation of the bi-specific molecule.

The bi-specific molecule of the invention may have the general formula:

Signal peptide-first domain-spacer-second domain.

The spacer may also comprise one or more linker motifs to introduce a chain-break. A chain break separate two distinct domains but allows orientation in different angles. Such sequences include the sequence SDP, and the sequence SGGGSDP (SEQ ID NO: 109).

The linker may comprise a serine-glycine linker, such as SGGGGS (SEQ ID NO: 113).

### CD160 BiTEs

The bi-specific molecule of the present invention may comprise the sequence shown as SEQ ID NO: 110 or a variant thereof which has at least 80%, 85%, 90%, 95%, 98% or 99% sequence identity thereto but retains the capacity to i) bind CD160 and ii) induce T cell signalling.

The bi-specific molecule of the present invention may comprise the sequence shown as SEQ ID NO: 111 or a variant thereof which has at least 80%, 85%, 90%, 95%, 98% or 99% sequence identity thereto but retains the capacity to i) bind CD160 and ii) induce T cell signalling.

The bi-specific molecule of the present invention may comprise the sequence shown as SEQ ID NO: 112 or a variant thereof which has at least 80%, 85%, 90%, 95%, 98% or 99% sequence identity thereto but retains the capacity to i) bind CD160 and ii) induce T cell signalling.

SEQ ID NO: 110
METDTLLLWVLLVWIPGSTGQVNLQESGPGLVQPSETLSLTCTVSGFSLTSYGVYWVRQPPGKGLEWM
GRIWNDGGTDYNPALKSRLSISRDTSKSQVELKLNSLQTEDTAIYFCARGWTHWGQGTLVTVSSGGGG
SGGGGSGGGGSDIQMTQSPASLSASLGETVSIECLASEDIYNNLAWYQQKPGKSPQLLIYYASSLQDG
VPSRFSGSGSGTQYSLKINSLESEDVATYFCLQDSEYPPTFGGGTKLELKRSDPTTTPAPRPPTPAPT
IASQPLSLRPEACRPAAGGAVHTRGLDFACDSGGGGSDEMLTQPHSVSESPGKTVIISCTLSSGNIEN
NYVHWYQQRPGRAPTTVIFDDDKRPDGVPDRFSGSIDRSSNSASLTISGLQTEDEADYYCHSYVSSEN
VFGGGTKLTVLSGGGGSGGGGSGGGGSEVQLLESGGGLVQPGGSLRLSCAASGFTFSSFPMAWVRQAP
GKGLEWVSTISTSGGRTYYRDSVKGRFTISRDNSKNTLYLQMNSLRAEDTAVYYCAKFRQYSGGFDYW
GQGTLVTVSS
SEQ ID NO: 111
METDTLLLWVLLVWIPGSTGEVKLVESGGGLVQPGRPLKLSCAASGFTESNYGMAWVRQAPKKGLEWV
ATISTGDDNTYYRDSVKGRFTISRDNAKNTQYLQMDSLRSEDTATYYCATHNYESTIFDYWGQGVMVT
VSSGGGGSGGGGSGGGGSDIAITQSPASMTASPGERVTITCRASSSVSYMHWYQQKPGASPKPWIYET
SKLASGVPDRESGSGSGTSYSLTINNMEAEDAATYYCQQWNYPSMYTFGAGTKLELKRSDPTTTPAPR
PPTPAPTIASQPLSLRPEACRPAAGGAVHTRGLDFACDSGGGGSDFMLTQPHSVSESPGKTVIISCTL
SSGNIENNYVHWYQQRPGRAPTTVIFDDDKRPDGVPDRFSGSIDRSSNSASLTISGLQTEDEADYYCH
SYVSSFNVFGGGTKLTVLSGGGGSGGGGSGGGGSEVQLLESGGGLVQPGGSLRLSCAASGFTFSSFPM
AWVRQAPGKGLEWVSTISTSGGRTYYRDSVKGRFTISRDNSKNTLYLQMNSLRAEDTAVYYCAKFRQY
SGGFDYWGQGTLVTVSS
SEQ ID NO: 112
METDTLLLWVLLVWIPGSTGEVQLVESGGGFVQPGGSLKLSCAASGFTFSNYGMAWVRQAPKKGLEWV
ASISSGRGNTYYRDSVKGRFTISRDNAKNTQYLQMDSLRSEDTATYYCVTHNYESTIFDYWGQGVMVT
VSSGGGGSGGGGSGGGGSEIVLTQSPTTMTASPGEKVTIICRASSSVSYMHWYQQKPGASPKPWIYET
SKLASGVPDRESGSGSGTSYSLTENNMEAEDAATYYCQQWNYPSMYTFGAGTKLELKRSDPTTTPAPR
PPTPAPTIASQPLSLRPEACRPAAGGAVHTRGLDFACDSGGGGSDEMLTQPHSVSESPGKTVIISCTL
SSGNIENNYVHWYQQRPGRAPTTVIFDDDKRPDGVPDRFSGSIDRSSNSASLTISGLQTEDEADYYCH
SYVSSFNVFGGGTKLTVLSGGGGSGGGGSGGGGSEVQLLESGGGLVQPGGSLRLSCAASGFTFSSFPM
AWVRQAPGKGLEWVSTISTSGGRTYYRDSVKGRFTISRDNSKNTLYLQMNSLRAEDTAVYYCAKFRQY
SGGFDYWGQGTLVTVSS

### Polynucleotide

In an aspect the present invention provides a nucleic acid sequence which encodes a CD160 binding domain of the present invention.

In one aspect the present invention provides a nucleic acid sequence which encodes an antibody or fragment thereof of the present invention.

In one aspect the present invention provides a nucleic acid sequence which encodes a CAR of the present invention.

In one aspect the present invention provides a nucleic acid sequence which encodes a bi-specific molecule of the present invention.

Due to the redundancy of the genetic code, variations in nucleic acid sequences are possible that encode for the same polypeptide. These sequences are encompassed by the present invention. Therefore multiple polynucleotides are envisaged, each with a different nucleic acid sequence but which encodes a polypeptide according to the invention or a further polypeptide as described herein. It is possible to design and produce such nucleic acid sequences without difficulty.

The nucleic acid sequence may be an RNA or DNA sequence or a variant thereof. The term “polynucleotide” includes an RNA or DNA sequence. It may be single or double stranded. It may, for example, be genomic, recombinant, mRNA or cDNA.

The nucleotide sequence may be codon optimised for production in the host cell of choice.

### Vector

The present invention also provides a vector which comprises a nucleic acid sequence according to the present invention. For example, the vector of the invention may comprise a polynucleotide comprising a nucleic acid sequence that encodes a molecule of the invention, such as an antibody or fragment thereof of the invention, or a CAR of the invention or a bi-specific molecule of the invention. Such a vector may be used to introduce the nucleic acid sequence into a host cell so that it expresses and produces a molecule of the invention.

The vector may be any agent capable of delivering or maintaining nucleic acid in a host cell, and includes viral vectors, plasmids, naked nucleic acids, nucleic acids complexed with polypeptide or other molecules and nucleic acids immobilised onto solid phase particles. The vector may, for example, be a plasmid or a viral vector, such as a retroviral vector or a lentiviral vector.

The vector may comprise the nucleic acid sequence encoding the CD160 binding domain according to the invention, operably linked to a heterologous sequence, such as a promoter or regulatory sequence. In general, it is advantageous to employ a strong promoter functional in eukaryotic cells. The strong promoter may be, but not limited to, the immediate early cytomegalovirus promoter (CMV-IE) of human or murine origin, or optionally having another origin such as the rat or guinea pig.

In more general terms, the promoter has either a viral, or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa et al., 2000), or the actin promoter (Miyazaki et al., 1989).

The promoter may be constitutive promoter. The promoter may be a tissue specific promoter.

The vector may be capable of transfecting or transducing a cell. In one aspect, the vector may be capable of transfecting or transducing a T cell.

The vector may also comprise a nucleic acid sequence encoding a suicide gene, such as iCasp9 or RQR8.

### Cell

The invention also provides a host cell which comprises a nucleic acid or a vector according to the invention.

The host cell may be capable of producing an antibody or fragment thereof of the invention. The host cell may be capable of producing a bi-specific molecule of the invention. The host cell may be capable of producing and/or expressing and/or may comprise a CAR of the invention.

The host cell may be a bacterial, fungal, yeast, plant or animal cell. Suitably the antibody or fragment thereof of the invention or a bi-specific molecule of the invention may be produced in a bacterial, fungal, yeast, plant or animal cell. Suitably, the host cell may be a mammalian cell, such as the human embryonic kidney cell line 293.

The cell may be a cytolytic immune cell such as a T cell or natural killer (NK) cell. Suitably the T cell or NK cell may produce and/or express and/or comprise the CAR of the invention. Suitably, the host cell may be a T cell. Suitably, the host cell may be a NK cell.

A T cell capable of expressing a CAR according to the invention may be made by transducing or transfecting a T cell with CAR-encoding nucleic acid. A NK cell capable of expressing a CAR according to the invention may be made by transducing or transfecting a NK cell with a CAR-encoding nucleic acid.

The CAR T cell or CAR NK cell may be generated ex vivo. The T cell or NK cell may be from a peripheral blood mononuclear cell (PBMC) sample from the patient or a donor. T cells may be activated and/or expanded prior to being transduced with CAR-encoding nucleic acid, for example by treatment with an anti-CD3 monoclonal antibody.

The present invention also provides a method for making a cell according to the invention, which comprises the step of introducing a polynucleotide according to the invention or a vector according to the invention into said cell. Said cell is then capable of expressing and/or producing an antibody or fragment thereof of the invention, or a CAR of the invention or a bi-specific molecule of the invention, when the host cell is cultured under conditions suitable for production of the molecule. The molecule can then be harvested from the host cell or supernatant.

Bi-specific molecules of the invention produced in a cell as set out above can be produced either intracellularly (e.g. in the cytosol, in the periplasma or in inclusion bodies) and then isolated from the host cells and optionally further purified; or they can be produced extracellularly (e.g. in the medium in which the host cells are cultured) and then isolated from the culture medium and optionally further purified.

### Pharmaceutical Composition

The present invention also relates to a pharmaceutical composition comprising a polynucleotide or a vector of the invention together with a pharmaceutically acceptable carrier, diluent or excipient, and optionally one or more further pharmaceutically active polypeptides and/or compounds.

The present invention also relates to a pharmaceutical composition comprising an antibody or fragment thereof of the invention together with a pharmaceutically acceptable carrier, diluent or excipient, and optionally one or more further pharmaceutically active polypeptides and/or compounds.

The present invention also relates to a pharmaceutical composition comprising a CAR-expressing cell of the invention together with a pharmaceutically acceptable carrier, diluent or excipient, and optionally one or more further pharmaceutically active polypeptides and/or compounds.

The present invention also relates to a pharmaceutical composition comprising a bi-specific molecule of the invention together with a pharmaceutically acceptable carrier, diluent or excipient, and optionally one or more further pharmaceutically active polypeptides and/or compounds.

Such formulations may, for example, be in a form suitable for intravenous infusion.

### Medical Use

The present invention provides an antibody or fragment thereof of the invention for use as a medicament in the treatment of a disease.

The present invention provides a cell of the invention expressing a CAR of the invention for use as a medicament in the treatment of a disease.

The present invention provides a bi-specific molecule of the invention for use as a medicament in the treatment of a disease.

The present invention provides a vector of the invention for use as a medicament in the treatment of a disease.

The present invention provides a pharmaceutical composition of the invention for use as a medicament in the treatment of a disease.

Suitably the disease may be a cancerous disease, in particular a cancerous disease associated with CD160 expression. The cancer may be selected from: NK lymphoma, TCRγδ lymphoma, leukaemia, chronic lymphocytic leukaemia (CLL), or hairy cell leukaemia.

The cancer may be a leukaemia.

The cancer may be CLL or hairy cell leukaemia.

In one aspect, the disease may be associated with neoangiogenesis.

Suitably, the disease associated with neoangiogenesis may be a cancer. The disease associated with neoangiogenesis may be a colon carcinoma or a melanoma. The disease associated with neoangiogenesis may be a neovascular disease, such as a neovascular eye disease.

Suitably, the antibody or fragment thereof of the invention, the cell expressing a CAR of the present invention, the bi-specific molecule of the present invention, the vector of the invention and/or the pharmaceutical composition of the invention may be used for the treatment of a cancerous disease associated with CD160 expression.

Suitably, the antibody or fragment thereof of the invention, the cell expressing a CAR of the present invention, the bi-specific molecule of the present invention, the vector of the invention and/or the pharmaceutical composition of the invention may be used for the treatment of CLL.

Cells expressing a CAR molecule of the present invention are capable of killing cancer cells, such as CLL cells. CAR-expressing cells may either be created ex vivo either from a patient's own peripheral blood (1^{st} party), or in the setting of a haematopoietic stem cell transplant from donor peripheral blood (2^{nd} party), or peripheral blood from an unconnected donor (3^{rd} party). Alternatively, CAR cells may be derived from ex-vivo differentiation of inducible progenitor cells or embryonic progenitor cells to T cells or NK cells. In these instances, CAR cells are generated by introducing DNA or RNA coding for the CAR by one of many means including transduction with a viral vector, transfection with DNA or RNA.

The present invention also relates to a method of treating a cancer or a disease associated with neoangiogenesis.

A method for the treatment of cancer may relate to the therapeutic use of an antibody or fragment thereof of the invention, a cell of the invention expressing a CAR of the invention, a bi-specific molecule of the invention, a vector of the invention and/or a pharmaceutical composition of the invention.

A method for the treatment of a disease associated with neoangiogenesis may relate to the therapeutic use of an antibody or fragment thereof of the invention, a cell of the invention expressing a CAR of the invention, a bi-specific molecule of the invention, a vector of the invention and/or a pharmaceutical composition of the invention.

In this respect, the antibody or fragment thereof, CAR-expressing cell, bi-specific molecule, vector and pharmaceutical composition may be administered to a subject having an existing disease or condition in order to lessen, reduce or improve at least one symptom associated with the disease and/or to slow down, reduce or block the progression of the disease. For example, the method of the invention may cause or promote T cell or NK cell mediated killing of CD160-expressing cells, such as cancer cells.

The present invention also relates to the use of an antibody or fragment thereof of the invention, a cell of the invention expressing a CAR of the invention, a bi-specific molecule of the invention, a vector of the invention and/or a pharmaceutical composition of the invention in the manufacture of a medicament for treating a cancer.

The present invention also relates to the use of an antibody or fragment thereof of the invention, a cell of the invention expressing a CAR of the invention, a bi-specific molecule of the invention, a vector of the invention and/or a pharmaceutical composition of the invention in the manufacture of a medicament for treating a disease associated with neoangiogenesis.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1

### Design of Degenerate Primer Library for Rat VH and VL Germline Genes

The IMGT database lists 232 Rattus Norvegicus germline heavy variable (VH) sequences with 13 families, and 164 kappa light variable (VLK) sequences in 21 families. 39 forward primers which anneal to the VH genes and 29 to the variable kappa light chains were designed. Predicted pseudogenes and not in-frame Open Reading Frames (ORFs) were excluded.

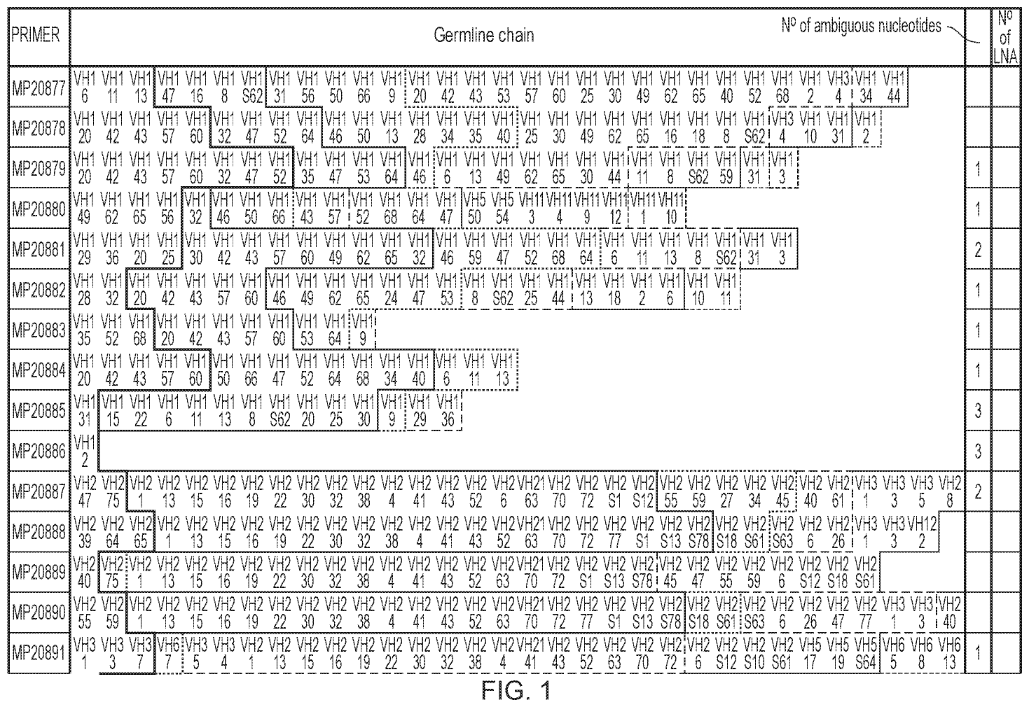

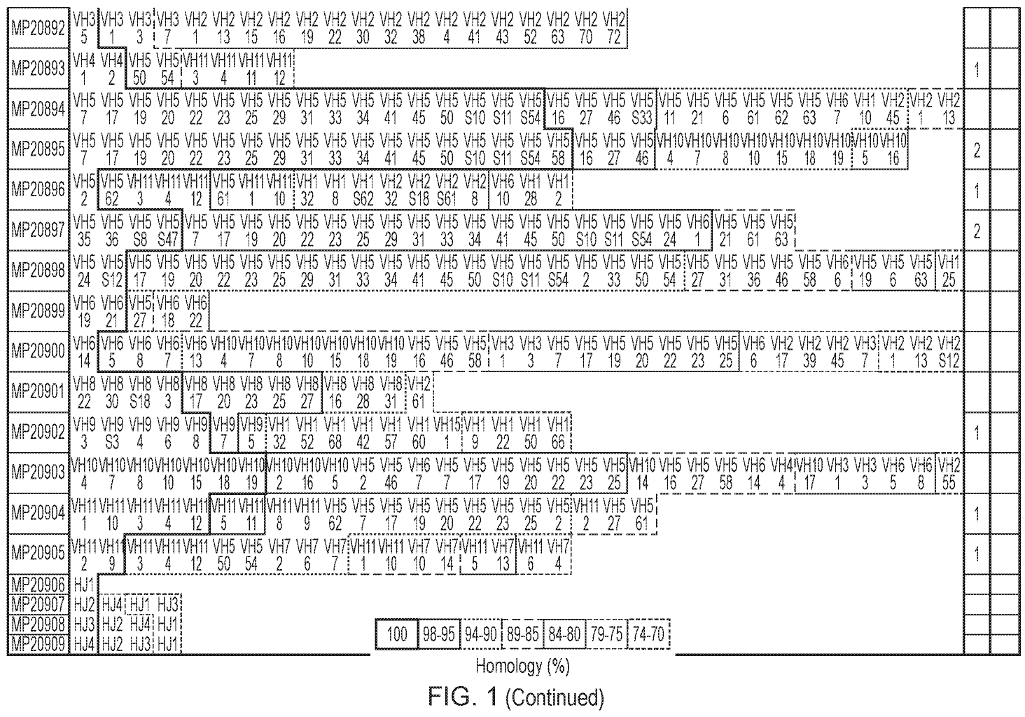

For VH regions, on average each primer amplified 23 variable genes, although smaller families required 1 or 2 primers (FIG. 1). An average of 19/subgroups were covered per primer. 4 reverse primers for VH [annealing to the heavy chain joining regions (HJ)] and 5 reverse primers for the VLK [annealing to the kappa chain joining regions (KJ)] were designed. Together these primers cover all the possible joining regions in rat VH and VLK.

The primer tail at the 5′ end of the VH gene and the 3′ end of the VL gene contained the annealing sites for the nested PCR outer primers and SfiI/NotI restriction sites for cloning into the phagemid vector (pHEN1). The tails at the 3′ end of the VH and the 5′ end of the VL included the serine-glycine linker sequence (3×GGGGS) as overlapping regions. The primary PCR amplification of VH and VL chains incorporated the outer tail regions. Overlap extension PCR created a single amplicon encoding an scFv in the VH-VL orientation separated by a serine-glycine linker sequence.

### Example 2

Primer-Set Effectively Amplifies VH and VL Genes without PCR-Biases

Deep-sequencing of VH/VLK genes from 5′RACE and primer-set amplified cDNA were studied and compared; cDNA was isolated from three wild type naïve, antigen unchallenged, Wistar rats. The material was used as template for both an Ig specific 5′RACE PCR and for primer-set amplification and sequenced using the Illumina MiSeq platform.

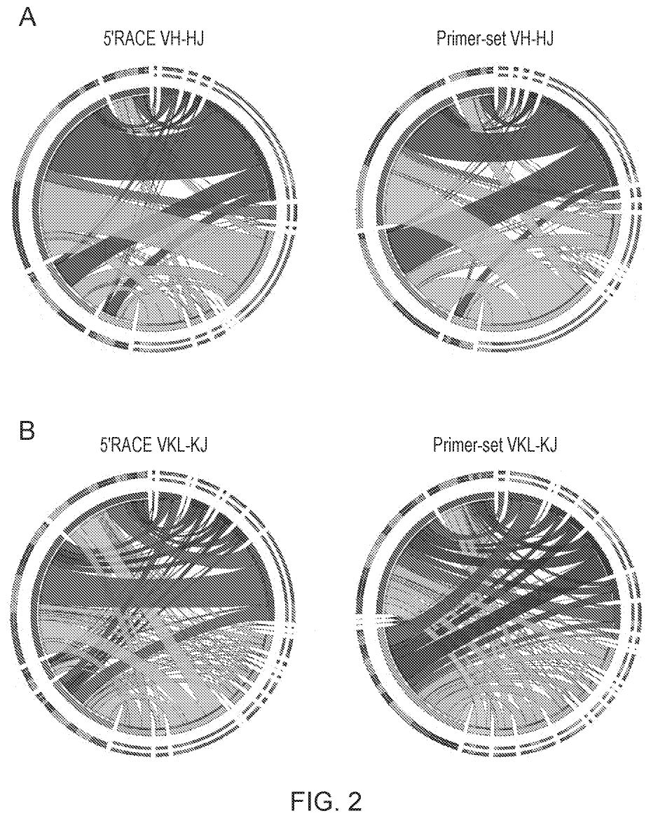

Analysis of the VH and VLK usage in naïve rat following 5′ RACE showed that VH family 5 was the most represented (39%) followed by family 2 and 1 with 28 and 16% respectively. The other families were all present at a lower frequency (1-4%) while family 12 and 15 were the least represented with a frequency of less than 0.1%. Analysis of VLK usage showed that family 22 and 12 and 1 were the most represented (28%, 20%, 12% of the total VLK usage respectively) while the majority of VKL families were present at similar frequencies between 3% to 5%. Families, 5, 9, 17, 19 and 21 were present at less than 1% and family 7, 13, 18 and were less than 0.1% (FIG. 2).

Next, the 5′RACE and primer-set amplified sequences were compared. The relative frequency of variable heavy (VH) and joining (JH) genes was similar in both primer and 5′RACE amplified sets (FIG. 2). In the 5′ RACE amplified dataset 55% of the VH genes were paired with J2 the next most frequently used J region was J3 while a similar proportion of J1 and J4 genes were used. The V-J pairing distribution was maintained after primer-set amplification in which J2 remained predominantly used (49%), followed by J3 (33%). Thus the frequency of the VH family usage was preserved before and after V region specific PCR.

The kappa light chain (VK) samples were similarly analysed. Here it was observed that the V-J pairing pattern was comparable in both 5′RACE and primer-set amplified products (FIG. 2). For example, the VK22-JK2 pairing frequency was observed at 55% and 50% in the 5′RACE and primer amplified samples respectively. The general frequency of the usage for each kappa V chain family and J genes did not show differences in the two samples.

The PCR products obtained after V region specific amplifications demonstrated a similar representation of VK families as the 5′RACE sample. IgVK7, IgVK13, IgVK18, IgVK20, IgVK21 were present at low frequency in the pre-PCR sample and were observed at a similar proportion post-amplification with the primer-set. A notable omission in the primer-set sample was the absence of IGKV13 which had a low representation in the 5′RACE dataset and a reduction in the frequency of the J4 region in the kappa light chain pairing. The larger proportion of J5-assigned sequences suggest that the J5 reverse primer, which shares homology with J4, may have preferentially amplified this region over the J4. Overall, both the frequency of the gene-usage and the V-J pairing suggest that PCR amplification using this primer-set did not skew the rat's immune repertoire, but enabled generation of V-J products as close as possible to the originally repertoire present in these rats.

### Example 3

Generation and Selection of an scFv Immune Phage Library from CD160-Vaccinated Rats

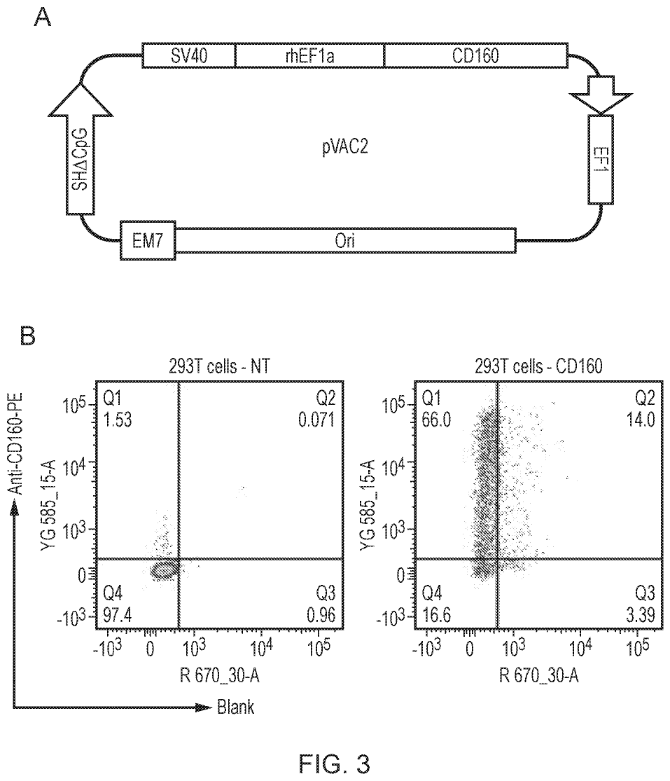

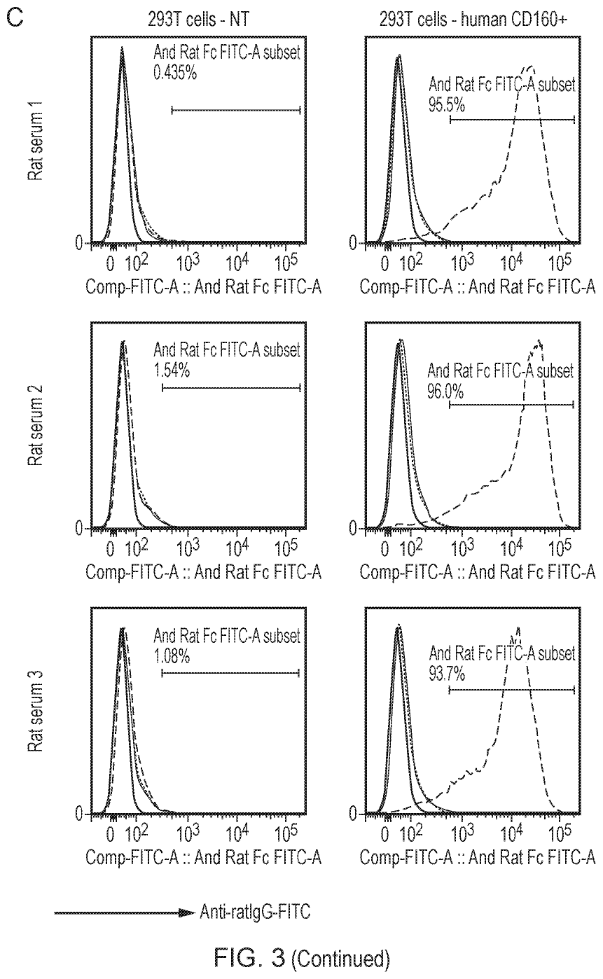

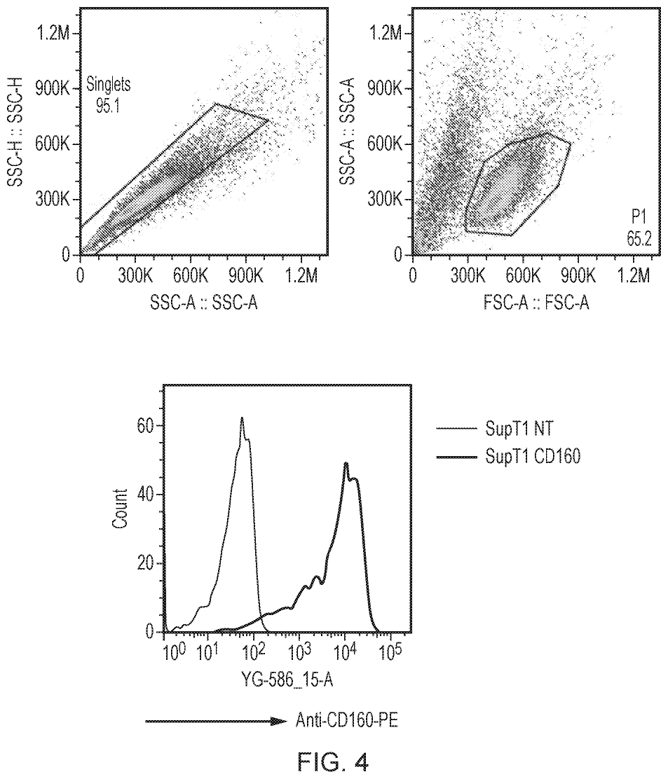

The human CD160 open reading frame was cloned into the pVAC2 expression vector (pVAC2.CD160). Expression was confirmed by transient transfection in 293T cells and staining with the commercial BY55 antibody (FIG. 3). CD160 was also cloned into the retroviral vector SFG and SupT1 cells expressing high levels of CD160 were generated (FIG. 4).

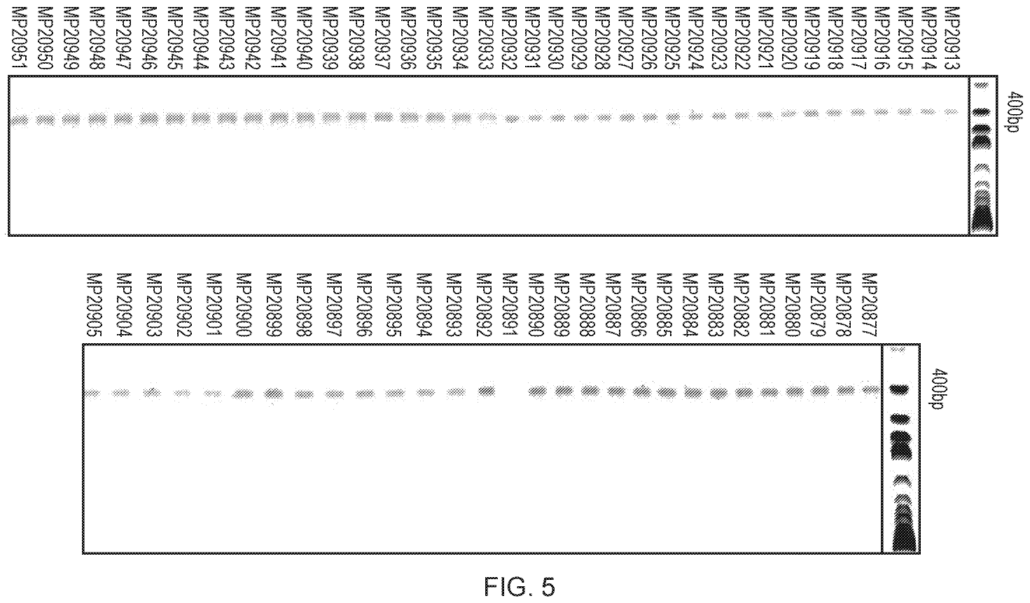

Three Wistar rats were genetically vaccinated using the plasmid pVAC2 encoding CD160. Twenty-one days post vaccination, serum-conversion was observed in all three rats (FIG. 3). Pooled cDNA from the three vaccinated rats was used as template with 68 individual PCR reactions, 29 for the VH and 39 for VK primers and reverse primers for the joining region where pooled together for each individual forward primer (FIG. 5). A notable feature was the presence of the VH family 3 which was sequenced at low frequency (0.4%) from 5′RACE amplicon in naïve rats. In the case of CD160 immunised animals this region was successfully amplified despite the fact that only one primer in the pool (M P20892) was designed to anneal to this region. After overlap extension PCR, the approximately 800 bp scFv insert was subsequently cloned in the phagemid vector and displayed on the phage surface for biopanning.

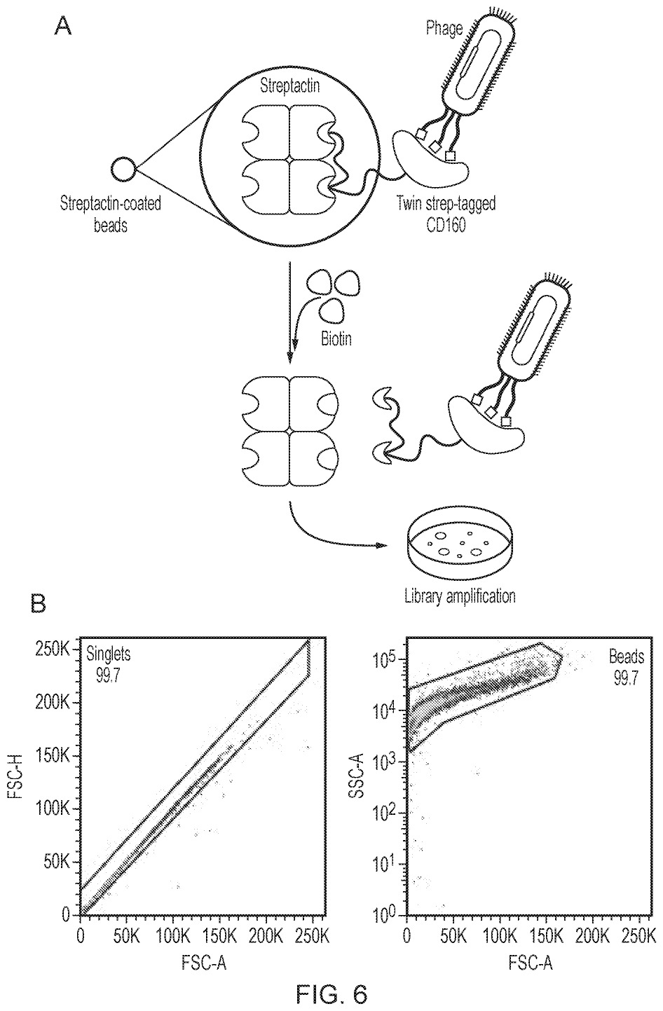

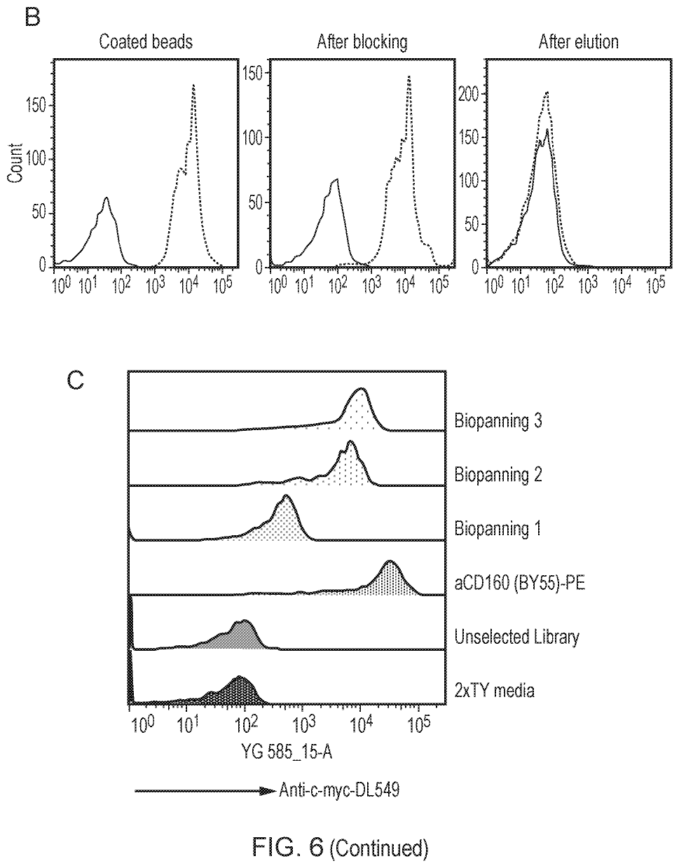

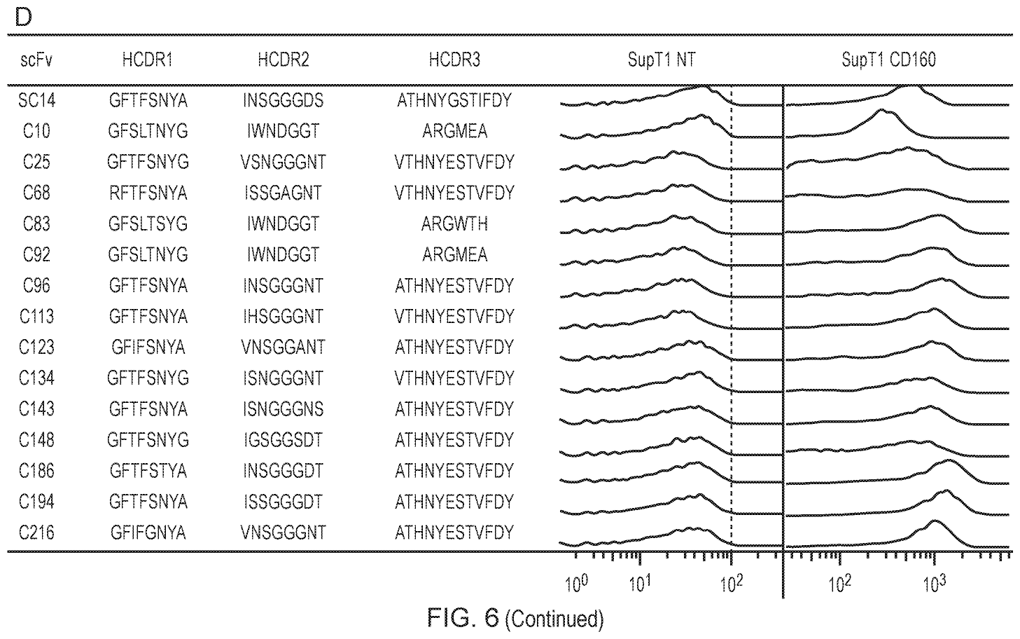

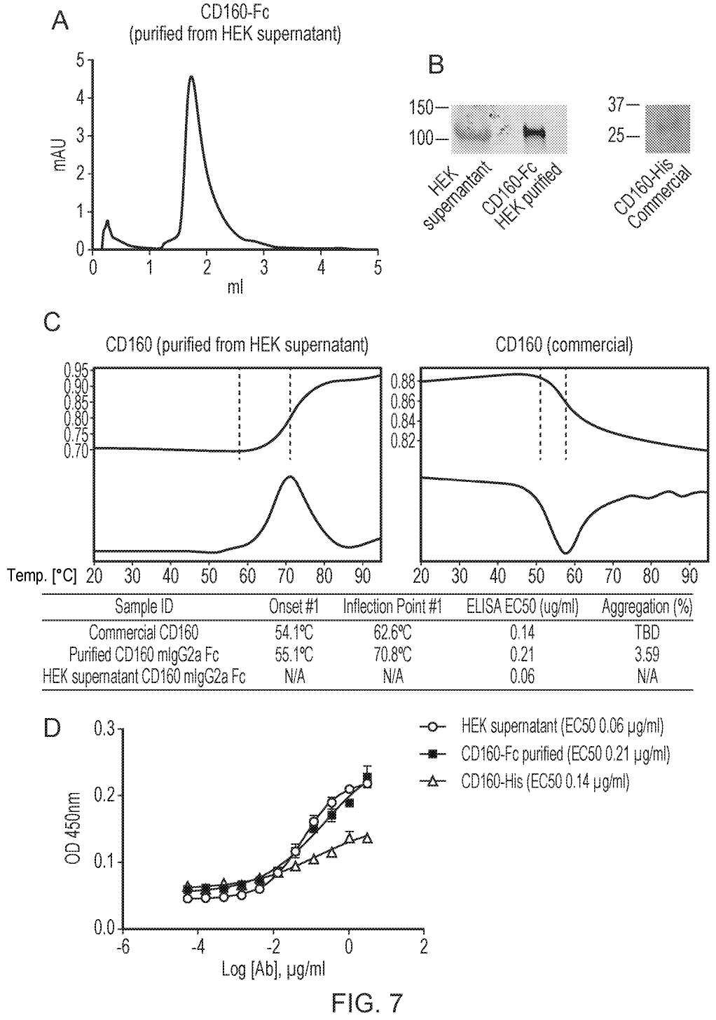

The human GPI-anchored protein CD160 was displayed on Strep-Tactin magnetic beads. This facilitated direct capture on to beads from the supernatant of transfected 293T cells and convenient elution with biotin (FIG. 6). To confirm quality of the CD160 protein from the transfected 293T was prepared to 95% purity, analysed by size exclusion chromatography (SEC) and differential scanning fluorimetry (DSF), and compared with different sources of CD160 protein (FIG. 7). Three sequential biopanning rounds of the phage-scFv library were conducted using the CD160-Strep-Tactin magnetic beads; enrichment was tested after each round of selection. To increase the likelihood of finding functional binders against the native conformation of the target antigen, individual clones were screened using flow cytometry. Successful enrichment was assessed using IPTG-induced bacterial supernatant to stain CD160 positive SupT1 cells (FIG. 6). A clear binding of the supernatant was observable in selection rounds 2 and 3, but a small enrichment is also evident in round one, highlighting the presence of specific binders after a single round of phage selection. Screening clones derived from individual bacterial colonies identified 15 unique scFvs that specifically bind CD160 positive cells (FIG. 6).

### Example 4

### Characterization of Identified Binders Revealed High Affinity Kinetic Clones

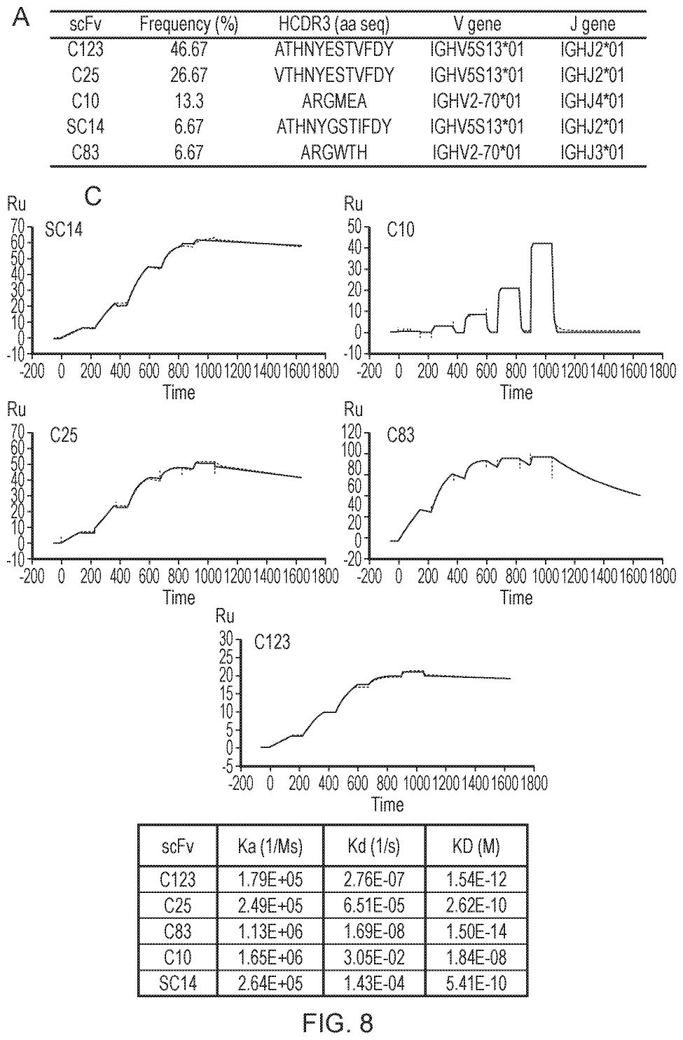

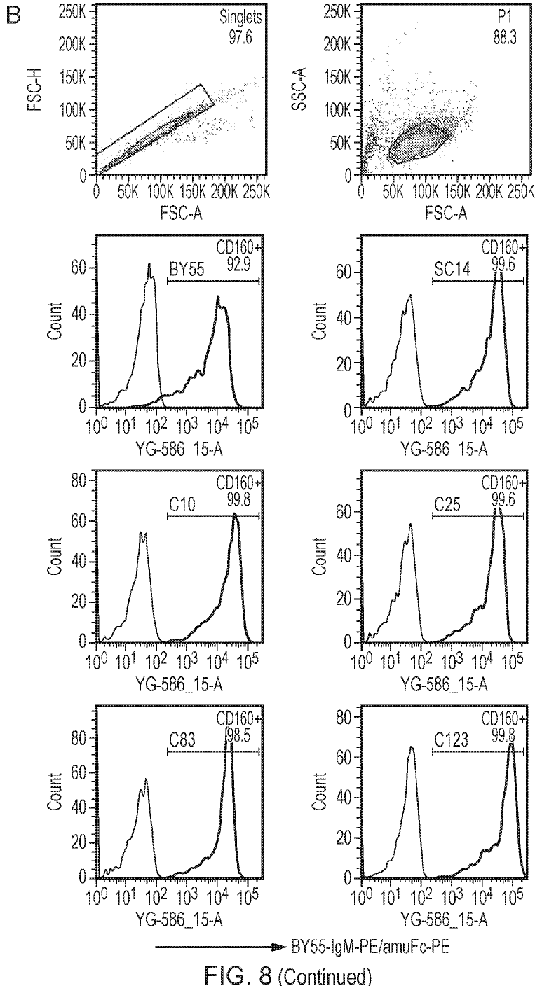

Alignment of the VH genes to the IMGT® database of rat germline variable genes revealed that the 15 unique scFv clones which contained 5 different HCDR3 originating from rearranged germline family V2 and V5. Two of these CDR3s were found to account for 46.67% and 26.67% of the total diversity (FIG. 8).

Five binders (clone 6 (SC14), clone 5 (010), clone 2 (C25), clone 3 (C83), clone 1 (C123)) carrying unique CDR3s were studied further as recombinant chimeric rat scFv/mouse IgG2aFc antibodies. Flow cytometric analysis determined that the recombinant antibodies selectively bound cell lines engineered to express CD160 (FIG. 8).

The kinetic profile of the scFvs was studied by SPR using purified CD160 as the analyte and a range of high affinity scFvs were found (FIG. 8). Binding, association and dissociation kinetics are presented in Table 7 below.

TABLE 7

| Clone | Ka (1/Ms) | Kd (1/s) | KD (M) |
|---|---|---|---|
| 1 | 1.79E+05 | 2.76E−07 | 1.54E−12 |
| 2 | 2.49E+05 | 6.51E−05 | 2.62E−10 |
| 3 | 1.13E+06 | 1.69E−08 | 1.50E−14 |
| 4 | 3.25E+05 | 3.90E−05 | 1.20E−10 |
| 5 | 1.65E+06 | 3.05E−02 | 1.84E−08 |
| 6 | 2.64E+05 | 1.43E−04 | 5.41E−10 |
| 7 | 1.06E+06 | 3.50E−04 | 3.30E−10 |
| 8 | 1.63E+06 | 8.04E−03 | 4.92E−09 |
| 9 | 4.08E+05 | 2.11E−03 | 5.18E−09 |
| 10 | 2.75E+05 | 2.45E−03 | 8.91E−09 |
| 11 | 1.37E+05 | 1.25E−03 | 9.15E−09 |
| 12 | 2.05E+05 | 1.75E−07 | 8.53E−13 |
| 16 | 2.79E+05 | 2.43E−03 | 8.72E−09 |
| 19 | 1.57E+05 | 8.33E−04 | 5.31E−09 |
| 20 | 3.81E+05 | 2.66E−03 | 6.98E−09 |

The range of kinetics observed in the clones suggest that the binders selected from the library will have utility in a wide range of applications.

### Example 5

CAR Engineered T-Cell were Functional and Able to Specifically Kill CD160 Positive Cells

The therapeutic potential of CD160 binders was next demonstrated converting the CD160 scFvs of clones 1 (C123), 2 (C25), 3 (C83), 5 (010) and 6 (SC14) into CAR format. A third generation CAR construct with CD28 and OX40 costimulatory endodomains and with a CD8STK as spacer moiety was selected as the format for CAR comparison.

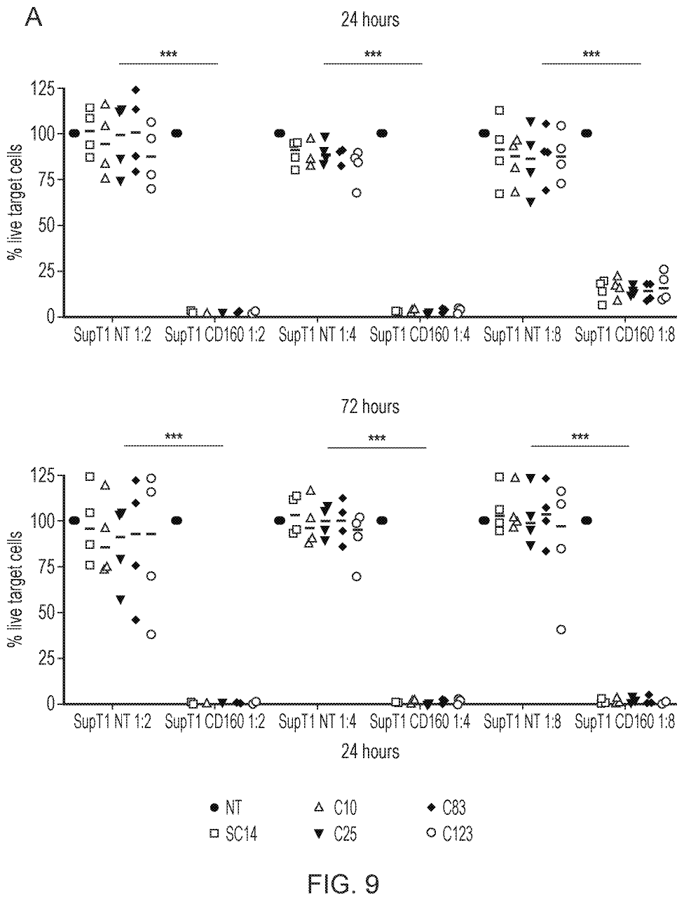

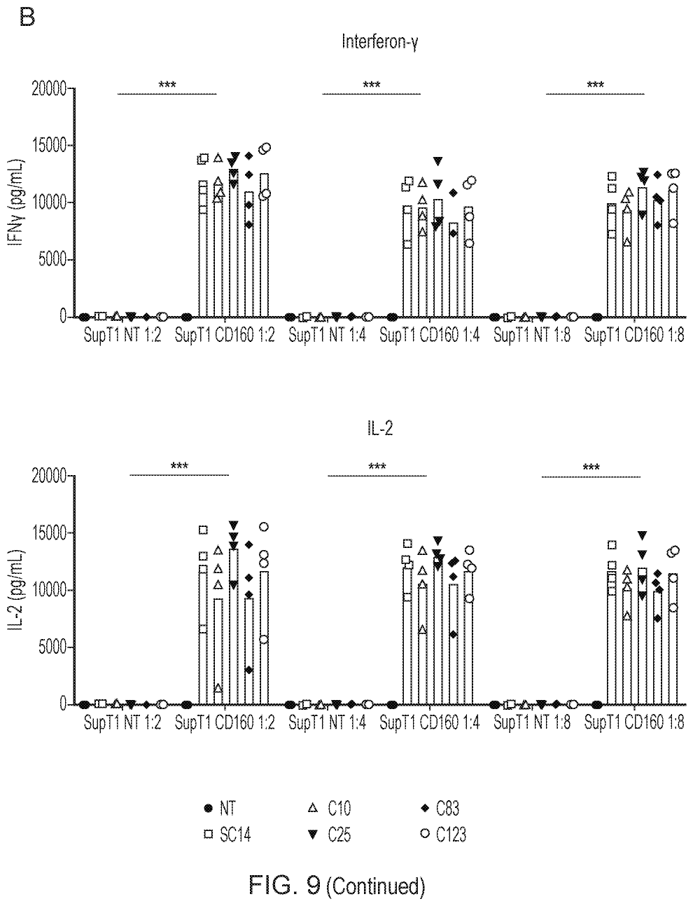

Normal donor, peripheral blood T-cells were transduced to express CAR constructs using gamma retroviral vectors. Transduced T-cells were then co-cultured at different effector to target ratios with either SupT1 cells (which do not express CD160) and SupT1 cells engineered to express CD160. One day post co-culture control of the SupT1 cells expressing CD160 was observed, with complete killing of the CD160 target cells by 72 h (FIG. 9).

Furthermore, the 5 different CARs showed no non-specific killing with complete recovery of the NT target cells at both time points.

The CD160 CARs were further evaluated for the secretion of pro-inflammatory cytokines interferon-gamma (IFN-γ) and Interleukin-2 (IL-2) at 72 h. All CARs tested showed high levels of cytokine production (FIG. 9). No background cytokine release was observed to SupT1 cells. No differences were observed at any of the effector to target ratios tested, suggesting equal potency of all CARs generated. An interesting feature of all CARs tested was the high levels of IL2 secretion observed. This particular feature could be beneficial in the improvement of CAR T cells persistence in vivo.

### Example 6—Functionality of Anti-CD160 BiTEs

The ability of the CD160 binders to function in a BiTE was assessed.

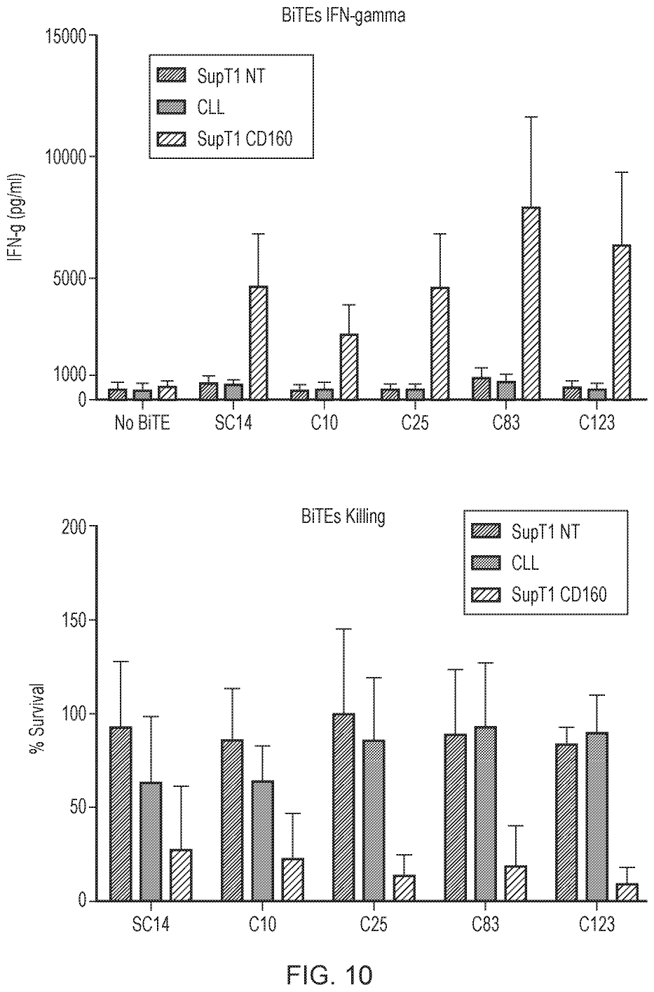

BiTEs were engineered using the ScFv and binders targeting the epsilon domain of CD3 using YTH as anti-CD3 ScFv (FIG. 10). The full BiTE structure consisted of a single molecule of the two ScFvs linked together with a CD8STK structure. The linker region was selected such that it would facilitate close contact between the target and effector cells upon binding of CD160 and CD3.

Co-culture experiments with target cells and effector T cells were performed for 48 hours and IFN-γ in the supernatant was assessed by ELISA. A target:effector ratio of 1:2 with control SupT1-NT (non-transfected negative control) cells, CD160 positive SupT1 cells and primary CLL cells were utilised as target cells.

All BiTE molecules demonstrated activation of effector cells with IFN-γ and specific cytotoxic activity induced by the engagement of CD160 positive cells but no activation with control target-negative cells (FIG. 10).

### Example 7—Binding of Anti-C160 Antibodies to CD160

### CD160 Staining of GPI and TM Isoforms

CD160 is know to exist in two different isoforms, the predominant format is CD160 GPI (095971), but it has also described to exist as a transmembrane protein (TM) (O95971-3).

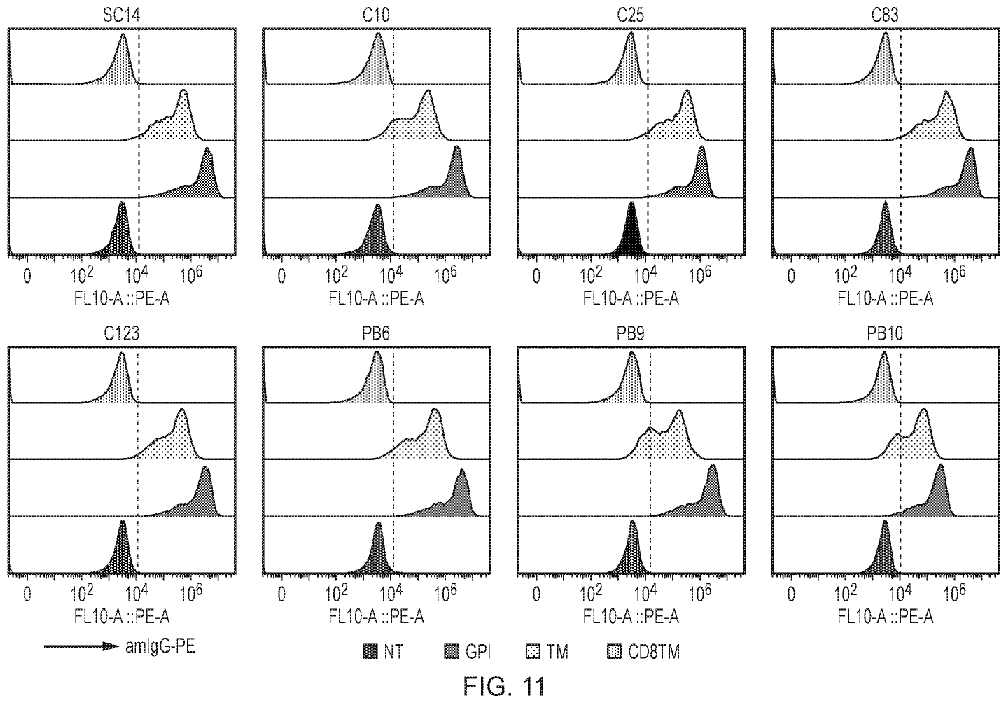

8 different anti-CD160 clones (clone 1 (C123), clone 2 (C25), clone 3 (C83), clone 5 (C10), clone 6 (SC14), clone 8 (PB9), clone 9 (PB6) and clone 11 (PB10)) were tested against the GPI and TM isoform of CD160 (FIG. 11). All the antibodies are able to specifically stain both the isoforms and not the negative controls (NT and CD8TM).

### Immunohistochemistry (IHC) Staining Optimization

Four anti-CD160 antibodies (1 mg/ml) were tested on FFPE sections of healthy tonsils.

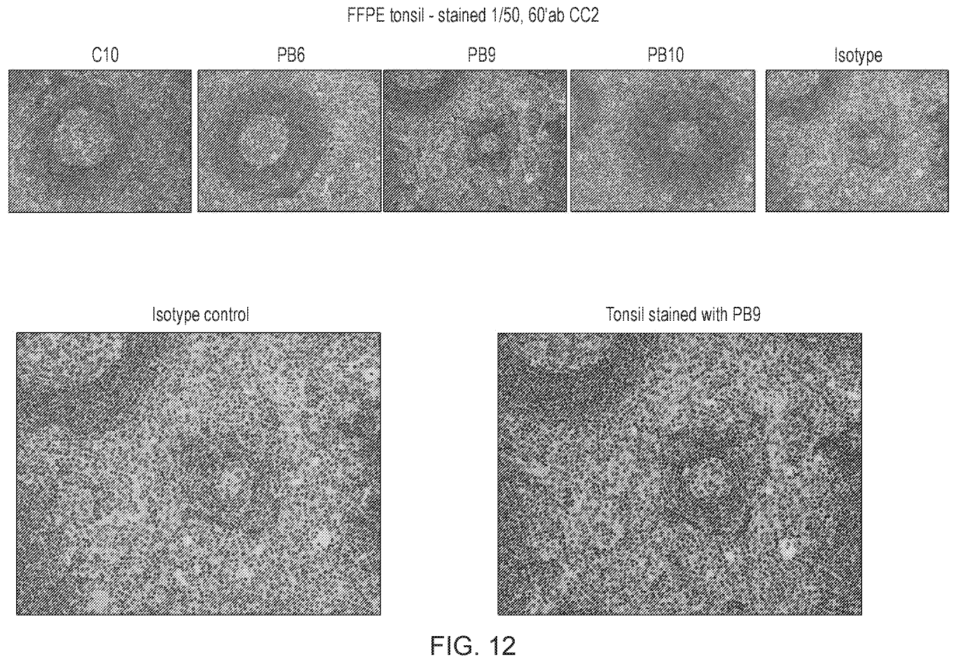

Four of the most different clones (clone 5 (010), clone 8 (PB9), clone 9 (PB6) and clone 11 (PB10)) were tested in IHC by staining healthy tonsils. Clone 8 (PB9) have shown positive staining when compared to the other clones and the isotype control (FIG. 12).

### NK and γδ T Cell Lines

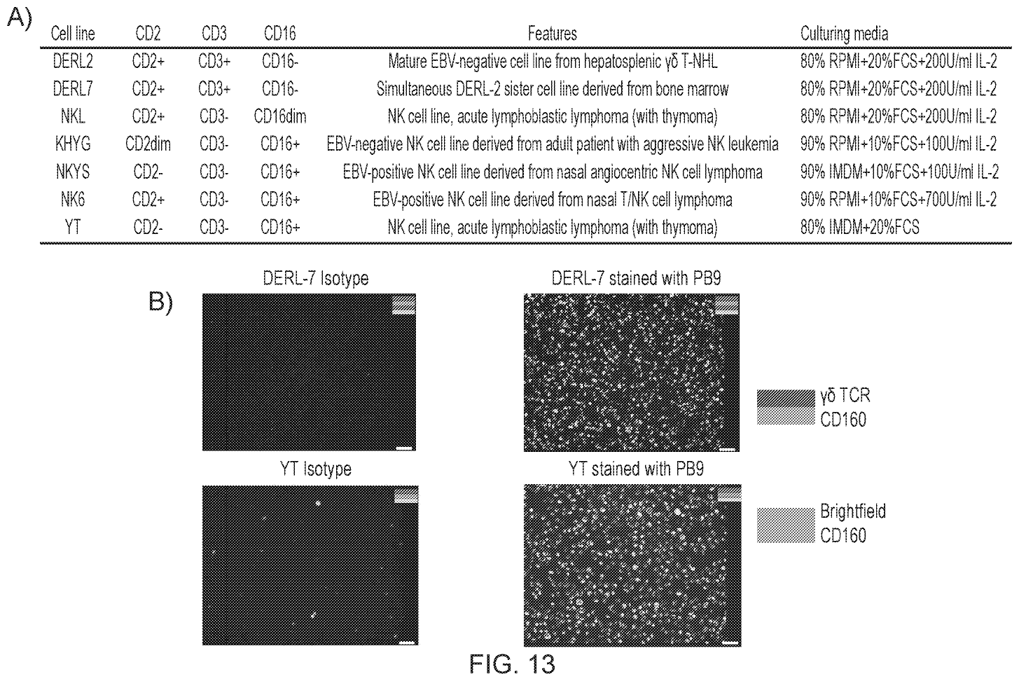

As CD160 is reported to be expressed on all healthy NK and gamma-delta T cells 7 different tumour cell lines were obtained to be used for the testing of CD160 expression (FIG. 13A).

Anti-CD160 clone 8 (PB9) have worked in IHC and it has been used to stain DERL7 and YT cell lines. Isotype control was used at matching concentration (1 mg/ml, 1/50) as negative control (FIG. 13B). Clone 8 (PB9) has shown to positively stain CD160 against the two cell lines when tested by chipcytometry.

Number of CD160 molecules on NK and γδ T cell lines

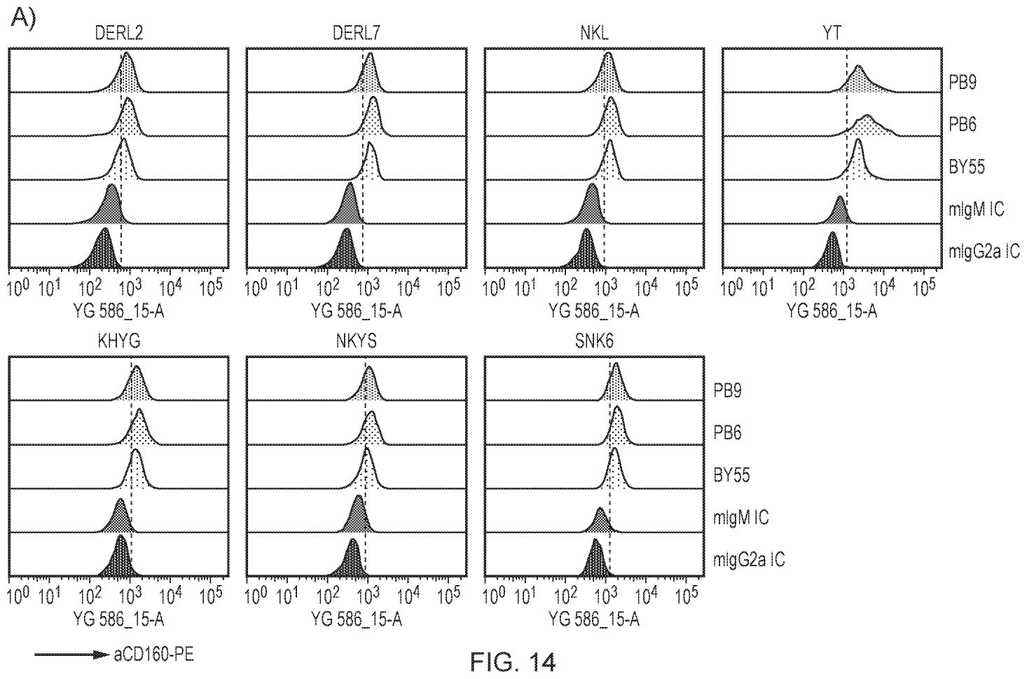

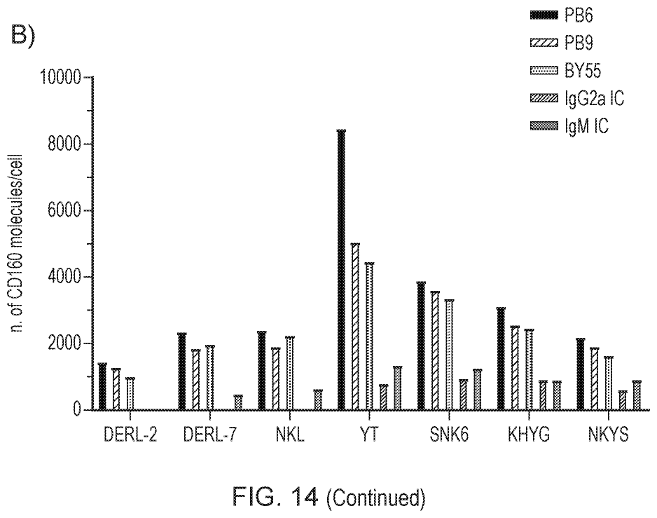

γδ and NK cell lines were stained with the 2 directly conjugated anti-CD160 antibodies (clone 9 (PB6) and clone 8 (PB9)), commercial BY55 was used for comparison (FIG. 14A). The number of molecules per cell was obtained using QuantiBrite-PE beads (FIG. 14B). Matching isotype control was used as negative control (IC).

All the NK and gamma-delta tumour cell lines express CD160 with a number of molecules between 1000 and 8000 per cell.

### Example 8—Anti-CD160 CAR-T Cell Generation and Testing

### Anti-CD160 CAR T Cell Generation

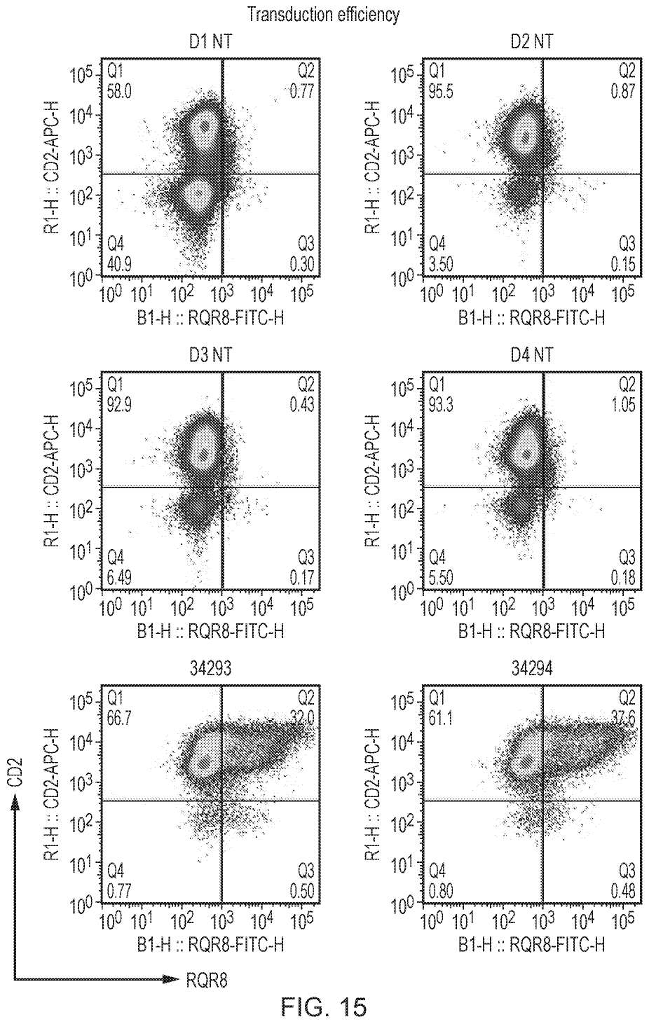

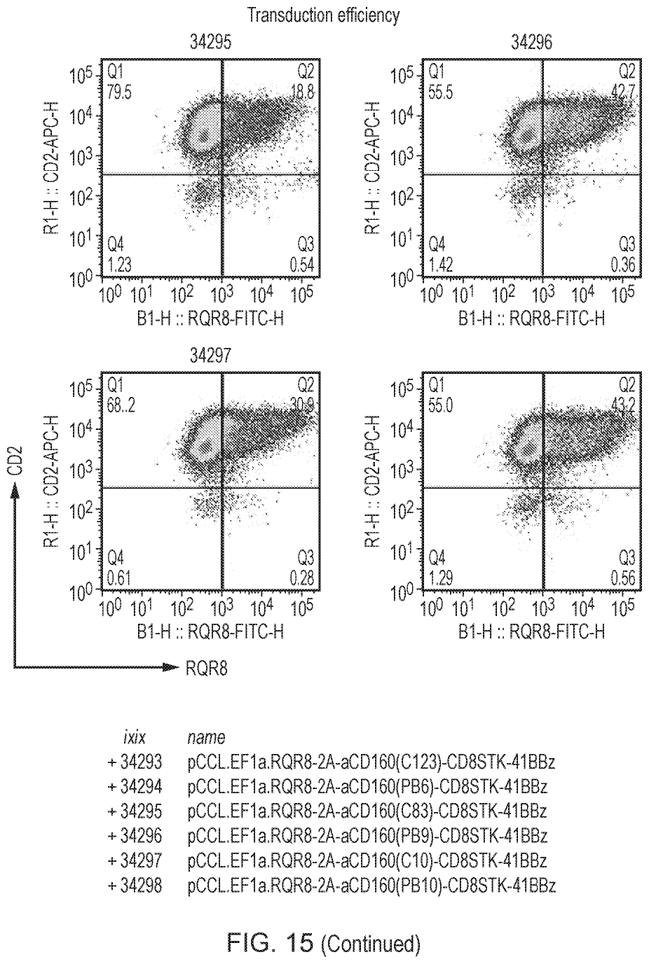

Second generation 4-1BBzeta CAR-T cell constructs harbouring 6 different anti-CD160 clones were generated (clone 1 (C123), clone 3 (C83), clone 5 (010), clone 8 (PB9), clone 9 (PB6) and clone 11 (PB10)). CARs were used to transduce 4 healthy donors demonstrating good expression by the staining of the marker gene (RQR8) (FIG. 15).

### Anti-CD160 CAR-T Cell Testing—Cytotoxicity

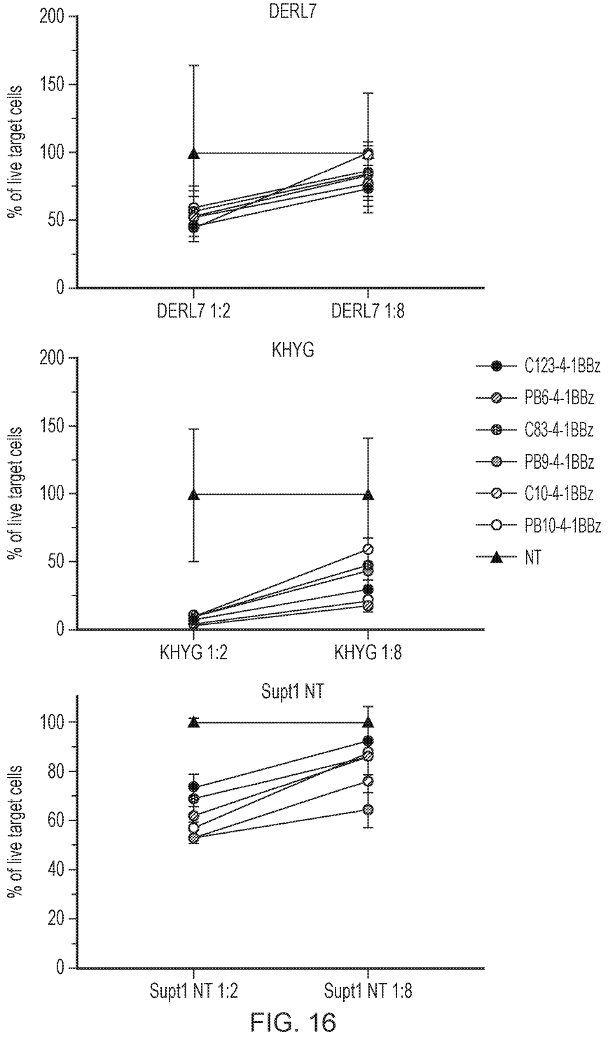

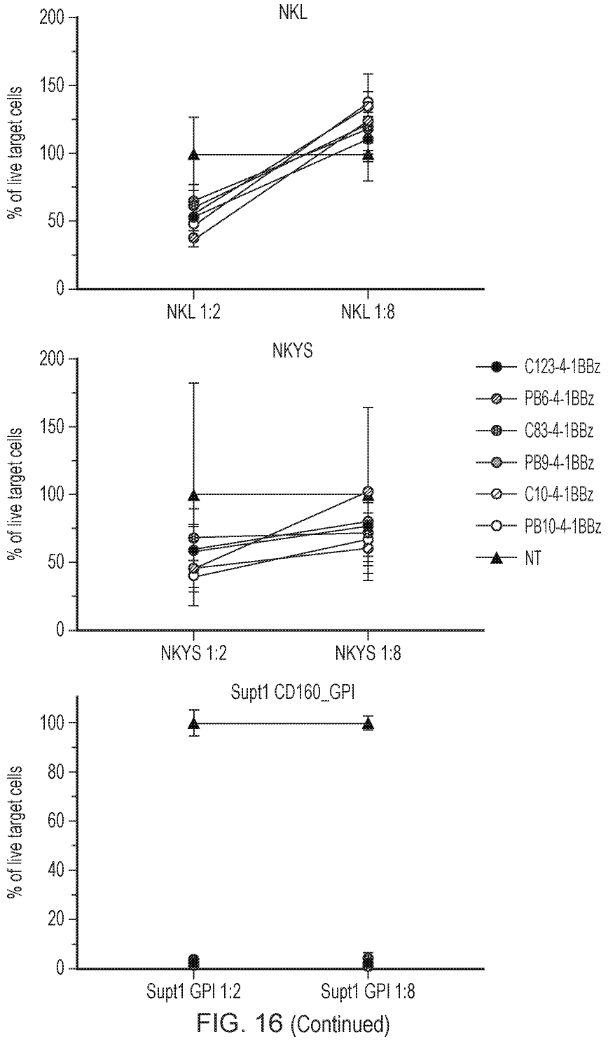

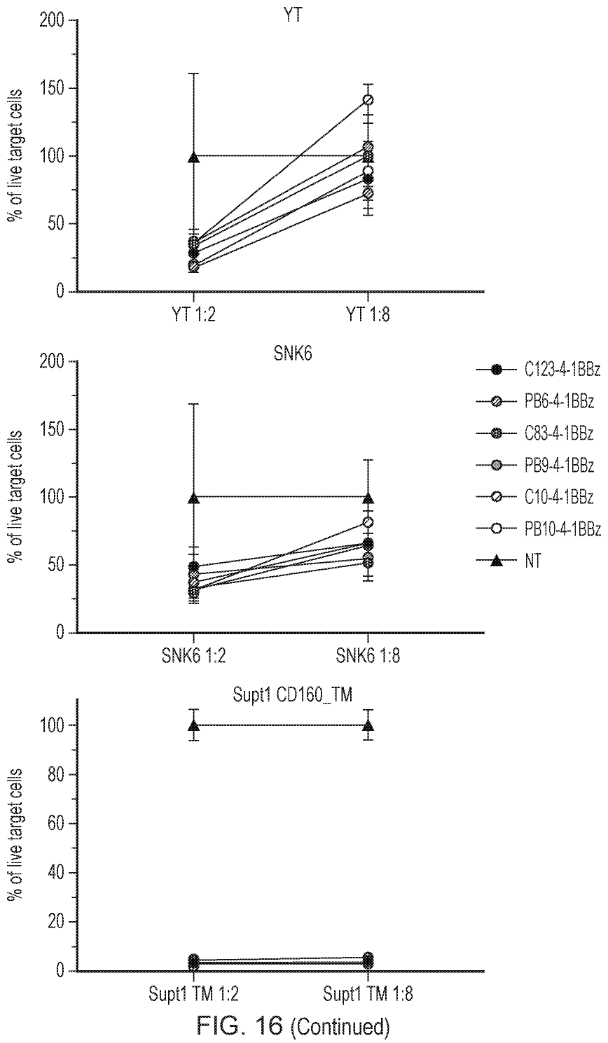

Cytotoxicity assay using the clone 1 (C123), clone 3 (C83), clone 5 (010), clone 8 (PB9), clone 9 (PB6) and clone 11 (PB10) CARs have shown different degrees of killing against NK and gamma-delta tumour cell lines (FIG. 16). The CARs were able to fully eradicate CD160-engineered cell lines (supt1 GPI and TM) with minimal background on CD160-negative cells (NT). Moreover, CAR-T cells have shown good cytotoxicity against YT and KHYG cell lines, demonstrating that CD160 could be a relevant target for NK malignancies. CD160 CAR-T cells have also shown to kill the gamma-delta cell line DERL7.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A CD160 binding domain, wherein the CD160 binding comprises:
(i) complementarity determining regions (CDRs) 1-3 comprising SEQ ID NOs 1-3; and CDRs 4-6 comprising SEQ ID NOs 4-6;
(ii) CDRs 1-3 comprising SEQ ID NOs 7-9; and CDRs 4-6 comprising SEQ ID NOs 4-6;
(iii) CDRs 1-3 comprising SEQ ID NOs 10-12; and CDRs 4-6 comprising SEQ ID NOs 13-15;
(iv) CDRs 1-3 comprising SEQ ID NOs 7, 16 and 17; and CDRs 4-6 comprising SEQ ID NOs 4-6;
(v) CDRs 1-3 comprising SEQ ID NOs 18, 11 and 19; and CDRs 4-6 comprising SEQ ID NOs 20-22;
(vi) CDRs 1-3 comprising SEQ ID NOs 23-25; and CDRs 4-6 comprising SEQ ID NOs 4-6;
(vii) CDRs 1-3 comprising SEQ ID NOs 26, 11 and 27; and CDRs 4-6 comprising SEQ ID NOs 28-30;
(viii) CDRs 1-3 comprising SEQ ID NOs 18, 11 and 31; and CDRs 4-6 comprising SEQ ID NOs 13, 32 and 33;
(xi) CDRs 1-3 comprising SEQ ID NOs 23, 34 and 35; and CDRs 4-6 comprising SEQ ID NOs 4-6;
(x) CDRs 1-3 comprising SEQ ID NOs 10, 11 and 36; and CDRs 4-6 comprising SEQ ID NOs 37, 14 and 38;
(xi) CDRs 1-3 comprising SEQ ID NOs 7, 39 and 40; and CDRs 4-6 comprising SEQ ID NOs 41-43;
(xii) CDRs 1-3 comprising SEQ ID NOs 7, 44 and 45; and CDRs 4-6 comprising SEQ ID NOs 4-6;
(xiii) CDRs 1-3 comprising SEQ ID NOs 46, 11 and 48; and CDRs 4-6 comprising SEQ ID NOs 49-51;
(xiv) CDRs 1-3 comprising SEQ ID NOs 7, 52 and 53; and CDRs 4-6 comprising SEQ ID NOs 4-6; or
(xv) CDRs 1-3 comprising SEQ ID NOs 23, 54 and 55; and CDRs 4-6 comprising SEQ ID NOs 4-6;
optionally wherein one or more of the CDRs comprises one, two or three amino acid mutations.

2. The CD160 binding domain according to claim 1, wherein the CD160 binding domain comprises:
(i) a VH region having the sequence shown as SEQ ID NO: 114 or a variant having at least 80% sequence identity thereto and a VL region having the sequence shown as SEQ ID NO: 115 or a variant of having at least 80% sequence identity thereto; or
(ii) a VH region having the sequence shown as SEQ ID NO: 116 or a variant having at least 80% sequence identity thereto and a VL region having the sequence shown as SEQ ID NO: 117 or a variant of having at least 80% sequence identity thereto; or
(iii) a VH region having the sequence shown as SEQ ID NO: 56 or a variant having at least 80% sequence identity thereto and a VL region having the sequence shown as SEQ ID NO: 57 or a variant of having at least 80% sequence identity thereto; or
(iv) a VH region having the sequence shown as SEQ ID NO: 118 or a variant having at least 80% sequence identity thereto and a VL region having the sequence shown as SEQ ID NO: 119 or a variant of having at least 80% sequence identity thereto; or
(v) a VH region having the sequence shown as SEQ ID NO: 120 or a variant having at least 80% sequence identity thereto and a VL region having the sequence shown as SEQ ID NO: 121 or a variant of having at least 80% sequence identity thereto; or
(vi) a VH region having the sequence shown as SEQ ID NO: 124 or a variant having at least 80% sequence identity thereto and a VL region having the sequence shown as SEQ ID NO: 125 or a variant of having at least 80% sequence identity thereto; or
(vii) a VH region having the sequence shown as SEQ ID NO: 126 or a variant having at least 80% sequence identity thereto; and a VL region having the sequence shown as SEQ ID NO: 127 or a variant of having at least 80% sequence identity thereto; or
(viii) a VH region having the sequence shown as SEQ ID NO: 130 or a variant having at least 80% sequence identity thereto and a VL region having the sequence shown as SEQ ID NO: 131 or a variant of having at least 80% sequence identity thereto.

3. The CD160 binding domain according to claim 1 or claim 2, wherein the CD160 binding domain comprises:
(i) the sequence shown as SEQ ID NO: 158 or a variant thereof at least 80% sequence identity thereto; or
(ii) the sequence shown as SEQ ID NO: 159 or a variant thereof at least 80% sequence identity thereto; or
(iii) the sequence shown as SEQ ID NO: 62 or a variant thereof having at least 80% sequence identity; or
(iv) the sequence shown as SEQ ID NO: 160 or a variant thereof at least 80% sequence identity thereto; or
(v) the sequence shown as SEQ ID NO: 161 or a variant thereof at least 80% sequence identity thereto; or
(vi) the sequence shown as SEQ ID NO: 163 or a variant thereof at least 80% sequence identity thereto; or
(vii) the sequence shown as SEQ ID NO: 164 or a variant thereof at least 80% sequence identity thereto; or
(viii) the sequence shown as SEQ ID NO: 166 or a variant thereof at least 80% sequence identity thereto.

4. An antibody or antigen-binding fragment thereof comprising the CD160 binding domain according to any preceding claim.

5. The antibody or fragment thereof according to claim 4, wherein the antibody or fragment thereof is a scFv, a monoclonal antibody or fragment thereof, or a humanized antibody or fragment thereof.

6. An antibody conjugate comprising the antibody or fragment thereof according to claim 4 or 5.

7. A chimeric antigen receptor (CAR) comprising a CD160 binding domain according to any preceding claim.

8. The CAR according to claim 7, which comprises a transmembrane domain which comprises the sequence selected from the group comprising SEQ ID NO: 65 or SEQ ID NO: 66, or a variant thereof having at least 80% sequence identity.

9. The CAR according to claim 7 or 8, wherein the CD160 binding domain and the transmembrane domain are connected by a spacer.

10. The CAR according to claim 9, wherein the spacer comprises one of the following: an IgG1 Fc domain; an IgG1 hinge; an IgG1 hinge-CD8 stalk; or a CD8 stalk.

11. The CAR according to claim 10, wherein the spacer comprises the sequence selected from the group comprising SEQ ID NO: 74, SEQ ID NO: 75, SEQ ID NO: 76, SEQ ID NO: 77 or SEQ ID NO: 78 or a variant thereof having at least 80% sequence identity.

12. The CAR according to any one of claims 7 to 11 which also comprises an intracellular T cell signalling domain.

13. The CAR according to claim 12 wherein the intracellular T cell signalling domain comprises one or more of the following endodomains: CD28 endodomain; 41BB endodomain; OX40 endodomain and CD3-Zeta endodomain.

14. The CAR according to claim 13 wherein the intracellular T cell signalling domain comprises all of the following endodomains: CD28 endodomain; 41BB endodomain; OX40 and CD3-Zeta endodomain.

15. The CAR according to any one of claims 7 to 14, which comprises the sequence selected from the group comprising SEQ ID NO: 79, SEQ ID NO: 80 or SEQ ID NO: 81, or a variant thereof which has at least 80% sequence identity thereto but retains the capacity to i) bind CD160 and ii) induce T cell signalling.

16. A T cell activator molecule which is a bi-specific molecule comprising:
(i) a first domain which comprises a CD160 binding domain according to any of one of claim 1-3; and
(ii) a second domain capable of activating a T cell.

17. The bi-specific molecule according to claim 16, wherein the second domain activates a T cell by binding CD3 on the T cell surface.

18. The bi-specific molecule according to claim 17, wherein the second domain comprises a CD3-specific antibody or part thereof.

19. The bi-specific molecule according to claim 18, wherein the second domain comprises the sequence selected from the group comprising SEQ ID NO: 90, SEQ ID NO: 97 or SEQ ID NO: 104 or a variant thereof which has at least 80% sequence identity and binds CD3.

20. The bi-specific molecule according to any of claims 16 to 19, wherein the first and second binding domains are connected by a spacer.

21. The bi-specific molecule according to claim 20, wherein the spacer comprises an IgG1 hinge or a CD8 stalk.

22. The bi-specific molecule according to any of claims 16 to 21, which comprises the sequence selected from the group comprising SEQ ID NO: 110, SEQ ID NO: 111 or SEQ ID NO: 112 or a variant thereof which has at least 80% sequence identity but retains the capacity to i) bind CD160 and ii) activate a T cell.

23. A polynucleotide comprising a nucleic acid sequence encoding a CD160 binding domain according to any of claims 1 to 3, an antibody or fragment thereof according to claim 4 or 5, a CAR according to any of claims 7 to 15 or a bi-specific molecule according to any of claims 16 to 22.

24. A vector which comprises a polynucleotide according to claim 23.

25. A cell which comprises a CAR according to any of claims 7 to 15.

26. The cell according to claim 25, wherein the cell is a T cell or a natural killer (NK) cell.

27. A cell comprising the polynucleotide according to claim 23 or a vector according to claim 24.

28. A method for making a cell according to any one of claims 25 to 27, which comprises the step of introducing a polynucleotide according to claim 23 or a vector according to claim 24 into said cell.

29. A pharmaceutical composition which comprises a CD160 binding domain according to any of claims 1 to 3, an antibody or fragment thereof according to claim 4 or 5, or an antibody conjugate according to claim 6, or a bi-specific molecule according to any of claims 16 to 22, or a vector according to claim 24, or a cell according to any one of claims 25 to 27, together with a pharmaceutically acceptable carrier, diluent or excipient.

30. A method for treating a disease which comprises the step of administering a CD160 binding domain according to any of claims 1 to 3, an antibody or fragment thereof according to claim 4 or 5, or an antibody conjugate according to claim 6, or a bi-specific molecule according to any of claims 16 to 22, or a vector according to claim 24 or a cell according to any one of claims 25 to 27 or a pharmaceutical composition according to claim 29 to a subject.

31. Use of a CD160 binding domain according to any of claims 1 to 3, an antibody or fragment thereof according to claim 4 or 5, or an antibody conjugate according to claim 6, or a bi-specific molecule according to any of claims 16 to 22, or a vector according to claim 24 or a cell according to any one of claims 25 to 27 or a pharmaceutical composition according to claim 29 in the manufacture of a medicament for treating a disease.

32. A CD160 binding domain according to any of claims 1 to 3, an antibody or fragment thereof according to claim 4 or 5, or an antibody conjugate according to claim 6, or a bi-specific molecule according to any of claims 16 to 22, or a vector according to claim 24 or a cell according to any one of claims 25 to 27 or a pharmaceutical composition according to claim 29 for use as a medicament in the treatment of a disease.

33. The method according to claim 30; the use according to claim 31; or the CD160 binding domain, the antibody or fragment, or the antibody conjugate, or the bi-specific molecule, or the vector, or the cell, or the pharmaceutical composition for use according to claim 32; wherein the disease is cancer.

34. The method, the use, the antibody or fragment, or the antibody conjugate, or the bi-specific molecule, or the vector, or the cell, or the pharmaceutical composition for use according to claim 33; wherein the cancer is selected from: NK lymphoma, TCRγδ lymphoma, chronic lymphocytic leukaemia, or hairy cell leukaemia.

35. The method according to claim 30; the use according to claim 31; or the CD160 binding domain, the antibody or fragment, or the antibody conjugate, or the bi-specific molecule, or the vector, or the cell, or the pharmaceutical composition for use according to claim 32; wherein the disease is associated with neoangiogenesis.
